(19) United States
(12) Patent Application Publication
(10) Pub. No.: US 2022/0040099 A1
(43) Pub. Date: Feb. 10, 2022

(54) INJECTABLE SOLUTION AT PH 7 COMPRISING AT LEAST A BASAL INSULIN WHICH PI IS COMPRISED BETWEEN 5.8 AND 8.5 AND A COPOLYAMINO ACID BEARING CARBOXYLATE CHARGES AND HYDROPHOBIC RADICALS

(71) Applicant: ADOCIA, Lyon (FR)

(72) Inventors: David DURACHER, Lyon (FR); Alexandre GEISSLER, Lyon (FR); Richard CHARVET, Rillieux la Pape (FR); Guilhem MORA, Lyon (FR); Romain NOEL, Villeurbanne (FR); You-Ping CHAN, Ternay (FR); Nicolas LAURENT, Miribel (FR)

(73) Assignee: ADOCIA, Lyon (FR)

(21) Appl. No.: 16/770,787

(22) PCT Filed: Dec. 4, 2018
(86) PCT No.: PCT/EP2018/083558

§ 371 Date: Oct. 18, 2021

Related U.S. Application Data
(60) Provisional application No. 62/606,138, filed on Dec. 7, 2017.

(30) Foreign Application Priority Data
Dec. 6, 2017 (FR) 1761736
Dec. 7, 2017 (FR) 17/61807
Jun. 29, 2018 (EP) 18181037.5
Jun. 29, 2018 (FR) 18/55934
Jun. 29, 2018 (FR) 18/56067

(51) Int. Cl. A61K 9/08; A61K 38/28; A61K 47/10; A61K 47/34; A61K 38/26

(52) U.S. Cl. CPC A61K 9/08; A61K 38/28; A61K 38/26; A61K 47/34; A61K 47/10

(57) ABSTRACT

A physically stable composition in the form of an injectable aqueous solution, wherein the pH is from 6.0 to 8.0, including at least: a) a basal insulin which isoelectric point (pI) is from 5.8 and 8.5 and b) a copolyamino acid according to formula I:

Q[Hy]ⱼ[PLG]ₖ Formula I

wherein: j≥1; k≥2.

The invention concerns insulin injection therapies for treating diabetes.

The invention relates to physically stable compositions in the form of an injectable aqueous solution, the pH of which is comprised from 6.0 to 8.0, comprising at least one basal insulin whose isoelectric point (pI) is comprised from 5.8 to 8.5, and a co-polyamino acid bearing carboxylate charges and hydrophobic radicals.

Insulin therapy, or diabetes treatment by insulin injection, has in recent years seen remarkable progress, specifically due to the development of new insulins, with a better blood sugar correction in patients in comparison with human insulin, and which make possible improved simulation of the physiological activity of the pancreas.

When type II diabetes is diagnosed in a patient, treatment is implemented gradually. First, the patient takes oral anti-diabetics (OAD) such as Metformin. When OADs alone are no longer sufficient to control the level of glucose in the blood, a change in treatment must be made and, depending on patient specificities, different treatment combinations can be implemented. For example, the patient may be treated with insulin glargine-type basal insulin or insulin detemir, in addition to OADs, then, depending on the evolution of the disease, with basal insulin and prandial insulin.

Furthermore, today, in order to make the transition from treatments by OADs, when the latter are no longer able to control the level of glucose in the blood, to a basal insulin/prandial insulin treatment, injection of GLP-1 RA analogues is recommended.

GLP-1 RA, for Glucagon-Like Peptide-1 receptor agonists, are insulinotropic peptides or incretins, and belong to the family of gastrointestinal hormones (or Gut Hormones) which stimulate the secretion of insulin when blood sugar is too high, for example, after a meal.

Gastrointestinal hormones (Gut hormones) are also called satiety hormones. Specifically, they comprise GLP-1 RA (Glucagon like peptide-1 receptor agonist) and GIP (Glucose-dependent insulinotropic peptide), oxyntomodulin (a derivative of proglucagon), the peptide YY, amylin, cholecystokinin, pancreatic polypeptide (PP), ghrelin and enterostatin, which are peptidic or proteic structures. They also stimulate the secretion of insulin in response to glucose and fatty acids and are, therefore, as such, potential candidates for the treatment of diabetes.

Among these, the GLP-1 RA are those that have provided, to date, the best results in the development of drugs. They have made it possible for patients affected by type II diabetes to lose weight, while maintaining better control of their blood sugar.

Thus, GLP-1 RA analogues or derivatives have been developed, in particular to improve their stability.

On the other hand, in order to meet his daily insulin needs, a diabetic patient currently has available, schematically, two types of insulins with complementary actions: prandial insulins (or so-called rapid-acting insulins) and basal insulins (or so-called slow-acting insulins)

Prandial insulins allow rapid management (metabolization and/or storing) of the glucose provided during meals and snacks. The patient must inject himself with a prandial insulin before each food intake, or about 2 to 3 injections per day. The most widely used prandial insulins are: recombinant human insulin, NovoLog® (NOVO NORDISK insulin aspart), Humalog® (ELI LILLY insulin aspart) and Apidra® (SANOFI insulin glulisine).

Basal insulins ensure the maintenance of the patient's glycemic homeostasis outside the periods of food intake. Essentially, they act to block the production of endogenous glucose (hepatic glucose). The daily dose of basal insulin generally corresponds to 40-50% of the total daily insulin needs. Depending on the basal insulin used, this dose is dispensed in 1 or 2 injections, regularly distributed over the course of the day. The most commonly used basal insulins are Levemir® (NOVO NORDISK insulin detemir) and Lantus® (SANOFI insulin glargine).

In the interest of being thorough, it should be noted that NPH (insulin NPH for Neutral Protamine Hagedorn; Humiline NPH®, Insulatard®) is the oldest basal insulin. This formulation is derived from precipitating human insulin (anionic at neutral pH) by a cationic protein, protamine. The microcrystals formed in this process are dispersed in an aqueous suspension and dissolve slowly after subcutaneous injection. This slow dissolution ensures extended insulin release. However, this release does not ensure a constant concentration of insulin over time. The release profile is bell-shaped and lasts only from 12 to 16 hours. Therefore, it is injected twice a day. This NPH basal insulin is much less effective than the modern basal insulins, Levemir® and Lantus®. NPH is an intermediate-acting basal insulin.

The principle of NPH evolved with the appearance of rapid insulin analogues which include products called “Premix” offering both rapid action and intermediate action. Novolog Mix® (NOVO NORDISK) and Humalog Mix® (ELI LILLY) are formulations comprising a rapid insulin analog, Novolog® and Humalog®, partially complexed with protamine. Thus, these formulations contain insulin analog micro-crystals, whose action is called intermediary, and a part of the insulin that remained soluble whose action is rapid. These formulations do offer the advantage of a rapid acting insulin, but they also have the disadvantage of NPH, namely, a duration of action limited to from 12 to 16 hours, and insulin released in a “bell” curve. However, these products allow the patient to inject an intermediate action basal insulin with a rapid-action prandial insulin. However, numerous patients are concerned about reducing the number of their injections.

Basal insulins currently on the market may be classified according to the technical solution that allows to obtain extended action and, presently, two approaches are used.

The first, that of insulin detemir, is the binding to albumin in vivo. It is an analog, soluble at pH 7, which comprises a fatty acid side chain (tetradecanoyl) attached to position B29 which, in vivo, allows this insulin to associate with albumin. Its extended action is principally due to this affinity for albumin after subcutaneous injection.

However, its pharmacokinetic profile does not allow it to last an entire day, so that it is most frequently used in two injections per day.

Another insulin soluble at pH 7 is insulin degludec, marketed under the name Tresiba®. It also comprises a fatty acid side chain attached to the insulin (hexadecantioyl-γ-L-Glu).

The second, that of insulin glargine, is the precipitation at physiological pH. Insulin glargine is an analog of human insulin obtained by elongation of the C-terminal part of the B chain of human insulin by two arginine residues, and by substitution of the asparagine residue A21 with a glycine residue (U.S. Pat. No. 5,656,722). The addition of two arginine residues was designed to adjust the pI (isoelectric point) of insulin glargine to the physiological pH, and thus to make this analog to human insulin insoluble in the physiological medium.

In addition, the substitution of A21 was designed in order to make insulin glargine stable at acidic pH and to thus be able to formulate it as an injectable solution at acidic pH. At the time of sub-cutaneous injection, the passage of insulin glargine from an acidic pH (pH 4-4.5) to a physiological pH (neutral pH) causes its precipitation under the skin. The slow redissolution of microparticle of insulin glargine ensures a slow and extended action.

The blood sugar lowering effect of insulin glargine is quasi-constant over a 24-hour period which allows most patients to only inject themselves once a day.

Insulin glargine is considered today as the most widely used basal insulin.

However, the necessarily acidic pH of basal insulin formulations, whose isoelectric point is comprised from 5.8 to 8.5, of insulin glargine type, may be a real problem, because this acidic pH of insulin glargine formulation sometimes causes pain at injection in patients and, especially, prevents any formulation with other proteins, in particular, with prandial insulins, because the latter are not stable at acidic pH. The impossibility to formulate a prandial insulin, at acidic pH, relates to the fact that prandial insulin undergoes, in these conditions, a secondary deamidation at position A21, which makes it impossible to meet the stability requirements applicable to injectable drugs.

At present, in applications WO 2013/021143 A1, WO 2013/104861 A1, WO 2014/124994 A1 and WO 2014/124993 A1, it was demonstrated that it was possible to solubilize these insulin glargine-type basal insulins, whose isoelectric point is comprised from 5.8 to 8.5, at neutral pH, while maintaining a difference in solubility between the in vitro medium (the container) and the in vivo medium (under the skin) regardless of the pH.

Application WO 2013/104861 A1 in particular describes compositions in the form of an injectable aqueous solution, whose pH is comprised from 6.0 to 8.0, comprising at least: a) one basal insulin whose isoelectric point (pI) is comprised from 5.8 to 8.5, and b) a copolyamino acid bearing carboxylate charges and hydrophobic radicals.

These compositions of the prior art have the major disadvantage of not being sufficiently stable to meet the specifications applicable to pharmaceutical formulations.

Therefore, there is a need to find a solution which allows to make a basal insulin soluble whose isoelectric point (pI) is comprised from 5.8 to 8.5, while preserving its basal profile after injection, but which also allows to satisfy the standard physical stability conditions for insulin-based pharmaceutical products.

Surprisingly, the applicant has found that the copolyamino acids that bear carboxylate charges and hydrophobic radicals according to the invention make it possible to obtain compositions as solutions which, not only meet the requirements described in WO 2013/104861 A1, but which also are able to provide improved physical stability to said compositions without having to increase the number of excipients used.

These performances, a priori never reached, are also maintained when the basal insulin whose isoelectric point is comprised from 5.8 to 8.5, is associated in the composition with a prandial insulin and/or a gastrointestinal hormone.

Thus, surprisingly, the affinity of copolyamino acids according to the invention for insulin glargine was increased in that it allows to obtain the solubilization and stabilization of insulin glargine solutions at a ratio [Hy]/[basal insulin] lower than that of the prior art; in addition, these results are obtained without altering, and are even improving, the propensity of insulin glargine to precipitate, as demonstrated in the experimental part.

This improvement in the affinity also makes it possible, in the context of chronic treatments, to limit the level of exposure to said excipients.

The copolyamino acids bearing carboxylate charges and hydrophobic radicals Hy according to the invention have an excellent resistance to hydrolysis. This can be specifically verified under accelerated conditions, for example, at basic pH (pH 12) by hydrolysis tests.

Moreover, forced oxidation tests, for example of the Fenton oxidation type, demonstrate that the co-polyamino acids bearing carboxylate charges and hydrophobic radicals Hy exhibit a good resistance to oxidation.

Thus the invention relates to physically stable compositions in the form of an injectable aqueous solution, which pH is comprised from 6.0 to 8.0, comprising at least:

a) a basal insulin which isoelectric point (pI) is comprised from 5.8 and 8.5 and

b) a copolyamino acid according to formula I′

Q[Hy]_{j]}[PLG]ₖ[Hy]_{hy}[Hy]_{hy′} Formula I′

### Wherein:

j≥1; k≥2
hy≥0 and hy′≥0
said copolyamino acid according to formula I′ bearing carboxylate charges and consisting of at least two chains of PLG glutamic or aspartic units bound together by a linear or branched radical or spacer Q[-*]ᵢ with i=j+k) at least trivalent consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl groups said radical Q[-*]ᵢ bearing at least a monovalent hydrophobic radical -Hy;
said radical or spacer Q[-*]ᵢ being bound to at least two glutamic or aspartic units PLG chains by an amide function and,
said radical or spacer Q[-*]ᵢ being bound to at least a hydrophobic radical
Hy according to formula X hereafter defined by an amide function.
said amide functions binding said radical or spacer Q[-*]ᵢ to the at least two chains of glutamic or aspartic units comes from the reaction between an amine function and an acid function respectively carried by either the precursor Q′ of the radical or spacer Q[-*]ᵢ or a glutamic or aspartic unit.
the amide function binding said radical or spacer Q[-*]ᵢ to, at least a hydrophobic radical -Hy according to formula X from the reaction between an amine function and an acid function carried either by the precursor Q′ of the radical or spacer Q[-*]ᵢ or by the precursor Hy′ of the hydrophobic radical -Hy; and
when hy and hy′≠0 then at least a hydrophobic radical -Hy is bound to either a terminal «amino acid» unit, or to a carboxyl function carried by one of the glutamic or aspartic units of the PLG chains.

In one embodiment, hy and hy′ are equal to 0 and thus the invention relates to physically stable compositions in the form of an injectable aqueous solution, which pH is comprised from 6.0 to 8.0, comprising at least:

a) a basal insulin which isoelectric point (pI) is comprised from 5.8 and 8.5 and

b) a copolyamino acid according to formula I

Q[HY]ᵢ[PLG]ₖ Formula I

### Wherein:

j≥1; k≥2
said copolyamino acid according to formula I bearing carboxylate charges and consisting of at least two chains of PLG glutamic or aspartic units bound together by a linear or branched radical or spacer Q[-*], with i=j+k) at least trivalent consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl groups said radical Q[-*]ᵢ bearing at least a monovalent hydrophobic radical -Hy according to formula X;
said radical or spacer Q[-*]ᵢ being bound to at least two chains of PLG glutamic or aspartic units by an amide function and,
said radical or spacer Q[-*]ᵢ being bound to at least a hydrophobic radical -Hy according to formula X by an amide function.
said amide functions binding said radical or spacer Q[-*]ᵢ to at least two chains of glutamic or aspartic units come from the reaction between an amine function and an acid function respectively carried by either the precursor Q′ of the radical or spacer Q[-*]ᵢ or by a glutamic or aspartic unit.
the amide function binding said radical or spacer Q[-*]ᵢ to, at least a hydrophobic radical -Hy according to formula X comes from the reaction between an amine function and an acid function carried by either the precursor Q′ of the radical or spacer Q[-*], or by the precursor Hy′ of the hydrophobic radical -Hy.

The pH of the compositions according to the invention is comprised from 6.0 to 8.0, preferably comprised from 7 to 7.8, preferably comprised from 6.6 and 7.8 or even more preferably between 6.8 to 7.6.

Said copolyamino acid bearing carboxylate charges and at least a hydrophobic radical -Hy is soluble in aqueous solution at a pH comprised from 6.0 to 8.0, at a temperature of 25° C. and at a concentration at least equal to 60 mg/ml.

The term «physically stable composition» refers to compositions that meet the visual inspection criteria described in European, American and international pharmacopoeia, that is, compositions that are clear and that do not contain visible particles, but are also colorless.

By “injectable aqueous solution” is meant solutions for which the solvent is water and which meet the pharmacopoeia conditions of Europe and the US.

The term “copolyamino acid consisting of glutamic or aspartic units” refers to non-cyclic linear chains of glutamic acid or aspartic acid units bound together by peptide bonds, said chains having a C-terminal part, corresponding to the carboxylic acid of one extremity, and an N-terminal part, corresponding to the amine of the other extremity of the chain.

The term “soluble” used herein means suitable to prepare a clear, particle-free solution at a concentration below 60 mg/ml in distilled water at 25° C.

The term “alkyl radical” denotes a linear or branched carbon chain, which does not comprise a heteroatom.

Said copolyamino acid is a statistical copolyamino acid in the chain of glutamic and/or aspartic units.

In the formulas, the * indicate the binding sites of the different elements represented.

In one embodiment, the composition according to the invention is characterized in that Hy comprises from 30 to 70 carbon atoms.

In one embodiment, the radical or spacer Q[-*]ᵢ (i≥3) is represented by a radical according to formula II:

Q[-*]ᵢ=([Q′]_{q})[-*]ᵢ Formula II

Wherein q 5

The radicals Q′ being identical or different and chosen in the group consisting of radicals according to the following formulas III to VI, to form Q[-*]ᵢ (i≥3)

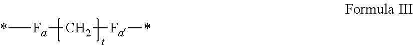

Wherein 1≤t≤8

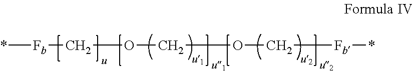

Wherein:

At least one of u₁″ or u₂″ is different from 0.

If u₁″≠0 then u₁′≠0 and if u₂″≠0 then u₂′≠0, u₁′ and u₂′ are identical or different and,

2≤u≤4,

0≤u₁′≤4,

0≤u₁″≤4,

0≤u₂′≤4

0≤u₂″≤4 and,

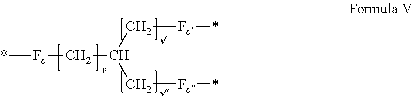

Wherein:

v, v′ and v″ identical or different,

v+v′+v″≤15

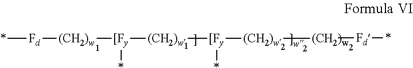

Wherein:

w₁′ is different from 0,

0≤w₂″≤1,

w₁≤6 and w₁′≤6 and/or w₂≤6 and w₂′≤6

with Fx=Fa, Fb, Fc, Fd, Fa′, Fb′, Fc′, Fc″ and Fd′ identical or different representing functions —NH— or —CO— and Fy representing a trivalent nitrogen atom —N═,

two radicals Q′ being bound between them by a covalent bond between a carboxyl function, Fx=—CO—, and an amine function Fx=—NH— or Fy=—N═, thus forming an amide bond,

In one embodiment, if Fa and Fa′ are —NH—, then

In one embodiment, if Fa and Fa′ are —CO—, then

In one embodiment, if Fa and Fa′ are —CO— and —NH—, then

In one embodiment, if Fb and Fb′ are —NH—, then u and and/or u₂′≥2.

In one embodiment, if Fc, Fc′ and Fc″ are —NH— then at least two of v, v′ and v″ are different from 0.

In one embodiment, if Fc, Fc′ and Fc″ are 2 —NH— and 1 —CO— then at least one of the subscripts of —(CH₂)— bearing a nitrogen is different from 0.

In one embodiment, if Fc, Fc′ and Fc″ are 1 —NH— and 2 —CO— then there is no conditions.

In one embodiment, if Fc, Fc′ and Fc″ are —CO— then at least one of the v, v′ and v″ is different from 0.

In one embodiment, if Fd and Fd′ are —NH—, w1 and w1′≥2 and/or w2 and w′2≥2.

In one embodiment, if Fd and Fd′ are —CO—, w1 and w1′≥1 and/or w2 and w2′≥1.

In one embodiment, if Fd and Fd′ are —CO— and —NH—, w1 and w1′≥1 and/or w2 and w2′≥1.

Hy and PLG being bound to Q[-*]ᵢ via a Fx or Fy function by a covalent bond to form an amide bond with a —NH— or —CO— function of the PLG or of the Hy.

In one embodiment, 1≤q≤4.

In one embodiment, v+v′+v″≤15.

In one embodiment, at least one of Q′ is a radical according to formula

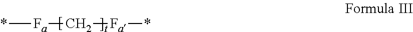

which precursor is a diamine.

In one embodiment, the precursor of the radical according to formula III is a diamine chosen in the group consisting of ethylene diamine, butylenediamine, hexylenediamine, 1,3-diaminopropane and 1,5-diaminopentane.

In one embodiment, t=2 and the precursor of the radical according to formula III is ethylene diamine.

In one embodiment, t=4 and the precursor of the radical according to formula III is butylenediamine.

In one embodiment, t=6 and the precursor of the radical according to formula III is hexylenediamine.

In one embodiment, t=3 and the precursor of the radical according to formula III is 1,3-diaminopropane.

In one embodiment, t=5 and the precursor of the radical according to formula III is 1,5-diaminopentane.

In one embodiment, the precursor of the radical according to formula III is an amino acid.

In one embodiment, the precursor of the radical according to formula III is an amino acid chosen in the group consisting of aminobutanoic acid, =aminohexanoic acid and =beta-alanine.

In one embodiment, t=2 and and the precursor of the radical according to formula III is beta-alanine.

In one embodiment, t=6 and and the precursor of the radical according to formula III is aminohexanoic acid.

In one embodiment, t=4 and the precursor of the radical according to formula III is aminobutanoic acid.

In one embodiment, the precursor of the radical according to formula III is a diacid.

In one embodiment, the precursor of the radical according to formula III is a diacid chosen in the group consisting of succinic acid, glutaric acid and adipic acid.

In one embodiment, t=2 and and the precursor of the radical according to formula III is succinic acid.

In one embodiment, t=3 and the precursor of the radical according to formula III is glutaric acid.

In one embodiment, t=4 and the precursor of the radical according to formula III is adipic acid.

In one embodiment, at least one of Q′ is a radical according to formula IV,

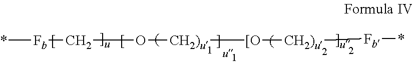

which precursor is a diamine.

In one embodiment, the precursor of the radical according to formula IV is a diamine chosen in the group consisting of diethyleneglycoldiamine, triethyleneglycol diamine, 1-amino-4,9-dioxa-12-dodecanamine and 1-amino-4,7.10-trioxa-13-tridecanamine.

In one embodiment, u=u′₁=2, u″₁=1, u″₂=0 and the precursor of the radical according to formula IV is diethyleneglycol diamine.

In one embodiment, u=u′₁=u′₂=2, u″₁=u″₂=1 and the precursor of the radical according to formula IV is triethyleneglycol diamine.

In one embodiment, u=u′₂=3, u′₁=4, u″₁=u″₂=1 and the precursor of the radical according to formula IV is 4,9-dioxa-1.12-dodecanediamine.

In one embodiment, u=u′₂=3, u′₁=u″₁=2, u″₂=1 and the precursor of the radical according to formula IV is 4,7,10-trioxa-1,13-tridecanediamine.

In one embodiment, at least one of Q′ is a radical according to formula V,

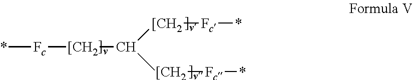

which precursor is chosen in the group consisting of amino acids.

In one embodiment, the precursor of the radical according to formula V is an amino acid chosen in the group consisting of lysine, ornithine and acid 2,3-diaminopropionic.

In one embodiment, at least one of Q′ is a radical according to formula V,

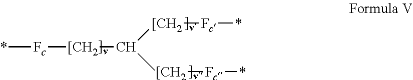

which precursor is chosen in the group consisting of triacids.

In one embodiment, the precursor of the radical according to formula V is a triacid chosen in the group consisting of tricarballylic acid.

In one embodiment, v=0, v′=v″=1 and the precursor of the radical according to formula V is tricarballylic acid.

In one embodiment, at least one of Q′ is a radical according to formula V,

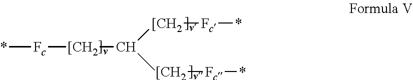

which precursor is chosen in the group consisting of triamines.

In one embodiment, the precursor of the radical according to formula V is a triamine chosen in the group consisting of (2-(aminomethyl)propane-1,3-diamine).

In one embodiment, v=v′=v″=1 and the precursor of the radical according to formula V is (2-(aminomethyl)propane-1,3-diamine).

In one embodiment, at least one of Q′ is a radical according to formula VI,

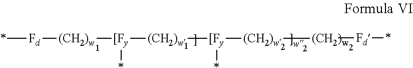

which precursor is a triamine.

In one embodiment, w″₂=0 and the precursor of the radical according to formula VI is a triamine chosen in the group consisting of spermidine, norspermidine, and diethylenetriamine and bis(hexamethylene)triamine.

In one embodiment, w″₂=0 and the precursor of the radical according to formula VI is spermidine.

In one embodiment, w″₂=0 and the precursor of the radical according to formula VI is norspermidine.

In one embodiment, w″₂=0 and the precursor of the radical according to formula VI is diethylenetriamine.

un mode de realisation, w″₂=0 and the precursor of the radical according to formula VI is bis(hexamethylene)triamine.

In one embodiment, w″₂=1 and the precursor of the radical according to formula VI is a tetramine.

In one embodiment, w″₂=1 and the precursor of the radical according to formula VI is a tetramine chosen in the group consisting of spermine and triethylenetetramine.

In one embodiment, w″₂=1 and the precursor of the radical according to formula VI is spermine.

In one embodiment, w″₂=1 and the precursor of the radical according to formula VI is triethylenetetramine. In one embodiment, PLG are bound to Fx with Fx=—NH— or to Fy by at least a carboxyl function of the PLG.

In one embodiment, PLG are bound to Fx with Fx=—NH— or to Fy by at least a carboxyl function which is not in the C terminal position of the PLG.

In one embodiment, PLG are bound to Fx with Fx=—NH— or to Fy by the carbonyl function in the C terminal position of the PLG.

In one embodiment, PLG are bound to Fx with Fx=—NH— by the carbonyl function in the C terminal position of the PLG.

In one embodiment, PLG are bound to Fx with Fx=Fy by the carbonyl function in the C terminal position of the PLG.

In one embodiment, Hy are bound to Fx with Fx=—NH— or to Fy by a carboxyl function of Hy carried by GpR, GpA, GpG, GpH, GpL or GpC.

In one embodiment, Hy are bound to Fy by a carboxyl function of Hy carried by GpR, GpA, GpG, GpH, GpL or GpC.

In one embodiment, Hy are bound to Fx with Fx=—NH— by a carboxyl function of Hy carried by GpR, GpA, GpG, GpH, GpL or GpC.

In one embodiment, PLG are bound to Fx, with Fx=—CO— by the nitrogen atom in the N terminal position of the PLG.

In one embodiment, Hy are bound to Fx with Fx=—CO— by a nitrogen atom of Hy carried by GpR, GpA, GpG, GpL or GpH.

In one embodiment the q Q′ are chosen in the group consisting of radicals according to formula VI, III and IV and Q comprises a radical according to formula VI with q≥1 and Q[-*]i is a radical in which i=3

and said copolyamino acid is a copolyamino acid according to general formula I:

Q[HY]ⱼ[PLG]ₖ Formula I
With j=1 and k=2
Hy according to formula X is bound to Q′ via a covalent bond to Fa, Fa′, Fb, Fb′, Fd, Fd′ or Fy thus forming an amide bond
the 2 PLG chains being bound to Q′ via a covalent bond to Fa, Fa′, Fb, Fb′, Fd, Fd′ or Fy, thus forming an amide bond.

In one embodiment q=1.

In one embodiment, Hy is bound to Q′ via a covalent bond between Fy and a carboxyl function of Hy carried by GpR, GpA, GpG, GpH, GpC or GpL to form an amide bond.

In one embodiment, PLG are bound to Q′ via a covalent bond between Fd, Fd′ (Fd and Fd′=—NH—) and the carbonyl function in the C terminal position of the PLG chain, thus forming an amide bond.

In one embodiment Q′ is a radical according to formula VI.

In one embodiment Q′ is a radical according to formula VI wherein:
w₂=w″₂=0 and 3≤w₁≤4 and 3≤w₁′≤4.

In one embodiment, Q′ is a radical according to formula VI wherein w₂=w″₂=0 and w₁=3 and w₁′=4.

In one embodiment, Q′ is a radical according to formula VI wherein w₂=w″₂=0 and w₁=w₁′=3.

In one embodiment Fd=Fd′=—NH— and are bound each independently by a covalent bond via a terminal carbonyl function of the PLG forming an amide bond and Fy is bound by a covalent bond at a carbonyl function of the hydrophobe Hy forming an amide bond.

In one embodiment q Q′ are chosen in the group consisting of the radicals according to formula III, IV and V and Q comprises at least a radical according to formula V with q 1 and Q[-*]i is a radical in which i=3

and said copolyamino acid is a copolyamino acid according to general formula I:

Q[HY]ⱼ[PLG]ₖ Formula I
With j=1 and k=2
Hy being according to formula X, bound to Q′ via a covalent bond to Fc, Fc′, Fc″, Fb, Fb′, Fa or Fa′ thus forming an amide bond
the 2 PLG chains being bound to Q′ via a covalent bond with Fc, Fc′, Fc″, Fb, Fb′, Fa or Fa′, thus forming an amide bond.

In one embodiment, Hy is bound to Q′ via a covalent bond with a carboxyl function of Hy carried by GpR, GpG, GpA, GpH, GpL or GpC to form an amide bond.

In one embodiment, Hy is bound to Q′ via a covalent bond with an amine function of Hy carried by GpR, GpG, GpA, GpL or GpH to form an amide bond.

In one embodiment, the PLG chains are bound to Q′ via a covalent bond between Fc, Fc”, Fb, Fb′, Fa or Fa′ and the carbonyl function in the C terminal position of the PLG chain, thus forming an amide bond.

In one embodiment, the PLG chains are bound to Q′ via a covalent bond between Fc, Fc”, Fb, Fb′, Fa or Fa′ and the function amine in the N terminal position of the PLG chain, thus forming an amide bond.

In one embodiment Q′ is a radical according to formula V and q=1.

In one embodiment Q′ is a radical according to formula V bound to one or two radicals according to formula III and 23.

In one embodiment Q′ is a radical according to formula V bound to one or two radicals according to formula IV and 23.

In one embodiment Q′ is a radical according to formula V bound to a radical according to formula III and q=2.

In one embodiment q Q′ are chosen in the group consisting of the radicals according to formula III, IV or V and Q comprises at least two radicals according to formula V, with 2 q 4 and Q[-*]i is a radical in which i=4 and said copolyamino acid according to formula I is a copolyamino acid according to general formula I:

Q[Hy]ᵢ[PLG]ₖ Formula I
With j=2 and k=2
the 2 Hy according to formula X, bound to Q′ via a covalent bond with Fa, Fa′, Fb, Fb′ or Fc, Fc′, Fc″ forming an amide bond,
the 2 PLG chains being bound to Q′ via a covalent bond with Fa, Fa′, Fb, Fb′ or Fc, Fc′, Fc″, thus forming an amide bond.

In one embodiment, the 2 Hy being according to formula X, bound to Q′ via a covalent bond with Fc′, forming an amide bond, and the 2 PLG chains being bound to Q′ via a covalent bond with Fc″, thus forming an amide bond.

In one embodiment, q=2.

In one embodiment, q=3

In one embodiment, q=4.

In one embodiment, Fc is —CO— and Fa, Fa′, Fb and Fb′ are —NH—.

In one embodiment, Fc′ is —NH— and Hy is bound to Fc′ by the carbonyl function carried by GpR, GpG, GpA GpH, GpC or GpL de Hy.

In one embodiment, Fc″ is —NH— and PLG is bound to Fc″ by the carbonyl function in the C terminal position of the PLG.

In one embodiment Q is a radical consisting of radicals chosen among radicals according to formula IV or V with at least two radicals according to formula V, with 2≤q≤3 and Q[-]i is a radical in which i=4.

In one embodiment Q is a radical consisting of radicals chosen among radicals according to formula III or V with at least two radicals according to formula V, with 2≤q≤3 and Q[-]i is a radical in which i=4.

In one embodiment Q is a radical consisting of radicals chosen among radicals according to formula V with at least two radicals according to formula V, with 3 and Q[-]i is a radical according to formula in which i=4.

In one embodiment q Q′ are chosen in the group consisting of the radicals according to formula VI and III and Q comprises at least two radicals according to formula VI, with 2≤q≤3 and Q[-*]i is a radical in which i=4 and said copolyamino acid according to formula I is a copolyamino acid according to general formula I:

Q[HY]ⱼ[PLG]ₖ Formula I
With j=2 and k=2
the 2 Hy being according to formula X, bound to Q′ via a covalent bond with Fy, forming an amide bond,
the 2 PLG chains being bound to Q′ via a covalent bond with Fd or Fd′, thus forming an amide bond.

In one embodiment q Q′ are chosen in the group consisting of the radicals according to formula VI and III and Q comprises at least two radicals according to formula VI, with q=3 and Q[-*]i is a radical in which i=4.

In one embodiment q Q′ are chosen in the group consisting of radicals according to formula III, IV, V or VI with at least two radicals chosen among radicals according to formula V and radicals according to formula VI, with 2 q 5 and Q[-*]i is a radical in which 4 i 6

and said copolyamino acid is a copolyamino acid according to general formula I:

Q[HY]ⱼ[PLG]ₖ Formula I
With j=1 and 3≤k≤5
Hy according to Formula X is bound to Q′ via a covalent bond with Fa, Fb, Fc or Fy, forming an amide bond,
the PLG k chains being bound to Fa, Fa′, Fb, Fb′, Fc″, Fd or Fd′ by a covalent bond, thus forming an amide bond.

In one embodiment q Q′ are chosen in the group of radicals according to formula III, IV, V or VI with at least two radicals chosen among radicals according to formula V and radicals according to formula VI, with 2 q 3 and Q[-*]i is a radical in which i=4 and said copolyamino acid is a copolyamino acid according to general formula I:

Q[HY]ⱼ[PLG]ₖ Formula I
With j=1 and k3
Hy being according to Formula X, bound to Q′ via a covalent bond with Fc, forming an amide bond,
the 3 PLG chains being bound to Fc″, Fd, Fd′ by a covalent bond, thus forming an amide bond.

In one embodiment, Q is a radical consisting of at least a radical according to formula VI and at least a radical according to formula V with q≥2 and Q[-*]ᵢ is a radical in which i=4

In one embodiment, with j=1 and k=3

Fc′ with Fc′=—CO— is bound to Fy by a covalent bond to form an amide bond
Fc″ is bound at a PLG chain by a covalent bond to form an amide bond.

In one embodiment, Fc″ is —NH— and is bound to the PLG by the carbonyl in the C terminal position to form an amide bond.

In one embodiment, Fc is —NH— and is bound to the carbonyl of Hy carried by GpR, GpA, GpG, GpH, GpL or GpC.

In one embodiment said hydrophobic radical -Hy is chosen among radicals according to formula X as defined hereafter:

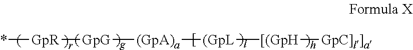

wherein
GpR is chosen among radicals according to formula VII, VII′ or VII″:

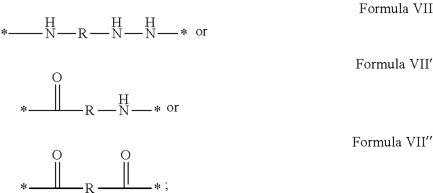

GpG and GpH identical or different are chosen among radicals according to formula XI or XI′:

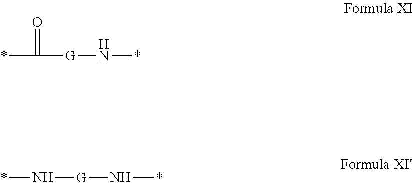

GpA is chosen among radicals according to formula VIII

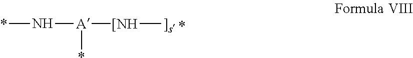

Wherein A′ is chosen among radicals according to formula VIII′, VIII″ or VIII′″

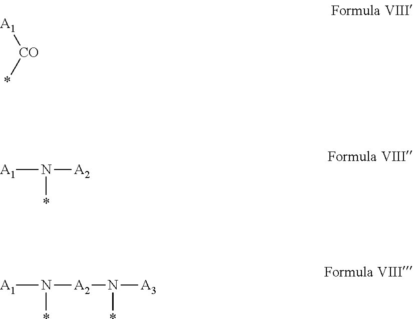

-GpL is chosen among radicals according to formula XII

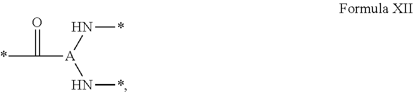

GpC is a radical according to formula IX:

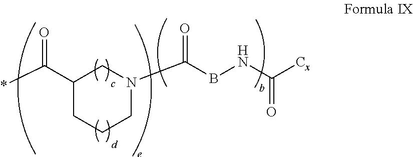

the * indicate the binding sites of the different groups bound by amide functions;
a is an integer equal to 0 or to 1 and a′=1 if a=0 and a′=1, 2 or 3 if a=1;
a′ is an integer equal to 1, to 2 or to 3
b is an integer equal to 0 or to 1;
c is an integer equal to 0 or to 1, and if c is equal to 0 then d is equal to 1 or to 2;
d is an integer equal to 0, to 1 or to 2;
e is an integer equal to 0 or to 1;
g is an integer equal to 0, to 1, to 2, to 3 to 4 to 5 or to 6;
h is an integer equal to 0, to 1, to 2, to 3 to 4 to 5 or to 6;
l is an integer equal to 0 or 1 and l′=1 if l=0 and l′=2 if l=1;
r is an integer equal to 0 or to 1, and
s′ is an integer equal to 0 or 1;
A, A₁, A₂ and A₃ identical or different are linear or branched alkyl radicals comprising from 1 to 6 carbon atoms;
B is a linear or branched alkyl radical, optionally comprising an aromatic ring, comprising from 1 to 9 carbon atoms;
Cₓ is a monovalent linear or branched alkyl radical, in which x indicates the number of carbon atoms and:
   When the hydrophobic radical -Hy carries 1 -GpC, then 9≤x≤25,
   When the hydrophobic radical -Hy carries 2 -GpC, then 9≤x≤15,
   When the hydrophobic radical -Hy carries 3 -GpC, then 7≤x≤13,
   When the hydrophobic radical -Hy carries 4 -GpC, then 7≤x≤11
   When the hydrophobic radical -Hy carries at least 5 -GpC then, 6≤x≤11,
G is a branched alkyl radical from 1 to 8 carbon atoms said alkyl radical bearing one or several carboxylic function(s).
H is a branched alkyl radical from 1 to 8 carbon atoms said alkyl radical bearing one or several carboxylic function(s),
R is a radical chosen in the group consisting of a divalent linear or branched alkyl radical, comprising from 1 to 12 carbon atoms, a divalent linear or branched alkyl radical, comprising from 1 to 12 carbon atoms bearing one or several —CONH₂ functions or a non-substituted ether or polyether radical comprising from 4 to 14 carbon atoms and from 1 to 5 oxygen atoms:
Hydrophobic radical(s) -Hy according to formula X being bound to Q:
   via a covalent bond between a carbonyl of the hydrophobic radical -Hy and a nitrogen atom carried by Q thus forming an amide function from the reaction of an amine function carried by the precursor of Q and an acid function carried by the precursor Hy′ of the hydrophobic radical -Hy, and
   via a covalent bond between a nitrogen atom of the hydrophobic radical -Hy and a carbonyl carried by Q, thus forming an amide function from the reaction of an amine function of the precursor Hy′ of the hydrophobic radical -Hy and an acid function carried by the precursor of the radical Q,
the ratio M between the number of hydrophobic radicals and the number of glutamic or aspartic units being comprised from 0<M≤0.5;
when several hydrophobic radicals are carried by a copolyamino acid then they are identical or different,
the degree of polymerization DP of glutamic or aspartic units of the PLG chains is comprised from 5 and 250;
the free carboxylic acid functions being in the form of alkaline cation chosen in the group consisting of Na⁺ and K⁺.

In one embodiment, said at least a hydrophobic radical -Hy is chosen among radicals according to formula X wherein
l=0,
according to formula Xd as defined hereafter

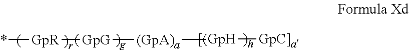

wherein
GpR is chosen among radicals according to formula VII, VII′ or VII″:

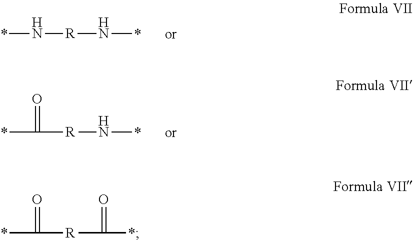

GpG is chosen among radicals according to formula XI or XI′:

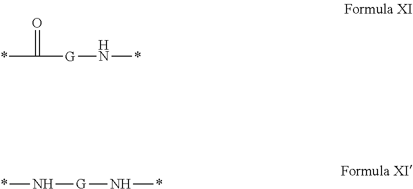

GpA is chosen among radicals according to formula VIII wherein s′=1 is according to formula VIIIa or according to formula VIII wherein s′=0 is according to formula VIIIb:

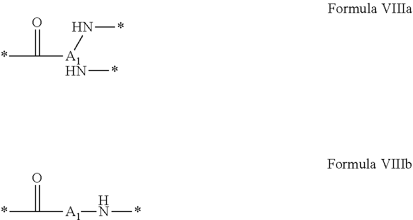

GpC is a radical according to formula IX:

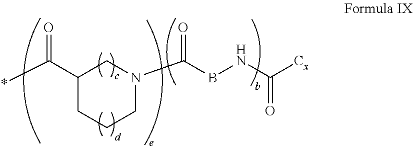

the * indicate the binding sites of the different groups bound by amide functions;
a is an integer equal to 0 or to 1 and a′=1 if a=0 and a′=1 or a′=2 or a′=3 if a=1;
a′ is an integer equal to 1 or 2 and
   if a′ is equal to 1 then a is equal to 0 or to 1 and GpA is a radical according to formula VIIIb and,
   if a′ is equal to 2 then a is equal to 1, and GpA is a radical according to formula VIIIa;
b is an integer equal to 0 or to 1;
c is an integer equal to 0 or to 1, and if c is equal to 0 then d is equal to 1 or to 2;
d is an integer equal to 0, to 1 or to 2;
e is an integer equal to 0 or to 1;
g is an integer equal to 0, to 1, to 2, to 3 to 4 to 5 or to 6;
h is an integer equal to 0, to 1, to 2, to 3 to 4 to 5 or to 6;
r is an integer equal to 0 or to 1, and
s′ is an integer equal to 0 or 1;
A₁ is a linear or branched alkyl radical comprising from 1 to 6 carbon atoms;
B is a linear or branched alkyl radical, optionally comprising an aromatic ring, comprising from 1 to 9 carbon atoms;
Cₓ is a monovalent linear or branched alkyl radical, in which x indicates the number of carbon atoms and:
   When the hydrophobic radical -Hy carries 1 -GpC, then 9≤x≤25,
   When the hydrophobic radical -Hy carries 2 -GpC, then 9≤x≤15,
   When the hydrophobic radical -Hy carries 3 -GpC, then 7≤x≤13,
   When the hydrophobic radical -Hy carries 4 -GpC, then 7≤x≤11
   When the hydrophobic radical -Hy carries at least 5 -GpC then, 6≤x≤11,
G is a branched alkyl radical from 1 to 8 carbon atoms said alkyl radical bearing one or several carboxylic function(s),
H is a branched alkyl radical from 1 to 8 carbon atoms said alkyl radical bearing one or several carboxylic function(s),
R is a radical chosen in the group consisting of a divalent linear or branched alkyl radical, comprising from 1 to 12 carbon atoms, a divalent linear or branched alkyl radical, comprising from 1 to 12 carbon atoms bearing one or several functions —CONH₂ or a non-substituted ether or polyether radical comprising from 4 to 14 carbon atoms and from 1 to 5 oxygen atoms:
Hydrophobic radical(s) Hy according to formula X being bound to Q:
   via a covalent bond between a carbonyl of the hydrophobic radical and a nitrogen atom carried by Q thus forming an amide function from the reaction of an amine function carried by the precursor of Q and an acid function carried by the precursor of the hydrophobic radical, and
   via a covalent bond between a nitrogen atom of the hydrophobic radical and a carbonyl carried by Q, thus forming an amide function from the reaction of an amine function of the precursor -Hy′ of the hydrophobic radical and an acid function carried by the precursor of the radical Q.
the ratio M between the number of hydrophobic radicals and the number of glutamic or aspartic units being comprised from 0<M≤0.5;
when several hydrophobic radicals are carried by a copolyamino acid then they are identical or different,
the degree of polymerization DP of glutamic or aspartic units of the PLG chains is comprised from 5 and 250;
the free carboxylic acid functions being in the form of alkaline cation chosen in the group consisting of Na⁺ and K⁺.

In one embodiment said hydrophobic radical -Hy is chosen among radicals according to formula X as defined hereafter wherein l=0,
GpA is chosen among radicals according to formula VIII wherein s′=1 and A′ chosen among radicals according to formula VIII″ or VIII′″:

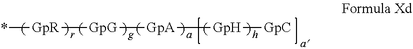

wherein
GpR is chosen among radicals according to formula VII, VII′ or VII″:

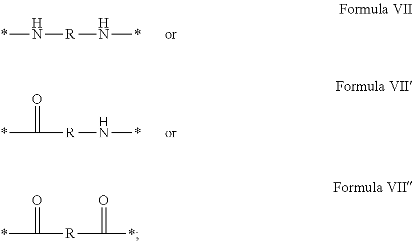

GpG is chosen among radicals according to formula XI or XI′:

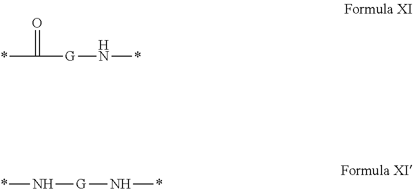

GpA is chosen among radicals according to formula, VIIIc or VIIId:

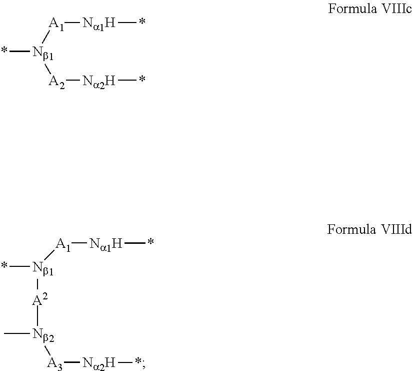

GpC is a radical according to formula IX:

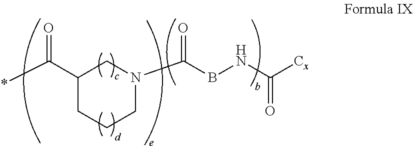

the * indicate the binding sites of the different groups bound by amide functions;
a is an integer equal to 0 or to 1 and a′=1 if a=0 and a′=2 if a=1;
a′ is an integer equal to 2 or to 3 and
   if a′ is equal to 1 then a is equal to 0 and,
   if a′ is equal to 2 or 3 then a is equal to 1, and GpA is a radical according to formula VIIIc or VIIId;
b is an integer equal to 0 or to 1;
c is an integer equal to 0 or to 1, and if c is equal to 0 then d is equal to 1 or to 2;
d is an integer equal to 0, to 1 or to 2;
e is an integer equal to 0 or to 1;
g is an integer equal to 0, to 1, to 2, to 3 to 4 to 5 or to 6;
h is an integer equal to 0, to 1, to 2, to 3 to 4 to 5 or to 6;
r is an integer equal to 0 or to 1, and
s′ is an integer equal to 1;
A₁, A₂, A₃ identical or different are linear or branched alkyl radicals comprising from 1 to 6 carbon atoms;
B is a linear or branched alkyl radical, optionally comprising an aromatic ring, comprising from 1 to 9 carbon atoms;
Cₓ is a monovalent linear or branched alkyl radical, in which x indicates the number of carbon atoms and:
   When the hydrophobic radical -Hy carries 1 -GpC, then 9≤x≤25,
   When the hydrophobic radical -Hy carries 2 -GpC, then 9≤x≤15,
   When the hydrophobic radical -Hy carries 3 -GpC, then 7≤x≤13,
   When the hydrophobic radical -Hy carries 4 -GpC, then 7≤x≤11
   When the hydrophobic radical -Hy carries at least 5 -GpC alors, 6≤x≤11,
the hydrophobic radical(s) Hy according to formula X being bound to Q:
   via a covalent bond between a carbonyl of the hydrophobic radical and a nitrogen atom carried by Q thus forming an amide function from the reaction of an amine function carried by the precursor of Q and an acid function carried by the precursor Hy′ of the hydrophobic radical, and
   via a covalent bond between a nitrogen atom of the hydrophobic radical and a carbonyl carried by Q, thus forming an amide function from the reaction of an amine function of the precursor Hy′ of the hydrophobic radical and an acid function carried by the precursor of the radical Q.
G is a branched alkyl radical from 1 to 8 carbon atoms said alkyl radical bearing one or several carboxylic function(s),
H is a branched alkyl radical from 1 to 8 carbon atoms said alkyl radical bearing one or several carboxylic function(s),
R is a radical chosen in the group consisting of a divalent linear or branched alkyl radical, comprising from 1 to 12 carbon atoms, a divalent linear or branched alkyl radical, comprising from 1 to 12 carbon atoms bearing one or several functions —CONH₂ or a non-substituted ether or polyether radical comprising from 4 to 14 carbon atoms and from 1 to 5 oxygen atoms:
the ratio M between the number of hydrophobic radicals and the number of glutamic or aspartic units being comprised from 0<≤0.5;
when several hydrophobic radicals are carried by a copolyamino acid then they are identical or different,
the degree of polymerization DP of glutamic or aspartic units of the PLG chains is comprised from 5 and 250;
the free carboxylic acid functions being in the form of alkaline cation chosen in the group consisting of Na⁺ and K⁺.

In one embodiment, R is a radical chosen in the group consisting of:
a divalent linear or branched alkyl radical, comprising if GpR is a radical according to formula VII from 2 to 12 carbon atoms or if GpR is a radical according to formula VII′ from 1 to 11 carbon atoms;
a divalent linear or branched alkyl radical, comprising if GpR is a radical according to formula VII from 2 to 11 carbon atoms or if GpR is a radical according to formula VII′ from 1 to 11 carbon atoms, said alkyl radical bearing one or several functions —CONH₂, and
a non-substituted ether or polyether radical comprising from 4 to 14 carbon atoms and from 1 to 5 oxygen atoms.

In formulas, the * indicate the binding sites of hydrophobic radicals to Q[-*]ᵢ. Radicals -Hy are bound to Q[-*]ᵢ via amide functions.

In formulas VII, VII′ and VII″, the * indicate, from left to right respectively, the binding sites of GpR:
to Q[-*]ᵢ et
to GpG if g=1 or to GpA if g=0.

In formulas VIIIa, VIIIb, VIIIc and VIIId, the * indicate, from left to right respectively, the binding sites of GpA:
to GpG if g=1 or to GpR if r=1 and g=0 or to Q[-*], if g=r=0 and
to GpL if l=1 or to GpH if h=1 and l=0 or to GpC if l=h=0

In the formula IX, the * indicate the binding sites of GpC:
to GpH if h=1,
to GpL if l=1 and h=0
to GpA if a=1 and h=l=0
to GpG if g=1 and h=l=a=0
to GpR if r=1 and h=l=a=g=0
to Q[-*]ᵢ if h=l=a=g=r=0
All attachments between the different groups GpR, GpG, GpA, GpL, GpH and GpC are amide functions.

Radicals Hy, GpR, GpG, GpA, GpL, GpH and GpC are each independently identical or different from one residue to an other.

In one embodiment, r=0 and the hydrophobic radical according to formula X is bound to Q via a covalent bond between a carbonyl of the hydrophobic radical and a nitrogen atom carried by Q thus forming an amide function from the reaction of an amine function carried by the precursor of Q and an acid function carried by the precursor Hy′ of the hydrophobic radical.

In one embodiment, r=1 and the hydrophobic radical according to formula X is bound to Q:
via a covalent bond between a nitrogen atom of the hydrophobic radical and a carbonyl carried by Q, thus forming an amide function from the reaction of an amine function of the precursor Hy′ of the hydrophobic radical and an acid function carried by the precursor of the radical Q or,
via a covalent bond between a carbonyl of the hydrophobic radical and a nitrogen atom carried by Q, thus forming an amide function from the reaction of an acid function of the precursor Hy′ of the hydrophobic radical -Hy and an amine function of the precursor Q′ of the radical Q.

In one embodiment, if GpA is a radical according to formula VIIIc and r=1, then:
the GpL or GpH (if l=0) or GpC (if l=h=0) are bound to N_{α1} and N_{α2} and Q[-*]i is bound via -GpR-GpG- or -GpR- (if g=0) to N_{β1}, or
the GpL or GpH (if l=0) or GpC (if l=h=0) are bound to N_{α1} and N_{β1}, and Q[-*]i is bound via -GpR-GpG- or -GpR- (if g=0) to N_{α2}; or
the GpL or GpH (if l=0) or GpC (if l=h=0) are bound to N_{α2} and N_{β1}, and Q[-*]i is bound via -GpR-GpG- or -GpR- (if g=0) to N_{α1}.

In one embodiment, if GpA is a radical according to formula VIIIc and r=0, then:
the GpC are bound to N_{α1} and N_{α2} and Q[-*]i (if g=0) or GpG is bound to N_{β1}; or
the GpC are bound to N_{α1} and N_{β1}, and Q[-*]i (if g=0) or GpG is bound to N_{α2}; or
the GpC are bound to N_{α2} and N_{β1}, and Q[-*]i (if g=0) or GpG is bound to N_{α1}.

In one embodiment, if GpA is a radical according to formula VIIId and r=1, then
the GpL or GpH (if l=0) or GpC (if l=h=0) are bound to N_{α1}, N_{α2} and N_{β1} and Q[-*]i is bound via -GpR-GpG- or -GpR- (if g=0) to N_{β2}; or
the GpL or GpH (if l=0) or GpC (if l=h=0) are bound to N_{α1}, N_{α2} and N_{β2} and Q[-*]i is bound via -GpR-GpG- or -GpR- (if g=0) to N_{β1}; or
the GpL or GpH (if l=0) or GpC (if l=h=0) are bound to N_{α1}, N_{β1} and N_{β2} and Q[-*]i is bound via -GpR-GpG- or -GpR- (if g=0) to N_{α2}; or
the GpL or GpH (if l=0) or GpC (if l=h=0) are bound to N_{α2}, N_{β1} and N_{β2} and Q[-*]i is bound via -GpR-GpG- or -GpR- (if g=0) to N_{α1}.

In one embodiment, if GpA is a radical according to formula VIIId and r=0, then
the GpC are bound to N_{α1}, N_{α2} and N_{β1} and Q[-*]i (if g=0) or GpG is bound to N_{β2}; or
the GpC are bound to N_{α1}, N_{α2} and N_{β2} and Q[-*]i (if g=0) or GpG is bound to N_{β1}; or
the GpC are bound to N_{α1}, N_{β1} and N_{β2} and Q[-*]i (if g=0) or GpG is bound to N_{α2}; or
the GpC are bound to N_{α2}, N_{β1} and N_{β2} and Q[-*]i (if g=0) or GpG is bound to

Nₐₗ

In one embodiment, when a′=1, x is comprised from 11 and 25 (11≤x≤25). In particular, when x is comprised from 15 and 16 (x=15 or 16) then r=1 and R is an ether radical or polyether and when x is greater than 17 (x≥17) then r=1 and R is an ether radical or polyether.

In one embodiment, when a′=2, x is comprised from 9 and 15 (9≤x≤15).

In one embodiment, said at least a hydrophobic radical -Hy is chosen among radicals according to formula X wherein a=1 and a′=1 according to formula Xa as defined hereafter:

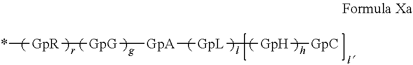

Wherein GpA is a radical according to formula VIII and A′ is chosen among radicals according to formula VIII′ with s′=0 and GpA is a radical according to formula VIIIb

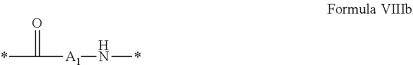

And GpR, GpG, GpA, GpL, GpH, GpC, A₁, r, g, h, l and l′ are as defined above.

In one embodiment, said at least a hydrophobic radical -Hy is chosen among radicals according to formula X wherein a=1 according to formula Xb as defined hereafter:

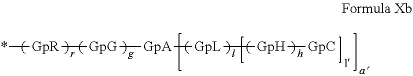

Wherein GpA is a radical according to formula VIII and A′ is chosen among radicals according to formula VIII′ with s′=1 and GpA is a radical according to formula VIIIa

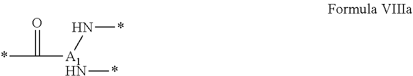

And GpR, GpG, GpA, GpL, GpH, GpC, A₁, a′, r, g, h, l and l′ are as defined above.

In one embodiment, said hydrophobic radical -Hy is chosen among radicals according to formula X wherein a=1 as defined hereafter:

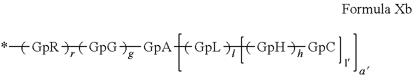

Wherein GpA is a radical according to formula VIII and A is chosen among radicals according to formula VIII″ with s′=1 and GpA is a radical according to formula VIIIc

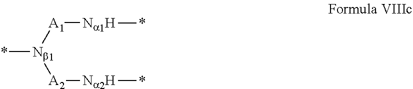

And GpR, GpG, GpA, GpL, GpH, GpC, A₁, A₂, r, g, h, a′, l and l′ are as defined above.

In one embodiment, said at least a hydrophobic radical -Hy is chosen among radicals according to formula X wherein a=1 as defined hereafter:

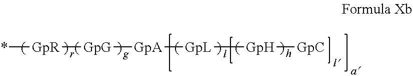

Wherein GpA is a radical according to formula VIII and A is chosen among radicals according to formula VIII′″ with s′=1, and GpA is a radical according to formula VIIId

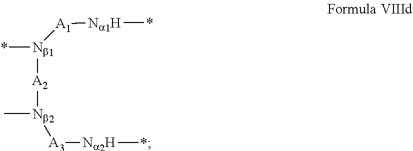

And GpR, GpG, GpA, GpL, GpH, GpC, A₁, A₂, A₃, a′, r, g, h, l and l′ are as defined above.

In one embodiment, said at least a hydrophobic radical -Hy is chosen among radicals according to formula X wherein r=1 according to formula Xc, as defined hereafter:

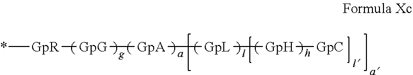

Wherein GpR is a radical according to formula VII.

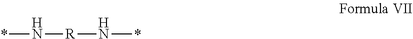

And GpR, GpG, GpA, GpL, GpH, GpC, R, a, a′, g, h, l and l′ are as defined above.

In one embodiment, said at least a hydrophobic radical -Hy is chosen among radicals according to formula Xc as defined hereafter:

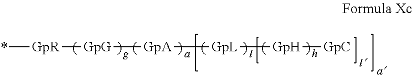

Wherein GpR is a radical according to formula VII′.

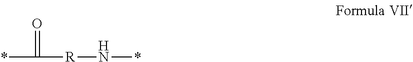

In one embodiment, said at least a hydrophobic radical -Hy is chosen among radicals according to formula Xc as defined hereafter:

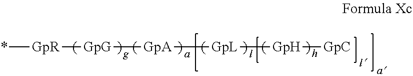

Wherein GpR is a radical according to formula VII″.

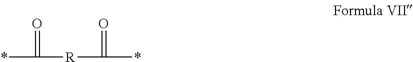

In one embodiment said at least a hydrophobic radical -Hy is chosen among radicals according to formula X as defined hereafter:

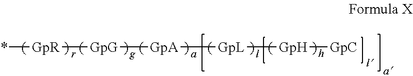

Wherein GpC is a radical according to formula IX wherein e=0 and GpC is a radical according to formula IXa

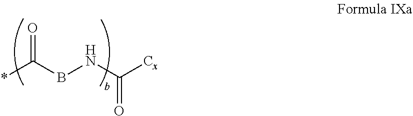

In one embodiment said at least a hydrophobic radical -Hy is chosen among radicals according to formula X as defined hereafter:

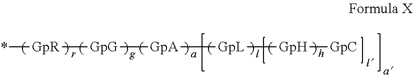

Wherein GpC is a radical according to formula IX wherein e=1, b=0 and GpC is a radical according to formula IXd

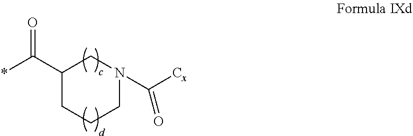

In one embodiment said at least a hydrophobic radical -Hy is chosen among radicals according to formula X as defined hereafter:

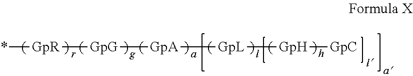

Wherein GpC is a radical according to formula IX dansl laquelle e=1 and GpC is a radical according to formula IXb

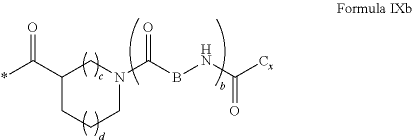

In one embodiment said at least a hydrophobic radical -Hy is chosen among radicals according to formula X wherein r, g, a, I, h are equal to 0, according to formula Xd as defined hereafter:

*-GPC Formula Xd′.

In one embodiment said at least a hydrophobic radical -Hy is chosen among radicals according to formula X wherein r, g, a, l, h are equal to 0, according to formula Xd′ as defined hereafter:

*-GPC Formula Xd′

Wherein GpC is a radical according to formula IX Wherein e=0, b=0 and GpC is a radical according to formula IXc

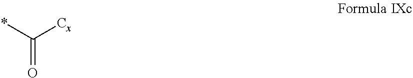

In one embodiment, the composition according to the invention is characterized in that said hydrophobic radicals are chosen among hydrophobic radicals according to formula X wherein GpA is a radical according to formula VIIIb, a′=1 and l=0 according to the following formula Xe:

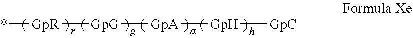

GpR, GpG, GpA, GpH, GpC, r, g, h, and a are as defined above.

In one embodiment, the composition according to the invention is characterized in that said hydrophobic radicals are chosen among hydrophobic radicals according to formula X wherein, a′=2 and a=1 and l=0 according to the following formula Xf:

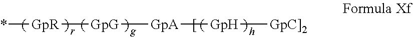

GpR, GpG, GpA, GpH, GpC, r, g and h are as defined above.

In one embodiment, the composition according to the invention is characterized in that said hydrophobic radicals are chosen among hydrophobic radicals according to formula X wherein h=0, l=0 and I′=1 according to the following formula Xg:

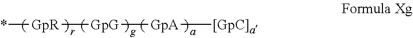

GpR, GpG, GpA, GpC, r, g, a and a′ are as defined above.

In one embodiment, the composition according to the invention is characterized in that said hydrophobic radicals are chosen among hydrophobic radicals according to formula X wherein h=0, a′=1 according to the following formula Xh:

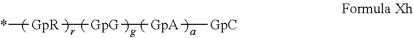

GpR, GpG, GpA, GpC, r, a and g are as defined above.

In one embodiment, the composition according to the invention is characterized in that said hydrophobic radicals are chosen among hydrophobic radicals according to formula X wherein h=0, a′=2 and a=1 according to the following formula Xi:

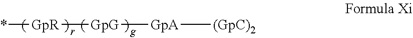

GpR, GpG, GpA, GpC, r and g are as defined above.

In one embodiment, the composition according to the invention is characterized in that said hydrophobic radicals are chosen among hydrophobic radicals according to formula X wherein h=0 and g=0, according to the following formula Xj:

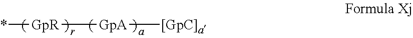

GpR, GpA, GpC, r, a′ and a are as defined above.

In one embodiment, the composition according to the invention is characterized in that said hydrophobic radicals are chosen among hydrophobic radicals according to formula X wherein h=0 and g=0 and a′=1, according to the following formula Xk:

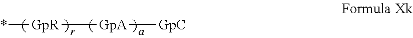

GpR, GpA, GpC, r and a are as defined above.

In one embodiment, the composition according to the invention is characterized in that said hydrophobic radicals according to formula X are chosen among hydrophobic radicals according to formula X wherein h=0 and g=0 and a=1 and a′=2, according to the following formula XI:

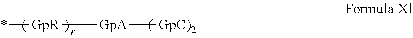

Wherein GpR, GpA, GpC and r are as defined above.

In one embodiment, the composition according to the invention is characterized in that said hydrophobic radicals according to formula X are chosen among hydrophobic radicals according to formula X wherein a=1 and a′=1 and g=l=0,

according to the following formula Xn:

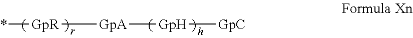

In one embodiment, the composition according to the invention is characterized in that said hydrophobic radicals according to formula X are chosen among hydrophobic radicals according to formula X wherein a=1 and a′=2 and g=l=0, according to the following formula Xp:

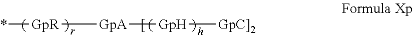

In one embodiment, the composition according to the invention is characterized in that said hydrophobic radicals according to formula X are chosen among hydrophobic radicals according to formula X wherein a=1, g, h and l=0 and a′=3, according to the following formula Xm:

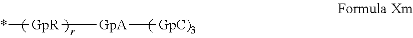

Wherein GpA is a radical chosen among radicals according to formula VIIId and GpR, GpC, r are as defined above.

In one embodiment, a=0,

In one embodiment h=1 and g=0,

In one embodiment h=0 and g=1,

In one embodiment q Q′ are chosen in the group consisting of the radicals according to formula VI, III and IV and Q comprises at least a radical according to formula VI with q≥1 and Q[-*]i is a radical in which i=3

and said copolyamino acid is a copolyamino acid according to general formula I:

Q[HY]ⱼ[PLG]ₖ Formula I

with j=1 and k=2.

In one embodiment q Q′ are chosen in the group consisting of the radicals according to formula III, IV and V and Q comprises at least a radical according to formula V with q≥1 and Q[-*]i is a radical in which i=3

and said copolyamino acid is a copolyamino acid according to general formula I:

Q[Hy]ⱼ[PLG]ₖ Formula I

with j=1 and k=2.

In one embodiment q Q′ are chosen in the group consisting of the radicals according to formula III, IV or V and Q comprises at least two radicals according to formula V, with 2≤q≤3 and Q[-*]i is a radical in which i=4 and said co-polyamino acid is a copolyamino acid according to general formula I:

Q[Hy]ᵢ[PLG]ₖ Formula I

With j=2 and k=2.

In one embodiment q Q′ are chosen in the group of radicals according to formula III, IV, V or VI with at least a radical according to formula V and at least a radical according to formula VI, with 2≤q≤3 and Q[-*]i is a radical in which 4≤i≤6

and said copolyamino acid is a copolyamino acid according to general formula I:

Q[Hy]ᵢ[PLG]ₖ Formula I

With j=1 and 3≤k≤5

In one embodiment, r=0, g=1 and h=0.

In one embodiment, r=1 and GpR is chosen among radicals according to formula VII′ or VII″ and h=0.

In one embodiment, r=1, g=0 and GpR is a radical according to formula VII′ and h=0.

In one embodiment, r=1, g=0 and GpR is a radical according to formula VII′ and h=1.

In one embodiment, r=1, g=0, GpR is a radical according to formula VII′, GpA is chosen among radicals according to formula VIIIa or VIIIb and h=0.

In one embodiment, r=1, g=0, GpR is a radical according to formula VII′, GpA is chosen among radicals according to formula VIIIa or VIIIb and h=1.

In one embodiment, r=1, g=0, GpR is a radical according to formula VII′, GpA is a radical according to formula VIIIa and h=0.

In one embodiment, r=1, g=0, GpR is a radical according to formula VII′, GpA is a radical according to formula VIIIa and h=1.

In one embodiment, r=1, g=0, GpR is a radical according to formula VII′, GpA is a radical according to formula VIIIb and h=0.

In one embodiment, r=1, g=0, GpR is a radical according to formula VII′, GpA is a radical according to formula VIIIb and h=1.

In one embodiment, r=0, and GpA is chosen among radicals according to formula VIIIa and VIIIb.

In one embodiment, r=0, g=0 and GpA is chosen among radicals according to formula VIIIa and VIIIb.

In one embodiment, r=0, GpA is chosen among radicals according to formula VIIIa and VIIIb and h=0

In one embodiment, the composition according to the invention is characterized in that the hydrophobic radical is a radical according to formula X, Xc, Xe, Xd, Xg, Xh Xj or Xk wherein r is equal to 1 (r=1) and a is equal to 0 (a=0).

In one embodiment, the composition according to the invention is characterized in that the hydrophobic radical is a radical according to formula X, Xd, Xd′Xe, Xg, Xh Xj or Xk wherein r is equal to 0 (r=0) and a is equal to 0 (a=0).

In one embodiment, the composition according to the invention is characterized in that the hydrophobic radical is a radical according to formula X, Xa, Xb, Xc, Xd Xe, Xf, Xg, Xh, Xi, Xj, Xk, Xl, Xn, Xp or Xm wherein r is equal to 1 (r=1) and a is equal to 1 (a=1).

In one embodiment, the composition according to the invention is characterized in that the hydrophobic radical is a radical according to formula X, Xa, Xb, Xc, Xd, Xe, Xg, Xf, Xh, Xi, Xj, Xk, Xl; Xn, Xp or Xm wherein r=1 and GpR is a radical according to formula VII.

In one embodiment, the composition according to the invention is characterized in that the hydrophobic radical is a radical according to formula Xa, Xe, Xh, Xk or Xl, wherein r=1 and GpR is a radical according to formula VII.

In one embodiment, the composition according to the invention is characterized in that the hydrophobic radical is a radical according to formula Xf, Xi or Xl wherein r=1 and GpR is a radical according to formula VII.

In one embodiment, the composition according to the invention is characterized in that the hydrophobic radical is a radical according to formula Xm wherein r=1 and GpR is a radical according to formula VII.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII wherein R is a divalent linear alkyl radical comprising from 2 to 12 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII wherein R is a divalent alkyl radical comprising from 2 to 6 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII wherein R is a divalent linear alkyl radical comprising from 2 to 6 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII wherein R is a divalent alkyl radical comprising from 2 to 4 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII wherein R is a divalent linear alkyl radical comprising from 2 to 4 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII wherein R is a divalent alkyl radical comprising 2 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII′.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII′ wherein R is a divalent linear alkyl radical comprising from 1 to 11 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII′ wherein R is a divalent alkyl radical comprising from 1 to 6 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII or VII′, wherein R is a divalent alkyl radical, comprising from 2 to 5 carbon atoms and bearing one or several amide functions (—CONH₂).

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII′ or VII, wherein R is a divalent linear alkyl radical, comprising from 2 to 5 carbon atoms and bearing one or several amide functions (—CONH₂).

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″ wherein R is a radical chosen in the group consisting of radicals represented by the formulas below:

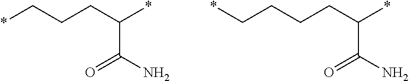 [SMILES: *CCCC(*)C(N)=O ; *CCCCC(*)C(N)=O]

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″, wherein R is a non substituted linear ether or polyether radical comprising from 4 to 14 carbon atoms and from 1 to 5 oxygen atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″, wherein R is an ether radical.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″, wherein R is an ether radical comprising from 4 to 6 carbon atoms.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″ wherein R is an ether radical represented by the Formula

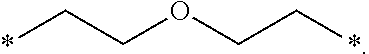

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″, wherein R is a polyether radical.

In one embodiment, the composition according to the invention is characterized in that the hydrophobic radical is a radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″, wherein R is a linear polyether radical comprising from 6 to 10 carbon atoms and from 2 to 3 oxygen atoms.

In one embodiment, the composition according to the invention is characterized in that the hydrophobic radical according to formula X, Xa, Xb, Xc, Xe, Xg, Xf, Xh, Xi, Xj, Xk, Xl, Xd, Xn, Xp or Xm wherein r=1 and GpR is a radical according to formula VII, VII′ or VII″, wherein R is a polyether radical chosen in the group consisting of radicals represented by the formulas below:

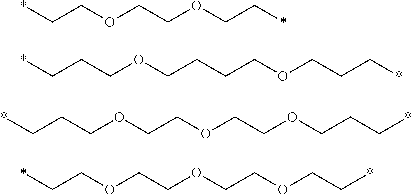

In one embodiment, the composition according to the invention is characterized in that the hydrophobic radical according to formula X, Xa, Xb, Xc, Xe, Xg, Xf, Xh, Xi, Xj, Xk, Xl, Xd, Xn, Xp or Xm wherein r=1 and GpR is a radical, wherein R is a polyether radical chosen in the group consisting of radicals represented by the formulas below:

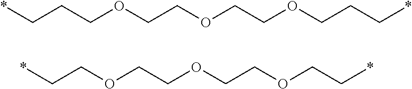

In one embodiment, the composition according to the invention is characterized in that the hydrophobic radical is a radical according to formula X, Xa, Xb, Xc, Xd, Xe, Xg, Xf, Xh or Xi wherein g=1 and GpG is chosen among radicals according to formula XIa, XIb, XIc, XId, XI′e or XI′f represented below.

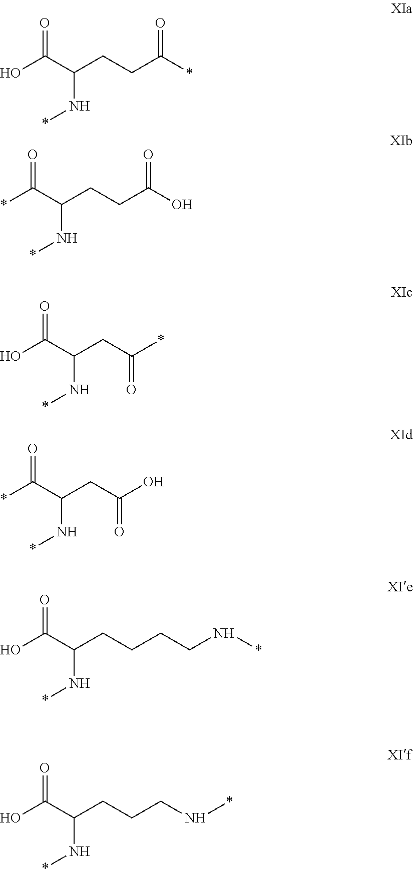

In one embodiment, the composition according to the invention is characterized in that the hydrophobic radical is a radical according to formula X, Xa, Xb, Xc, Xd, Xe or Xf wherein h=1 and GpH is chosen among radicals according to formula XIa, XIb, XIc, XId, XI′e or XI′f represented below.

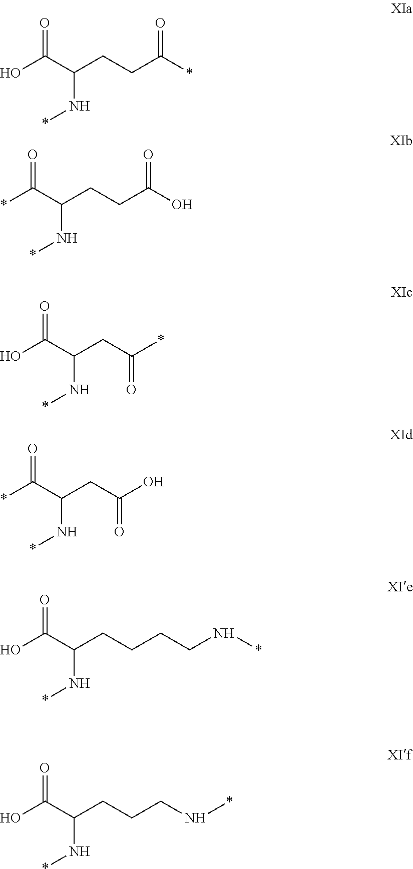

In one embodiment, the composition according to the invention is characterized in that the hydrophobic radical is a radical according to formula according to formula X, Xa, Xb, Xc, Xe, Xg, Xh, Xj, Xk or Xn wherein a is equal to 1 (a=1) and a′=1, the radical GpA is a radical according to formula VIIIb and A₁ is chosen in the group consisting of radicals represented by the formulas below:

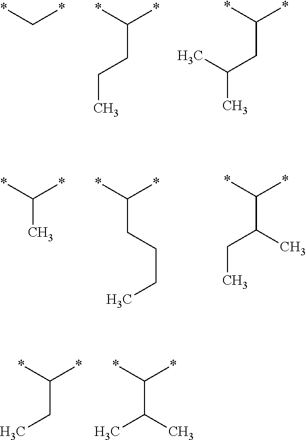

In one embodiment, the composition according to the invention is characterized in that the hydrophobic radical is a radical according to formula X, Xb, Xc, Xg, Xf, Xi, Xj, Xl or Xp wherein the radical GpA according to formula VIIIa is chosen in the group consisting of radicals according to formula VIIIaa and VIIIab represented hereafter:

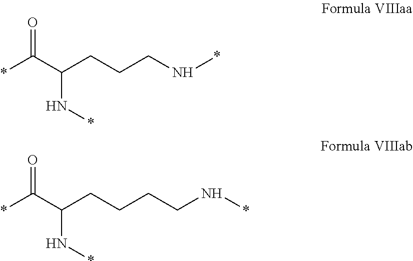 [SMILES: *NCCCC(N*)C(*)=O ; *NCCCCC(N*)C(*)=O]

In one embodiment, the composition according to the invention is characterized in that the hydrophobic radical is a radical according to formula X, Xb, Xc, Xg, Xf, Xi, Xj, Xl or Xp wherein the radical GpA according to formula VIIIa is a radical according to formula VIIIab represented hereafter:

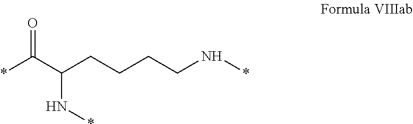 [SMILES: *NCCCCC(N*)C(*)=O]

In one embodiment, the composition according to the invention is characterized in that the hydrophobic radical is a radical according to formula X, Xb, Xc, Xg, Xf, Xi, Xj, Xl or Xp wherein the radical GpA according to formula VIIIc is chosen in the group consisting of radicals in which A₁ and A₂ identical or different, are chosen among linear alkyl radicals.

In one embodiment, the composition according to the invention is characterized in that the hydrophobic radical is a radical according to formula X, Xb, Xc, Xg, Xf, Xi, Xj, Xl or Xp wherein the radical GpA according to formula VIIIc is chosen in the group consisting of radicals in which A₁ and A₂, identical or different, are chosen among linear alkyl radicals comprising from 3 to 4 carbon atoms.

In one embodiment, the composition according to the invention is characterized in that the hydrophobic radical is a radical according to formula X, Xb, Xc, Xg, Xf, Xi, Xj, Xl or Xp wherein the radical GpA according to formula VIIIc is chosen in the group consisting of radicals in which A₁ is chosen among linear alkyl radicals comprising 3 carbon atoms and A₂ is chosen among linear alkyl radicals comprising 4 carbon atoms.

In one embodiment, the composition according to the invention is characterized in that the hydrophobic radical is a radical according to formula X, Xb, Xc, Xg, Xf, Xi, Xj, Xl or Xp wherein the radical GpA according to formula VIIIc is chosen in the group consisting of radicals in which A₁ and A₂, identical are chosen among linear alkyl radicals comprising 3 carbon atoms.

In one embodiment, the composition according to the invention is characterized in that the hydrophobic radical is a radical according to formula X, Xb, Xc, Xg, Xf, Xi, Xj, Xl or Xp wherein the radical GpA according to formula VIIIc is chosen in the group

consisting of radicals VIIIca and VIIIcb:

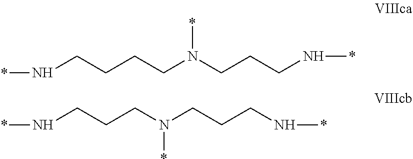

In one embodiment, the composition according to the invention is characterized in that the hydrophobic radical is a radical according to formula X, Xb, Xc, Xg, Xf, Xi, Xj, Xl or Xp wherein the radical GpA according to formula VIIIc is a radical according to formula VIIIca.

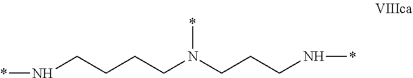 [SMILES: *NCCCCN(*)CCCN*]

In one embodiment, the composition according to the invention is characterized in that the hydrophobic radical is a radical according to formula X, Xb, Xc, Xg, Xf, Xi, Xj, Xl or Xp wherein the radical GpA according to formula VIIIc is a radical according to formula VIIIcb.

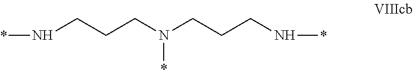 [SMILES: *NCCCN(*)CCCN*]

In one embodiment, the composition according to the invention is characterized in that the hydrophobic radical is a radical according to formula X, Xb, Xc, Xg, Xf, Xi, Xj, Xl or Xp wherein the precursor of the radical GpA according to formula VIIIc is chosen in the group consisting of triamines which are spermidine and norspermidine:

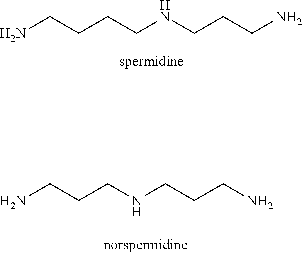 [SMILES: NCCCCNCCCN ; NCCCNCCCN]

In one embodiment, the composition according to the invention is characterized in that the hydrophobic radical is a radical according to formula X, Xb, Xc, Xg, Xf, Xi, Xj, Xl or Xp wherein the precursor of the radical GpA according to formula VIIIc is spermidine.

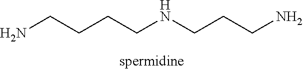

In one embodiment, the composition according to the invention is characterized in that the hydrophobic radical is a radical according to formula X, Xb, Xc, Xg, Xf, Xi, Xj, Xl or Xp wherein the precursor of the radical GpA according to formula VIIIc is norspermidine.

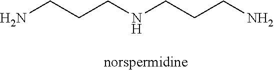

In one embodiment, the composition according to the invention is characterized in that the hydrophobic radical is a radical according to formula Xm wherein the radical GpA is chosen in the group of radicals according to formula VIIId wherein A₁, A₂ and A₃, identical or different, are chosen among linear alkyl radicals comprising from 3 to 4 carbon atoms.

In one embodiment, the composition according to the invention is characterized in that the hydrophobic radical is a radical according to formula Xm wherein the radical GpA according to formula III′ is chosen in the group the group of radicals according to formula VIIId wherein A₁ and A₃ identical are chosen among linear alkyl radicals comprising 3 carbon atoms and A₂ is chosen among linear alkyl radicals comprising 4 carbon atoms.

In one embodiment, the composition according to the invention is characterized in that the hydrophobic radical is a radical according to formula Xm wherein the radical GpA according to formula VIIId is a radical according to formula VIIIda:

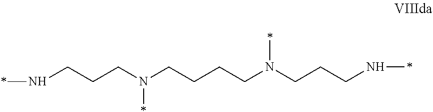 [SMILES: *NCCCN(*)CCCCN(*)CCCN*]

In one embodiment, the composition according to the invention is characterized in that the hydrophobic radical is a radical according to formula Xm wherein the precursor of the radical GpA according to formula VIIId is spermine:

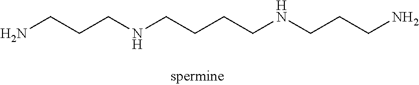 [SMILES: NCCCNCCCCNCCCN]

In one embodiment, the composition according to the invention is characterized in that the hydrophobic radical is a radical according to formula X, Xa, Xb, Xc, Xd, Xd′, Xe, Xf, Xg, Xh, Xi, Xj, Xk, Xl, Xn, Xpou Xm wherein the radical GpC according to formula IX is chosen in the group consisting of radicals according to formula IXa′, IXb′ or IXc′ represented hereafter:

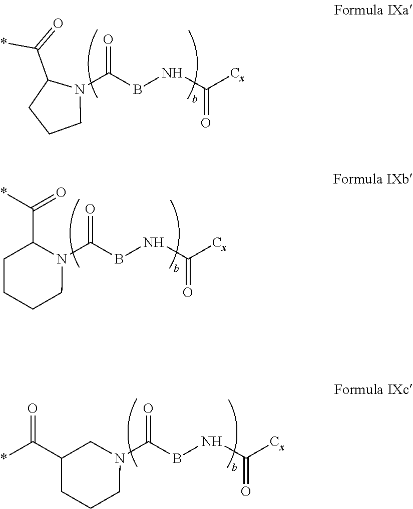

In one embodiment, the composition according to the invention is characterized in that the hydrophobic radical is a radical according to formula X, Xa, Xb, Xc, Xd, Xd′, Xe, Xf, Xg, Xh, Xi, Xj, Xk, Xl, Xn, Xp or Xm wherein the radical GpC is according to formula IXa′.

In one embodiment, the composition according to the invention is characterized in that the hydrophobic radical is a radical according to formula X, Xa, Xb, Xc, Xd, Xe, Xf, Xg, Xh, Xi, Xj, Xk, Xl, Xn, Xp or Xm wherein the radical GpC according to formula IX is chosen in the group consisting of radicals according to formula IXa′, IXb′ or IXc′ in which b is equal to 0, of respectively formulas IXd, IXe, and IXf represented hereafter:

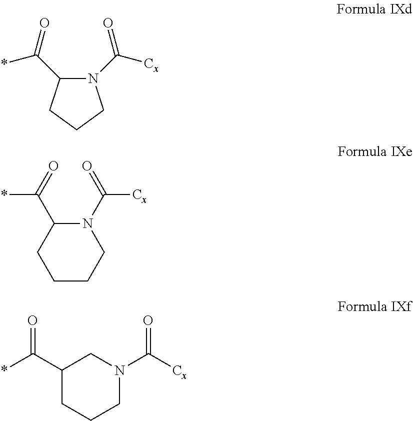

In one embodiment, the composition according to the invention is characterized in that the hydrophobic radical is a radical according to formula X, Xa, Xb, Xc, Xd, Xe, Xf, Xg, Xh, Xi, Xj, Xk, Xl, Xn, Xp or Xm wherein the radical GpC is according to Formula IX or IXa′ in which b=0, and is according to Formula IXd.

In one embodiment, the composition according to the invention is characterized in that the hydrophobic radical is a radical according to formula X, Xa, Xb, Xc, Xd, Xe, Xf, Xg, Xh, Xi, Xj, Xk, Xl, Xn, Xp or Xm wherein the radical GpC according to formula IX wherein b=1 is chosen in the group consisting of radicals in which B is an amino acid residue chosen in the group consisting of radicals represented by the formulas below:

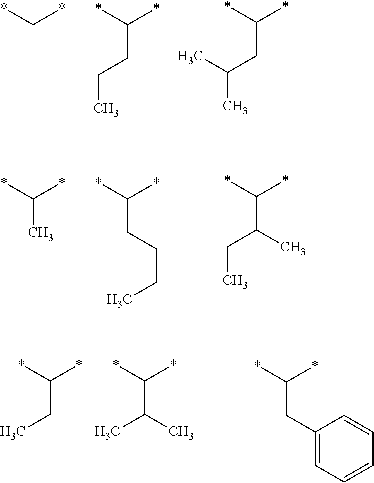

In one embodiment, the composition according to the invention is characterized in that the hydrophobic radical is a radical according to formula X, Xa, Xb, Xc, Xd, Xe, Xf, Xg, Xh, Xi, Xj, Xk, Xl, Xn, Xp or Xm wherein the radical GpC according to formula is according to Formula IX or IXa in which b=1, is chosen in the group consisting of radicals in which B is an amino acid residue chosen in the group consisting of radicals represented by the formulas below:

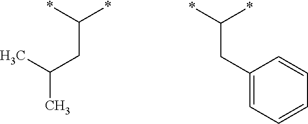

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of linear alkyl radicals comprising from 11 to 25 carbon atoms.

In one embodiment, the composition according to the invention is characterized in that the hydrophobic radical is a radical according to formula X, Xa, Xc, Xe, Xg, Xh, Xj, Xk, Xn wherein a′=1 or l′=1 wherein the radical GpC according to formula IX is chosen in the group consisting of radicals in which Cx is chosen in the group consisting of branched alkyl radicals comprising from 11 to 25 carbon atoms.

In one embodiment, the composition according to the invention is characterized in that the hydrophobic radical is a radical according to formula X, Xa, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals comprising between 11 and 14 carbon atoms.

In one embodiment, the composition according to the invention is characterized in that the hydrophobic radical is a radical according to formula X, Xa, Xc, Xe, Xg, Xh, Xj, Xk, Xn, wherein a′=1 or l′=1 wherein the radical GpC according to formula IX is chosen in the group consisting of radicals in which Cx is chosen in the group consisting of radicals represented by the formulas below:

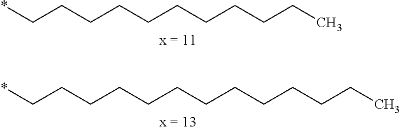 [SMILES: *CCCCCCCCCCC ; *CCCCCCCCCCCCC]

In one embodiment, the composition according to the invention is characterized in that the hydrophobic radical is a radical de X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals comprising between 15 and 16 carbon atoms.

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of radicals represented by the formulas below:

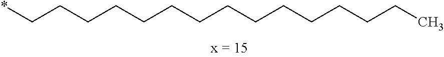 [SMILES: *CCCCCCCCCCCCCCC]

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of radicals represented by the formulas below:

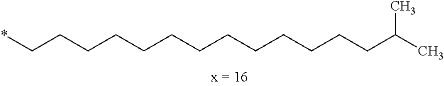 [SMILES: *CCCCCCCCCCCCCC(C)C]

In one embodiment, the composition according to the invention is characterized in that the hydrophobic radical is a radical according to formula X, Xa,

Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals comprising between 17 and 25 carbon atoms.

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals comprising between 17 and 18 carbon atoms.

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals represented by the formulas below:

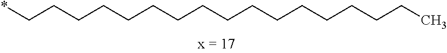 [SMILES: *CCCCCCCCCCCCCCCCC]

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals comprising between 19 and 25 carbon atoms.

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals represented by the formulas below:

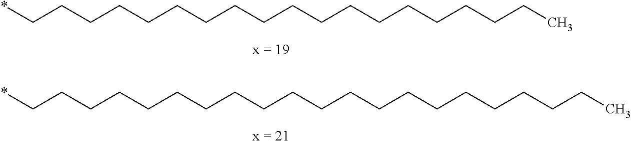 [SMILES: *CCCCCCCCCCCCCCCCCCC ; *CCCCCCCCCCCCCCCCCCCCC]

In one embodiment, the composition according to the invention is characterized in that the hydrophobic radical is a radical according to formula X, Xa, Xb, Xc, Xe, Xg, Xh, Xj, Xk, Xn wherein a′=1 or l′=1 wherein the radical GpC according to formula IX is chosen in the group consisting of radicals in which Cx is chosen in the group consisting of alkyl radicals comprising between 18 and 19 carbon atoms.

a′=2 or l′=2

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of linear alkyl radicals comprising between 9 and 15 carbon atoms.

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of branched alkyl radicals comprising between 9 and 15 carbon atoms.

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of alkyl radicals comprising 9 or 10 carbon atoms.

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of radicals represented by the formulas below:

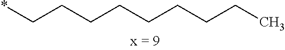 [SMILES: *CCCCCCCCC]

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of alkyl radicals comprising between 11 and 15 carbon atoms.

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of alkyl radicals comprising between 11 and 13 carbon atoms.

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of radicals represented by the formulas below:

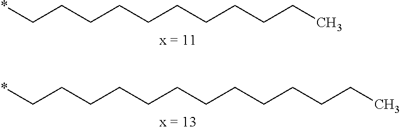 [SMILES: *CCCCCCCCCCC ; *CCCCCCCCCCCCC]

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of alkyl radicals comprising 14 or 15 carbon atoms.

In one embodiment, the composition according to the invention is characterized in that the hydrophobic radical is a radical according to formula X, Xa, Xb, Xc, Xf, Xg, Xi, Xj, Xl, Xp wherein a′=2 or l′=2 wherein the radical GpC according to formula IX is chosen in the group consisting of radicals in which Cx is chosen in the group consisting of radicals represented by the formulas below:

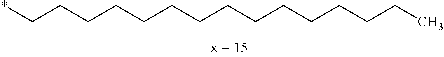 [SMILES: *CCCCCCCCCCCCCCC]

In one embodiment, the composition according to the invention is characterized in that the hydrophobic radical is a radical according to formula X, Xa, Xb, Xc, Xg, Xj, Xm wherein a′=3 wherein the radical GpC according to formula IX is chosen in the group consisting of radicals in which Cx is chosen in the group consisting of linear alkyl radicals comprising between 7 and 13 carbon atoms.

In one embodiment, the composition according to the invention is characterized in that the hydrophobic radical is a radical according to formula X, Xa, Xb, Xc, Xg, Xj, Xm wherein a′=3 wherein the radical GpC according to formula IX is chosen in the group consisting of radicals in which Cx is chosen in the group consisting of branched alkyl radicals comprising between 7 and 13 carbon atoms.

In one embodiment, the composition according to the invention is characterized in that the hydrophobic radical is a radical according to formula X, Xa, Xb, Xc, Xg, Xj, Xm wherein a′=3 wherein the radical GpC according to formula IX is chosen in the group consisting of radicals in which Cx is chosen in the group consisting of alkyl radicals comprising 7, 9 or 11 carbon atoms.

When the copolyamino acid comprises one or more aspartic unit(s), the latter may be subject to structural rearrangements.

In one embodiment, the composition according to the invention is characterized in that the copolyamino acid bearing carboxylate charges and at least a hydrophobic radical -Hy is chosen among copolyamino acids according to the following formula XXXa:

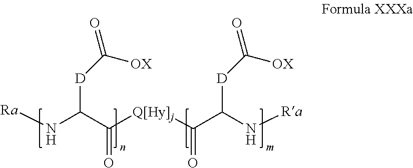

wherein,
D represents, independently, either a —CH₂— group (aspartic unit) or a —CH₂—CH₂— group (glutamic unit),
X represents a cationic entity chosen in the group comprising alkali metal cations,
Rₐ and Rₐ′, identical or different, are a radical chosen in the group consisting of a H, a C2 to C10 linear acyl group, a C3 to C10 branched acyl group, a benzyl, a terminal «amino acid» unit and a pyroglutamate,
Q, Hy and j are as defined above.
n+m represents the degree of polymerisation DP of the copolyamino acid, that is the mean number of monomeric unit in a copolyamino acid chain and 5≤n+m≤250;

In one embodiment, the composition according to the invention is characterized in that the copolyamino acid bearing carboxylate charges and at least a hydrophobic radical -Hy is chosen among copolyamino acids according to formula XXXa wherein Rₐ and Rₐ′, identical or different, are chosen in the group consisting of a H and a pyroglutamate.

In one embodiment, the composition according to the invention is characterized in that the copolyamino acid bearing carboxylate charges and at least a hydrophobic radical -Hy is chosen among copolyamino acids according to the following formula XXXa′:

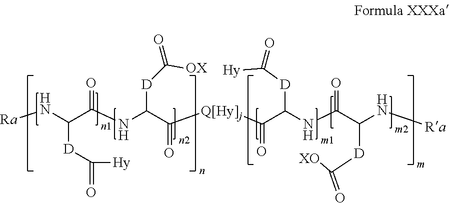

wherein:
D represents, independently, either a —CH2- group (aspartic unit) or a —CH2-CH2- group (glutamic unit),
X represents a cationic entity chosen in the group comprising alkali metal cations,
Q, Hy and j are as defined above.
Rₐ and Rₐ′, identical or different, are a radical chosen in the group consisting of a H, a C2 to C10 linear acyl group, a C3 to C10 branched acyl group, a benzyl, a terminal «amino acid» unit and a pyroglutamate,
n₁+m₁ represents the number of glutamic or aspartic units of the PLG chains of the copolyamino acid bearing a radical -Hy,
n₂+m₂ represents the number of glutamic or aspartic units of the PLG chains of the copolyamino acid not bearing a radical -Hy,
n₁+n₂=n and m₁+m₂=m
n+m represents the degree of polymerisation DP of the copolyamino acid, that is the mean number of monomeric unit in a copolyamino acid chain and 5≤n+m≤250;

In one embodiment, the composition according to the invention is characterized in that the copolyamino acid bearing carboxylate charges and at least a hydrophobic radical -Hy is chosen among copolyamino acids according to the following formula XXXa″:

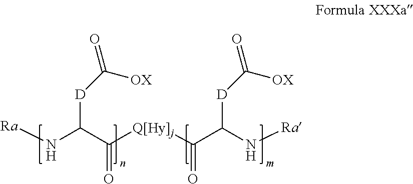

wherein:
D represents, independently, either a —CH2- group (aspartic unit) or a —CH2-CH2- group (glutamic unit),
X represents a cationic entity chosen in the group comprising alkali metal cations,
Q, Hy and j are as defined above.
Rₐ and Rₐ′, identical or different, are at least a hydrophobic radical -Hy and a radical chosen in the group consisting of -Hy, a H, a C2 to C10 linear acyl group, a C3 to C10 branched acyl group, a benzyl, a terminal «amino acid» unit and a pyroglutamate,
n+m represents the degree of polymerisation DP of the copolyamino acid, that is the mean number of monomeric unit in a copolyamino acid chain and 5≤n+m≤250;

In one embodiment, the composition according to the invention is characterized in that the copolyamino acid bearing carboxylate charges and at least a hydrophobic radical -Hy is chosen among copolyamino acids according to the following formula

### XXXb:

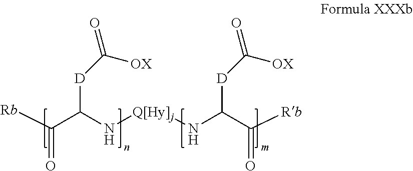

wherein,
D represents, independently, either a —CH2- group (aspartic unit) or a —CH2-CH2- group (glutamic unit),
R₂ represents a radical or spacer according to formula Q[-*]ᵢ as previously defined,
X represents a cationic entity chosen in the group comprising alkali metal cations,
R_{b} and R_{b}′, identical or different, are a —NR′R″ radical, R′ and R″ identical or different being chosen in the group consisting of H, C2 to C10 linear or branched or cyclic alkyls, the benzyl and said R′ and R″ alkyl may form together one or several saturated, instaurated and/or aromatic carbon rings and/or may comprise heteroatoms, chosen in the group consisting of O, N and S;
Q, Hy and j are as defined above.
n+m represents the degree of polymerisation DP of the copolyamino acid, that is the mean number of monomeric unit in a copolyamino acid chain and 5≤n+m≤250.

### XXXb′

In one embodiment, the composition according to the invention is characterized in that the copolyamino acid bearing carboxylate charges and at least a hydrophobic radical -Hy is chosen among copolyamino acids according to the following formula XXXb′:

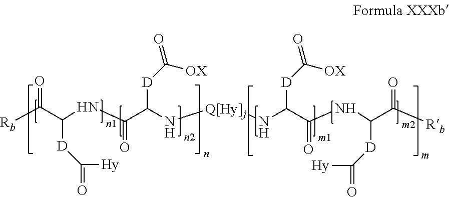

wherein:
D represents, independently, either a —CH2- group (aspartic unit) or a —CH2-CH2- group (glutamic unit),
X represents a cationic entity chosen in the group comprising alkali metal cations,
Q, Hy and j are as defined above.
R_{b} and R_{b}′, identical or different, are a —NR′R″ radical, R′ and R″ identical or different being chosen in the group consisting of H, C2 to C10 linear or branched or cyclic alkyls, the benzyl and said R′ and R″ alkyl may form together one or several saturated, instaurated and/or aromatic carbon rings and/or may comprise heteroatoms, chosen in the group consisting of O, N and S;
n1+m1 represents the number of glutamic or aspartic units of the PLG chains of the copolyamino acid bearing a radical -Hy
n2+m2 represents the number of glutamic or aspartic units of the PLG chains of the copolyamino acid not bearing a radical -Hy
n1+n2=n and m1+m2=m
   n+m represents the degree of polymerisation DP of the copolyamino acid, that is the mean number of monomeric unit in a copolyamino acid chain and 5≤n+m≤250;

In one embodiment, the composition according to the invention is characterized in that the copolyamino acid bearing carboxylate charges and at least a hydrophobic radical -Hy is chosen among copolyamino acids according to the following formula XXXb″:

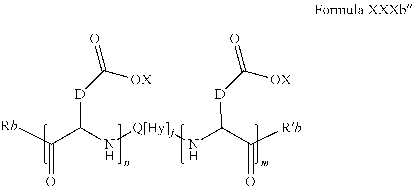

wherein:
D represents, independently, either a —CH2- group (aspartic unit) or a —CH2-CH2- group (glutamic unit),
X represents a cationic entity chosen in the group comprising alkali metal cations,
R_{b} and R_{b}′, identical or different, are at least a hydrophobic radical -Hy and a radical chosen in the group consisting of a hydrophobic radical -Hy and a —NR′R″ radical, R′ and R″ identical or different being chosen in the group consisting of H, C2 to C10 linear or branched or cyclic alkyls, the benzyl and said R′ and R″ alkyl may form together one or several saturated, instaurated and/or aromatic carbon rings and/or may comprise heteroatoms, chosen in the group consisting of O, N and S;
Q, Hy and j are as defined above.
n+m represents the degree of polymerisation DP of the copolyamino acid, that is the mean number of monomeric unit in a copolyamino acid chain and 5≤n+m≤250;

In one embodiment, the composition according to the invention is characterized in that when the co-polyamino acids comprises aspartate units, then the co-polyamino acids may further comprise monomeric units according to formula XXXX and/or XXXX′:

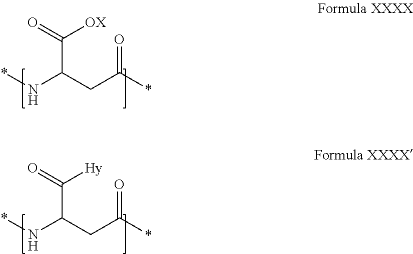

The term «random grafting copolyamino acid» refers to a copolyamino acid bearing carboxylate charges and at least a hydrophobic radical, that is a copolyamino acid according to formulas XXXa′ and XXXb′.

The term «defined grafting copolyamino acid» refers to a copolyamino acid bearing carboxylate charges and at least a hydrophobic radical, that is a copolyamino acid according to formulas XXXa, XXXa′″, XXXb and XXXb′″.

In one embodiment, the composition according to the invention is characterized in that the copolyamino acid bearing carboxylate charges and hydrophobic radicals is chosen among copolyamino acids according to formula XXXa, XXXa′, XXXa″, XXXb, XXXb′ or XXXb″ in which the copolyamino acid is chosen among copolyamino acids in which the group D is a —CH₂— group (unit aspartic).

In one embodiment, the composition according to the invention is characterized in that the copolyamino acid bearing carboxylate charges and hydrophobic radicals is chosen among copolyamino acids according to formula XXXa, XXXa′, XXXa″, XXXb, XXXb′ or XXXb″ in which the copolyamino acid is chosen among copolyamino acids in which the group D is a —CH₂—CH₂— group (unit glutamic).

The ratio of hydrophobic radical to basal insulin is defined as being the ratio of their respective molar concentrations: [Hy]/[basal insulin] (mol/mol) to obtain the expected performances, that is, the solubilization of basal insulin at a pH from 6.0 to 8.0, the precipitation of the basal insulin and the stability of the compositions according to the invention.

The minimum measured value of the ratio of hydrophobic radical to basal insulin [Hy]/[basal insulin], is the value at which the basal insulin is solubilized, because solubilization is the minimum effect to be obtained; this solubilization is a condition for all other technical effects which can only be observed if the basal insulin is solubilized at a pH from 6.0 to 8.0.

In the compositions according to the invention, the ratio of hydrophobic radical to basal insulin [Hy]/[basal insulin] may be greater than the minimum value determined by the solubilization limit.

In one embodiment, the ratio of hydrophobic radical by basal insulin [Hy]/[basal insulin]≤2.

In one embodiment, the ratio of hydrophobic radical by basal insulin [Hy]/[basal insulin]≤1.75.

In one embodiment, the ratio of hydrophobic radical by basal insulin [Hy]/[basal insulin]≤1.5.

In one embodiment, the ratio of hydrophobic radical by basal insulin [Hy]/[basal insulin]≤1.25.

In one embodiment, the ratio of hydrophobic radical by basal insulin [Hy]/[basal insulin]≤1.00.

In one embodiment, the ratio of hydrophobic radical by basal insulin [Hy]/[basal insulin]≤0.75.

In one embodiment, the ratio of hydrophobic radical by basal insulin [Hy]/[basal insulin]≤0.5.

In one embodiment, the ratio of hydrophobic radical by basal insulin [Hy]/[basal insulin]≤0.25.

In one embodiment, the composition according to the invention is characterized in that the ratio M between the number of hydrophobic radicals and the number of glutamic or aspartic units is comprised from 0.007 and 0.3.

In one embodiment, the composition according to the invention is characterized in that the ratio M between the number of hydrophobic radicals and the number of glutamic or aspartic units is comprised from 0.01 and 0.3.

In one embodiment, the composition according to the invention is characterized in that the ratio M between the number of hydrophobic radicals and the number of glutamic or aspartic units is comprised from 0.02 and 0.2.

In one embodiment, the composition according to the invention is characterized in that n+m is comprised from 10 and 200.

In one embodiment, the composition according to the invention is characterized in that n+m is comprised from 15 and 150.

In one embodiment, the composition according to the invention is characterized in that n+m is comprised from 15 and 100.

In one embodiment, the composition according to the invention is characterized in that n+m is comprised from 15 and 80.

In one embodiment, the composition according to the invention is characterized in that n+m is comprised from 15 and 65.

In one embodiment, the composition according to the invention is characterized in that n+m is comprised from 20 and 60.

In one embodiment, the composition according to the invention is characterized in that n+m is comprised from 20 and 50.

In one embodiment, the composition according to the invention is characterized in that n+m is comprised from 20 and 40.

The invention further relates to a preparation method of stable injectable compositions.

In one embodiment, the invention also relates to the precursors of said hydrophobic radicals according to formula X.

In one embodiment, the invention also relates to the copolyamino acid according to formula I

Q[HY]ⱼ[PLG]ₖ Formula I

wherein:

j≥1; k≥2
said copolyamino acid according to formula I bearing carboxylate charges and consisting of at least two chains of PLG glutamic or aspartic units bound together by a linear or branched radical or spacer Q[-*]i (i≥3 with i=j+k) at least trivalent consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl groups said radical Q[-*]ᵢ bearing at least a monovalent hydrophobic radical -Hy;
said radical or spacer Q[-*]ᵢ being bound to at least two chains of PLG glutamic or aspartic units by an amide function and,
said radical or spacer Q[-*]ᵢ being bound to at least a hydrophobic radical -Hy according to formula X hereafter defined by an amide function.
Said amide function binding said radical or spacer Q[-*]ᵢ to at least two chains of glutamic or aspartic units comes from the reaction between an amine function and an acid function respectively carried by either the precursor Q′ of the radical or spacer Q[-*]ᵢ or a glutamic or aspartic unit.
the amide function binding said radical or spacer Q[-*]ᵢ to, at least a hydrophobic radical -Hy according to formula X comes from the reaction between an amine function and an acid function respectively carried by either the precursor Q′ of the radical or spacer Q[-*]ᵢ or the precursor Hy′ of the hydrophobic radical -Hy,
The radical -Hy being defined above,
The radical or spacer Q[-*]ᵢ being defined above.

In one embodiment, the invention also relates to the compound according to formula Ib which is the precursor of the copolyamino acid according to formula I defined above:

Q′″[Hy]ⱼ Formula Ib

wherein:

j≥1
said compound according to formula Ib being constitued of the precursor of the linear or branched radical or spacer Q′″[-*]j consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl groups said radical Q′″[-*]ⱼ bearing at least a monovalent hydrophobic radical -Hy bound by amide bonds, and
the precursor of the radical or spacer Q′″[-*]ⱼ bearing at least k free amine or acid reactive functions,
Said amide functions binding said radical or spacer Q′″[-*]ⱼ to the at least hydrophobic radical -Hy from the reaction between an amine function and an acid function respectively carried by either the precursor Q′ of the radical or spacer Q′″[-*]ⱼ or the precursor Hy′ of the hydrophobic radical -Hy,
Said radical or spacer Q′″[-*]₋ⱼ being chosen among radicals according to formula Q[-*]ⱼ=([Q′]_{q})[-*]ⱼ, wherein 1≤q≤5
   radicals Q′ being identical or different and chosen in the group consisting of radicals according to formula III to VI defined above, to form Q[-*]ⱼ (j≥3), which identical or different functions Fx=Fa, Fb, Fc, Fd, Fa′, Fb′, Fc′, Fc″ and Fd′ that represent functions —NH— or —CO— and Fy represents a trivalent nitrogen atom —N═,
   two radicals Q′ being bound between them by a covalent bond and a carboxyl function, Fx=—CO—, and an amine function Fx=—NH— or Fy=—N═, thus forming an amide bond,
   and when the at least two reactive functions are not bound to a radical Q′ or to a hydrophobic radical -Hy they constitute free carboxylic or amine functions.

In one embodiment, the invention also relates to the copolyamino acid according to formula Ia which is the precursor of the copolyamino acid according to formula I defined above:

Q″[PLG]ₖ Formula Ia

wherein:

k≥2
said copolyamino acid according to formula Ia bearing carboxylate charges and consisting of at least two chains of PLG glutamic or aspartic units bound between them by the precursor of the linear or branched radical or spacer Q″[-*]k consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl groups,
said radical or spacer Q″[-*]ₖ being bound to the at least two chains of PLG glutamic or aspartic units by an amide function and, bearing after binding to the at least two chains of PLG glutamic or aspartic units at least j reactive free amine or acid functions
Said amide functions binding said radical or spacer Q″[-*]ₖ to at least two chains of glutamic or aspartic units come from the reaction between an amine function and an acid function respectively carried by either the precursor Q′ of the radical or spacer Q″[-*]k or a glutamic or aspartic unit,
Said radical or spacer Q″[-*]ₖ being chosen among radicals according to formula Q″[-*]k=([Q′]_{q})[-*]ₖ, wherein 1≤q≤5
   radicals Q′ being identical or different and chosen in the group consisting of radicals according to formula III to VI defined above, to form Q[-*]ₖ (k≥3), which function Fx=Fa, Fb, Fc, Fd, Fa′, Fb′, Fc′, Fc″ and Fd′ identical or different represent functions —NH— or —CO— and Fy represents a trivalent nitrogen atom —N═,
   two radicals Q′ being bound between them by a covalent bond between a carboxyl function, Fx=—CO—, and an amine function Fx=—NH— or Fy=—N═, thus forming an amide bond,
   and when the at least reactive function is not bound to a radical Q′ or to at least two chains of glutamic or aspartic units they constitute free carboxylic acid or amine functions.

In one embodiment, the composition according to the invention is characterized in that the PLG chains that form the copolyamino acid are obtained by polymerisation.

In one embodiment, the composition according to the invention is characterized in that the PLG chains that form the copolyamino acid are obtained by polymerisation by ring-opening of a N-carboxyanhydride glutamic acid derivative or a N-carboxyanhydride aspartic acid derivative.

In one embodiment, the composition according to the invention is characterized in that the PLG chains that form the copolyamino acid are obtained by polymerisation of a N-carboxyanhydride glutamic acid derivative or a N-carboxyanhydride aspartic acid derivative as described in the article Adv. Polym. Sci. 2006, 202, 1-18 (Deming, T. J.).

In one embodiment, the composition according to the invention is characterized in that the PLG chains that form the copolyamino acid are obtained by polymerisation of a N-carboxyanhydride glutamic acid derivative.

In one embodiment, the composition according to the invention is characterized in that the PLG chains that form the copolyamino acid are obtained by polymerisation of a N-carboxyanhydride glutamic acid derivative chosen in the group constituted by the N-carboxyanhydride methyl polyglutamate (GluOMe-NCA), the N-carboxyanhydride benzyl polyglutamate (GluOBzl-NCA) and the N-carboxyanhydride tert-butyle polyglutamate (GluOtBu-NCA).

In one embodiment, the N-carboxyanhydride glutamic acid derivative is the N-carboxyanhydride methyl poly-L-glutamate (L-GluOMe-NCA).

In one embodiment, the N-carboxyanhydride glutamic acid derivative is the N-carboxyanhydride benzyl poly-L-glutamate (L-GluOBzl-NCA).

In one embodiment, the composition according to the invention is characterized in that the PLG chains that form the copolyamino acid are obtained by polymerisation of a N-carboxyanhydride glutamic acid derivative or a N-carboxyanhydride aspartic acid derivative using a transition metal organometallic complexe as initiator as described in Nature 1997, 390, 386-389 (Deming, T. J.).

In one embodiment, the composition according to the invention is characterized in that the PLG chains that form the copolyamino acid are obtained by polymerisation of a N-carboxyanhydride glutamic acid derivative or a N-carboxyanhydride aspartic acid derivative using ammonia or a primary amine as initiator as described in patent FR 2,801,226 (Torraud, F.; and al.) and the references cited in this patent.

In one embodiment, the composition according to the invention is characterized in that the PLG chains that form the copolyamino acid are obtained by polymerisation of a N-carboxyanhydride glutamic acid derivative or a N-carboxyanhydride aspartic acid derivative using the hexamethyldisilazane as initiator as described in the article J. Am. Chem. Soc. 2007, 129, 14114-14115 (Lu H.; and al.) or a silylated amine as described in the article J. Am. Chem. Soc. 2008, 130, 12562-12563 (Lu H.; and al.).

In one embodiment, the composition according to the invention is characterized in that the PLG chains that form the copolyamino acid are obtained by polymerisation of a N-carboxyanhydride glutamic acid derivative or of a N-carboxyanhydride aspartic acid derivative, the polymerisation is initiated by the amine functions carried by the radical or spacer Q[-*]ᵢ.

In one embodiment, the composition according to the invention is characterized in that the PLG chains that form the copolyamino acid are obtained by polymerisation of a N-carboxyanhydride glutamic acid derivative chosen in the group consisting of the N-carboxyanhydride methyl polyglutamate (GluOMe-NCA), the N-carboxyanhydride benzyl polyglutamate (GluOBzl-NCA) and the N-carboxyanhydride tert-butyl polyglutamate (GluOtBu-NCA), the polymerisation is initiated by the amine functions carried by the radical or spacer Q[-*]ᵢ.

In one embodiment, the composition according to the invention is characterized in that the PLG chains that form the copolyamino acid are obtained by polymerisation of the N-carboxyanhydride methyl poly-L-glutamate (L-GluOMe-NCA), the polymerisation is initiated by the amine functions carried by the radical or spacer Q[-*]ᵢ.

In one embodiment, the composition according to the invention is characterized in that the PLG chains that form the copolyamino acid are obtained by polymerisation of the N-carboxyanhydride benzyl poly-L-glutamate (L-GluOBzl-NCA), the polymerisation is initiated by the amine functions carried by the radical or spacer Q[-*]ᵢ.

In one embodiment, the composition according to the invention is characterized in that the PLG chains that form the copolyamino acid are obtained by polymerisation of a N-carboxyanhydride glutamic acid derivative or of a N-carboxyanhydride aspartic acid derivative, the polymerisation is initiated by the amine functions carried by the precursor of the radical Q[-*]ₖ[Hy]ⱼ.

In one embodiment, the composition according to the invention is characterized in that the PLG chains that form the copolyamino acid are obtained by polymerisation of a N-carboxyanhydride glutamic acid derivative chosen in the group consisting of the N-carboxyanhydride methyl polyglutamate (GluOMe-NCA), the N-carboxyanhydride benzyl polyglutamate (GluOBzl-NCA) and the N-carboxyanhydride tert-butyl polyglutamate (GluOtBu-NCA), the polymerisation is initiated by the amine functions carried by the precursor of the radical Q[-*]ₖ[Hy]ⱼ.

In one embodiment, the composition according to the invention is characterized in that the PLG chains that form the copolyamino acid are obtained by polymerisation of the N-carboxyanhydride methyl poly-L-glutamate (L-GluOMe-NCA), the polymerisation is initiated by the amine functions carried by the precursor of the radical Q[-*]ₖ[Hy]ⱼ.

In one embodiment, the composition according to the invention is characterized in that the PLG chains that form the copolyamino acid are obtained by polymerisation of the N-carboxyanhydride benzyl poly-L-glutamate (L-GluOBzl-NCA), the polymerisation is initiated by the amine functions carried by the precursor of the radical Q[-*]ₖ[Hy]ⱼ.

In one embodiment, the composition according to the invention is characterized in that the process for synthesizing the polyaminoacid obtained by polymerization of a N-carboxyanhydride glutamic acid derivative or a N-carboxyanhydride aspartic acid derivative from which the copolyamino acid is obtained comprises a step of ester function hydrolysis.

In one embodiment, this ester function hydrolysis step may consist of hydrolysis in an acidic medium or hydrolysis in a basic medium or may be carried out by hydrogenation.

In one embodiment, this ester group hydrolysis step is a hydrolysis in an acidic medium.

In one embodiment, this ester group hydrolysis step is carried out by hydrogenation.

In one embodiment, the composition according to the invention is characterized in that the PLG chains that form the copolyamino acid are obtained from a polyamino acid obtained via depolymerization of a polyamino acid of higher molecular weight.

In one embodiment, the composition according to the invention is characterized in that the PLG chains that form the copolyamino acid are obtained from a polyamino acid obtained via enzymatic depolymerization of a polyamino acid of higher molecular weight.

In one embodiment, the composition according to the invention is characterized in that the PLG chains that form the copolyamino acid are obtained from a polyamino acid obtained via chemical depolymerization of a polyamino acid of higher molecular weight.

In one embodiment, the composition according to the invention is characterized in that the PLG chains constituent the copolyamino acid are obtained from a polyamino acid obtained via enzymatic and chemical depolymerization of a polyamino acid of higher molecular weight.

In one embodiment, the composition according to the invention is characterized in that the PLG chains that form the copolyamino acid are obtained from a polyamino acid obtained via depolymerization of a polyamino acid of higher molecular weight chosen in the group consisting of the sodium polyglutamate and the sodium polyaspartate.

In one embodiment, the composition according to the invention is characterized in that the PLG chains that form the copolyamino acid are obtained from a polyamino acid obtenu by depolymerisation of a sodium polyglutamate of higher molecular weight.

In one embodiment, the composition according to the invention is characterized in that the PLG chains that form the copolyamino acid are obtained from a polyamino acid obtenu by depolymerisation of a sodium polyaspartate of higher molecular weight.

In one embodiment, the composition according to the invention is characterized in that the amide bonds in the copolyamino acid are obtained from a process to form amide bonds well known to those skilled in the art.

In one embodiment, the composition according to the invention is characterized in that the amide bonds in the copolyamino acid are obtained from a process to form amide bonds used in peptide synthesis.

In one embodiment, the composition according to the invention is characterized in that the amide bonds in the copolyamino acid are obtained from a process to form amide bonds described in patent FR 2.840.614 (Chan, Y. P.; and al.).

In one embodiment, the composition according to the invention is characterized in that the amide bonds in the copolyamino acid between the PLG chains and the radical or spacer Q[-*]ᵢ and between the radical or spacer Q[-*]ᵢ and the hydrophobic radical -Hy are obtained from a process to form amide bonds well known to those skilled in the art.

In one embodiment, the composition according to the invention is characterized in that the amide bonds in the copolyamino acid between the PLG chains and the radical or spacer Q[-*]ᵢ and between the radical or spacer Q[-*]ᵢ and the hydrophobic radical -Hy are obtained from a process to form amide bonds used in peptide synthesis.

In one embodiment, the composition according to the invention is characterized in that the amide bonds in the copolyamino acid between the PLG chains and the radical or spacer Q[-*]ᵢ and between the radical or spacer Q[-*]ᵢ and the hydrophobic radical -Hy are obtained from a process to form amide bonds described in patent FR 2.840.614 (Chan, Y. P.; and al.).

Hereinafter, the units used for insulins are those recommended by pharmacopoeias, whose mg/ml equivalences are provided in the table hereafter:

| | Pharmacopee EP 8.0 | Pharmacopee US - USP38 |
|---|---|---|
| Insulin | (2014) | (2015) |
| Aspart | 1 U = 0.0350 mg of insulin | 1 USP = 0.0350 mg of insulin |
| | aspart | aspart |
| Lispro | 1 U = 0.0347 mg d'insulin | 1 USP = 0.0347 mg d'insulin |
| | lispro | lispro |
| Humaine | 1 UI = 0.0347 mg of | 1 USP = 0.0347 mg of insulin |
| | insulin humaine | humaine |
| Glargine | 1 U = 0.0364 mg d'insulin | 1 USP = 0.0364 mg d'insulin |
| | glargine | glargine |
| Porcine | 1 UI = 0.0345 mg of | 1 USP = 0.0345 mg of insulin |
| | insulin porcine | porcine |
| Bovine | 1 UI = 0.0342 mg of insulin | 1 USP = 0.0342 mg of insulin |
| | bovine | bovine |

By basal insulin with an isoelectric point from 5.8 to 8.5 is meant an insoluble insulin at pH 7 and whose duration of action is comprised from 8 to 24 hours or longer in standard diabetes models.

These basal insulins, whose isoelectric point is comprised from 5.8 to 8.5, are recombinant insulins whose primary structure has been modified mainly by introducing basic amino acids such as Arginine or Lysine. They are described, for example, in the following patents, patent applications or publications: WO 2003/053339, WO 2004/096854, U.S. Pat. Nos. 5,656,722 and 6,100,376, the content of which is incorporated by reference.

In one embodiment, the basal insulin with an isoelectric point from 5.8 to 8.5 is insulin glargine. Insulin glargine is marketed under the brand Lantus® (100 U/ml) or Toujeo® (300 U/ml) by SANOFI.

In one embodiment, the basal insulin with an isoelectric point from 5.8 to 8.5 is a bio-similar insulin glargine.

Bio-similar insulin glargine is being marketed under the brand Abasaglar® or Basaglar® by ELI LILLY.

In one embodiment, the compositions according to the invention comprise from 40 to 500 U/mL of basal insulin for which the isoelectric point is comprised from 5.8 to 8.5.

In one embodiment, compositions according to the invention comprise 40 U/mL of basal insulin which isoelectric point is comprised from 5.8 to 8.5.

In one embodiment, compositions according to the invention comprise 100 U/mL (that is about 3.6 mg/mL) of basal insulin which isoelectric point is comprised from 5.8 to 8.5.

In one embodiment, compositions according to the invention comprise 150 U/mL of basal insulin which isoelectric point is comprised from 5.8 to 8.5.

In one embodiment, compositions according to the invention comprise 200 U/mL of basal insulin which isoelectric point is comprised from 5.8 to 8.5.

In one embodiment, compositions according to the invention comprise 225 U/mL of basal insulin which isoelectric point is comprised from 5.8 to 8.5.

In one embodiment, compositions according to the invention comprise 250 U/mL of basal insulin which isoelectric point is comprised from 5.8 to 8.5.

In one embodiment, compositions according to the invention comprise 300 U/mL of basal insulin which isoelectric point is comprised from 5.8 to 8.5.

In one embodiment, compositions according to the invention comprise 400 U/mL of basal insulin which isoelectric point is comprised from 5.8 to 8.5.

In one embodiment, compositions according to the invention comprise 500 U/mL of basal insulin which isoelectric point is comprised from 5.8 to 8.5.

In one embodiment, the mass ratio between the basal insulin, which isoelectric point is comprised from 5.8 to 8.5, and the copolyamino acid, or copolyamino acid/basal insulin, is comprised from 0.2 and 8.

In one embodiment, the mass ratio is comprised from 0.2 and 6.

In one embodiment, the mass ratio is comprised from 0.2 and 5.

In one embodiment, the mass ratio is comprised from 0.2 and 4.

In one embodiment, the mass ratio is comprised from 0.2 and 3.

In one embodiment, the mass ratio is comprised from 0.2 and 2.

In one embodiment, the mass ratio is comprised from 0.2 and 1.

In one embodiment, the concentration in copolyamino acid bearing carboxylate charges and hydrophobic radicals is at most of 60 mg/mL.

In one embodiment, the concentration in copolyamino acid bearing carboxylate charges and hydrophobic radicals is at most of 40 mg/mL.

In one embodiment, the concentration in copolyamino acid bearing carboxylate charges and hydrophobic radicals is at most of 20 mg/mL.

In one embodiment, the concentration in copolyamino acid bearing carboxylate charges and hydrophobic radicals is at most of 10 mg/mL.

In one embodiment, the concentration in copolyamino acid bearing carboxylate charges and hydrophobic radicals is at most of 5 mg/ml.

In one embodiment, the concentration in copolyamino acid bearing carboxylate charges and hydrophobic radicals is at most of 2.5 mg/ml.

In one embodiment, compositions according to the invention further comprise a prandial insulin. Prandial insulins are soluble at pH 7.

Prandial insulin means a so-called rapid or “regular” insulin.

So-called rapid prandial insulins are insulins which must respond to the needs caused by the ingestion of proteins and sugars during a meal; they must act in less than 30 minutes.

In one embodiment, the so-called “regular” prandial insulin is human insulin.

In one embodiment, the prandial insulin is a recombinant human insulin as described in European Pharmacopeia and American Pharmacopeia.

Human insulin is marketed, for example, under the brands Humulin® (ELI LILLY) and Novolin® (NOVO NORDISK).

So-called fast acting prandial insulins are insulins which are obtained by recombination and whose primary structure has been modified to decrease their acting time.

In one embodiment, so-called fast acting prandial insulins are chosen from the group comprising insulin lispro (Humaloe), insulin glulisine (Apidra®) and insulin aspart (NovoLoe).

In one embodiment, the prandial insulin is insulin lispro.

In one embodiment, the prandial insulin is insulin glulisine.

In one embodiment, the prandial insulin is insulin aspart.

In one embodiment, compositions according to the invention comprise in total between 40 and 500 U/mL of insulin with a combination of prandial insulin and basal insulin which isoelectric point is comprised from 5.8 to 8.5.

In one embodiment, compositions according to the invention comprise in total between 60 and 800 U/mL of insulin with a combination of prandial insulin and basal insulin which isoelectric point is comprised from 5.8 to 8.5.

In one embodiment, compositions according to the invention comprise in total between 100 and 500 U/mL of insulin with a combination of prandial insulin and basal insulin which isoelectric point is comprised from 5.8 to 8.5.

In one embodiment, compositions according to the invention comprise in total 800 U/mL of insulin with a combination of prandial insulin and basal insulin which isoelectric point is comprised from 5.8 to 8.5.

In one embodiment, compositions according to the invention comprise in total 700 U/mL of insulin with a combination of prandial insulin and basal insulin which isoelectric point is comprised from 5.8 to 8.5.

In one embodiment, compositions according to the invention comprise in total 600 U/mL of insulin with a combination of prandial insulin and basal insulin which isoelectric point is comprised from 5.8 to 8.5.

In one embodiment, compositions according to the invention comprise in total 500 U/mL of insulin with a combination of prandial insulin and basal insulin which isoelectric point is comprised from 5.8 to 8.5.

In one embodiment, compositions according to the invention comprise in total 400 U/mL of insulin with a combination of prandial insulin and basal insulin which isoelectric point is comprised from 5.8 to 8.5.

In one embodiment, compositions according to the invention comprise in total 300 U/mL of insulin with a combination of prandial insulin and basal insulin which isoelectric point is comprised from 5.8 to 8.5.

In one embodiment, compositions according to the invention comprise in total 266 U/mL of insulin with a combination of prandial insulin and basal insulin which isoelectric point is comprised from 5.8 to 8.5.

In one embodiment, compositions according to the invention comprise in total 200 U/mL of insulin with a combination of prandial insulin and basal insulin which isoelectric point is comprised from 5.8 to 8.5.

In one embodiment, compositions according to the invention comprise in total 100 U/mL of insulin with a combination of prandial insulin and basal insulin which isoelectric point is comprised from 5.8 to 8.5.

The proportions between the basal insulin for which the isoelectric point is comprised from 5.8 to 8.5 and the prandial insulin are for example in percentage of from 25/75, 30/70, 40/60, 50/50, 60/40, 63/37, 70/30, 75/25, 80/20, 83/17, 90/10 for formulations as described above consisting from 60 to 800 U/mL. However, any other proportion can be achieved.

In one embodiment, compositions according to the invention comprising in total 40 U/mL of insulin with a combination of prandial insulin and basal insulin which isoelectric point is comprised from 5.8 to 8.5.

In one embodiment, compositions according to the invention further comprise a gastrointestinal hormone.

By “gastrointestinal hormones” is meant hormones chosen from the group consisting of GLP-1 RA (Glucagon-like peptide-1 receptor agonist) and GIP (Glucose-dependent insulinotropic peptide), oxyntomodulin (a derivative of proglucagon), YY peptide, amylin, cholecystokinin, pancreatic peptide (PP), ghrelin and enterostatin, their analogues or derivatives and/or their pharmaceutically acceptable salts.

In one embodiment, the gastrointestinal hormones are analogues or derivatives of GLP-1 RA chosen from the group consisting of exenatide or Byetta® (ASTRA-ZENECA), liraglutide or Victoza® (NOVO NORDISK), lixisenatide or Lyxumia® (SANOFI), albiglutide or Tanzeum® (GSK) or dulaglutide or Trulicity® (ELI LILLY & CO), their analogues or derivatives and/or their pharmaceutically acceptable salts.

In one embodiment, the gastrointestinal hormone is pramlintide or Symlin® (ASTRA-ZENECA).

In one embodiment, the gastrointestinal hormone is exenatide or Byetta®, its analogues or derivatives and their pharmaceutically acceptable salts.

In one embodiment, the gastrointestinal hormone is liraglutide or Victoza®, its analogues or derivatives and their pharmaceutically acceptable salts.

In one embodiment, the gastrointestinal hormone is lixisenatide or Lyxumia®, its analogues or derivatives and their pharmaceutically acceptable salts.

In one embodiment, the gastrointestinal hormone is albiglutide or Tanzeum®, its analogues or derivatives and their pharmaceutically acceptable salts.

In one embodiment, the gastrointestinal hormone is dulaglutide or Trulicity®, its analogues or derivatives and their pharmaceutically acceptable salts.

In one embodiment, the gastrointestinal hormone is pramlintide or Symlin®, its analogues or derivatives and their pharmaceutically acceptable salts.

The term “analogue” means, when used with reference to a peptide or a protein, a peptide or a protein, which one or a plurality of its constituent amino acid residues have been substituted by other amino acid residues and/or which one or a plurality of its constituent amino acid residues have been removed and/or which one or a plurality of its constituent amino acid residues have been added. The percentage of homology allowed for the present definition of an analog is 50%.

The term “derivative” means, when used with reference to a peptide or a protein, a peptide or a protein or an analog chemically modified by a substituent which is not present in the reference peptide or protein or analog, i.e. A peptide or a protein which has been modified by creating covalent bonds, to introduce substituents.

In one embodiment, the substituent is chosen from the group consisting of fatty chains.

In one embodiment, the concentration of gastrointestinal hormone is comprised within a range from 0.01 to 100 mg/mL.

In one embodiment, the concentration of gastrointestinal hormone is comprised within a range from 0.01 to 10 mg/mL.

In one embodiment, the concentration of exenatide, its analogues or derivatives and their pharmaceutical acceptable salts is comprised within a range from 0.04 to 0.5 mg/mL.

In one embodiment, the concentration of liraglutide, its analogues or derivatives and their pharmaceutical acceptable salts is comprised within a range from 1 to 10 mg/mL.

In one embodiment, the concentration of lixisenatide, its analogues or derivatives and their pharmaceutical acceptable salts is comprised within a range from 0.01 to 1 mg/mL.

In one embodiment, the concentration of albiglutide, its analogues or derivatives and their pharmaceutical acceptable salts is comprised from 5 to 100 mg/mL.

In one embodiment, the concentration of dulaglutide, its analogues or derivatives and their pharmaceutical acceptable salts is comprised from 0.1 to 10 mg/mL.

In one embodiment, the concentration of pramlintide, its analogues or derivatives and their pharmaceutical acceptable salts is comprised from 0.1 to 5 mg/mL.

In one embodiment, the compositions according to the invention are obtained by mixing commercial solutions of basal insulin which isoelectric point is from 5.8 to 8.5 and commercial solutions of GLP-1 RA, analogue or derivative of GLP-1 RA in volume ratios within a range from 10/90 to 90/10.

In one embodiment, the composition according to the invention comprises a daily dose of basal insulin and a daily dose of gastrointestinal hormone.

In one embodiment, compositions according to the invention comprise between 40 U/mL and 500 U/mL of basal insulin which isoelectric point is comprised from 5.8 to 8.5 and, between 0.05 and 0.5 mg/mL of exenatide.

In one embodiment, compositions according to the invention comprise between 40 U/mL and 500 U/mL of basal insulin which isoelectric point is comprised from 5.8 to 8.5 and, from 1 to 10 mg/mL of liraglutide.

In one embodiment, compositions according to the invention comprise between 40 U/mL and 500 U/mL of basal insulin which isoelectric point is comprised from 5.8 to 8.5 and, from 0.01 to 1 mg/mL of lixisenatide.

In one embodiment, compositions according to the invention comprise between 40 U/mL and 500 U/mL of basal insulin which isoelectric point is comprised from 5.8 to 8.5 and, from 5 to 100 mg/mL of albiglutide.

In one embodiment, compositions according to the invention comprise between 40 U/mL and 500 U/mL of basal insulin which isoelectric point is comprised from 5.8 to 8.5 and, from 0.1 to 10 mg/mL of dulaglutide.

In one embodiment, compositions according to the invention comprise 500 U/mL of basal insulin which isoelectric point is comprised from 5.8 to 8.5 and, from 0.04 to 0.5 mg/mL of exenatide.

In one embodiment, compositions according to the invention comprise 500 U/mL of basal insulin which isoelectric point is comprised from 5.8 to 8.5 and, from 1 to 10 mg/mL of liraglutide.

In one embodiment, compositions according to the invention comprise 500 U/mL of basal insulin which isoelectric point is comprised from 5.8 to 8.5 and, from 0.01 to 1 mg/mL of lixisenatide.

In one embodiment, compositions according to the invention comprise 500 U/mL of basal insulin which isoelectric point is comprised from 5.8 to 8.5 and, from 5 to 100 mg/mL of albiglutide.

In one embodiment, compositions according to the invention comprise 500 U/mL of basal insulin which isoelectric point is comprised from 5.8 to 8.5 and, from 0.1 to 10 mg/mL of dulaglutide.

In one embodiment, compositions according to the invention comprise 400 U/mL of basal insulin which isoelectric point is comprised from 5.8 to 8.5 and, from 0.04 to 0.5 mg/mL of exenatide.

In one embodiment, compositions according to the invention comprise 400 U/mL of basal insulin which isoelectric point is comprised from 5.8 to 8.5 and, from 1 to 10 mg/mL of liraglutide.

In one embodiment, compositions according to the invention comprise 400 U/mL of basal insulin which isoelectric point is comprised from 5.8 to 8.5 and, from 0.01 to 1 mg/mL of lixisenatide.

In one embodiment, compositions according to the invention comprise 400 U/mL of basal insulin which isoelectric point is comprised from 5.8 to 8.5 and, from 5 to 100 mg/mL of albiglutide.

In one embodiment, compositions according to the invention comprise 400 U/mL of basal insulin which isoelectric point is comprised from 5.8 to 8.5 and, from 0.1 to 10 mg/mL of dulaglutide.

In one embodiment, compositions according to the invention comprise 300 U/mL of basal insulin which isoelectric point is comprised from 5.8 to 8.5 and, from 0.04 to 0.5 mg/mL of exenatide.

In one embodiment, compositions according to the invention comprise 300 U/mL of basal insulin which isoelectric point is comprised from 5.8 to 8.5 and, from 1 to 10 mg/mL of liraglutide.

In one embodiment, compositions according to the invention comprise 300 U/mL of basal insulin which isoelectric point is comprised from 5.8 to 8.5 and, from 0.01 to 1 mg/mL of lixisenatide.

In one embodiment, compositions according to the invention comprise 300 U/mL of basal insulin which isoelectric point is comprised from 5.8 to 8.5 and, from 5 to 100 mg/mL of albiglutide.

In one embodiment, compositions according to the invention comprise 300 U/mL of basal insulin which isoelectric point is comprised from 5.8 to 8.5 and, from 0.1 to 10 mg/mL of dulaglutide.

In one embodiment, compositions according to the invention comprise 225 U/mL of basal insulin which isoelectric point is comprised from 5.8 to 8.5 and, from 0.04 to 0.5 mg/mL of exenatide.

In one embodiment, compositions according to the invention comprise 225 U/mL of basal insulin which isoelectric point is comprised from 5.8 to 8.5 and, from 1 to 10 mg/mL of liraglutide.

In one embodiment, compositions according to the invention comprise 225 U/mL of basal insulin which isoelectric point is comprised from 5.8 to 8.5 and, from 0.01 to 1 mg/mL of lixisenatide.

In one embodiment, compositions according to the invention comprise 225 U/mL of basal insulin which isoelectric point is comprised from 5.8 to 8.5 and, from 5 to 100 mg/mL of albiglutide.

In one embodiment, compositions according to the invention comprise 225 U/mL of basal insulin which isoelectric point is comprised from 5.8 to 8.5 and, from 0.1 to 10 mg/mL of dulaglutide.

In one embodiment, compositions according to the invention comprise 200 U/mL of basal insulin which isoelectric point is comprised from 5.8 to 8.5 and, from 0.04 to 0.5 mg/mL of exenatide.

In one embodiment, compositions according to the invention comprise 200 U/mL of basal insulin which isoelectric point is comprised from 5.8 to 8.5 and, from 1 to 10 mg/mL of liraglutide.

In one embodiment, compositions according to the invention comprise 200 U/mL of basal insulin which isoelectric point is comprised from 5.8 to 8.5 and, from 0.01 to 1 mg/mL of lixisenatide.

In one embodiment, compositions according to the invention comprise 200 U/mL of basal insulin which isoelectric point is comprised from 5.8 to 8.5 and, from 5 to 100 mg/mL of albiglutide.

In one embodiment, compositions according to the invention comprise 200 U/mL of basal insulin which isoelectric point is comprised from 5.8 to 8.5 and, from 0.1 to 10 mg/mL of dulaglutide.

In one embodiment, compositions according to the invention comprise 100 U/mL (that is about 3.6 mg/mL) of basal insulin which isoelectric point is comprised from 5.8 to 8.5 and, from 0.04 to 0.5 mg/mL of exenatide.

In one embodiment, compositions according to the invention comprise 100 U/mL (that is about 3.6 mg/mL) of basal insulin which isoelectric point is comprised from 5.8 to 8.5 and, from 1 to 10 mg/mL of liraglutide.

In one embodiment, compositions according to the invention comprise 100 U/mL (that is about 3.6 mg/mL) of basal insulin which isoelectric point is comprised from 5.8 to 8.5 and, from 0.01 to 1 mg/mL of lixisenatide.

In one embodiment, compositions according to the invention comprise 100 U/mL of basal insulin which isoelectric point is comprised from 5.8 to 8.5 and, from 5 to 100 mg/mL of albiglutide.

In one embodiment, compositions according to the invention comprise 100 U/mL of basal insulin which isoelectric point is comprised from 5.8 to 8.5 and, from 0.1 to 10 mg/mL of dulaglutide.

In one embodiment, compositions according to the invention comprise 40 U/mL of basal insulin which isoelectric point is comprised from 5.8 to 8.5 and, from 0.04 to 0.5 mg/mL of exenatide.

In one embodiment, compositions according to the invention comprise 40 U/mL of basal insulin which isoelectric point is comprised from 5.8 to 8.5 and, from 1 to 10 mg/mL of liraglutide.

In one embodiment, compositions according to the invention comprise 40 U/mL of basal insulin which isoelectric point is comprised from 5.8 to 8.5 and, from 0.01 to 1 mg/mL of lixisenatide.

In one embodiment, compositions according to the invention comprise 40 U/mL of basal insulin which isoelectric point is comprised from 5.8 to 8.5 and, from 5 to 100 mg/mL of albiglutide.

In one embodiment, compositions according to the invention comprise 40

U/mL of basal insulin which isoelectric point is comprised from 5.8 to 8.5 and, from 0.1 to 10 mg/mL of dulaglutide.

In one embodiment, compositions according to the invention further comprise zinc salts in a concentration comprised between 0 and 5000 μM.

In one embodiment, compositions according to the invention further comprise zinc salts in a concentration comprised between 0 and 4000 μM.

In one embodiment, compositions according to the invention further comprise zinc salts in a concentration comprised between 0 and 3000 μM.

In one embodiment, compositions according to the invention further comprise zinc salts in a concentration comprised between 0 and 2000 μM.

In one embodiment, compositions according to the invention further comprise zinc salts in a concentration comprised between 0 and 1000 μM.

In one embodiment, compositions according to the invention further comprise zinc salts in a concentration comprised between 50 and 600 μM.

In one embodiment, compositions according to the invention further comprise zinc salts in a concentration comprised between 100 and 500 μM.

In one embodiment, compositions according to the invention further comprise zinc salts in a concentration comprised between 200 and 500 μM.

In one embodiment, compositions according to the invention further comprise buffers.

In one embodiment, compositions according to the invention comprise buffers in concentrations comprised between 0 and 100 mM.

In one embodiment, compositions according to the invention comprise buffers in concentrations comprised between 15 and 50 mM.

In one embodiment, compositions according to the invention comprise a buffer chosen in the group consisting of a phosphate buffer, the Tris (trishydroxymethylaminomethane) and the sodium citrate.

In one embodiment, the buffer is the sodium phosphate.

In one embodiment, the buffer is the Tris (trishydroxymethylaminomethane).

In one embodiment, the buffer is the sodium citrate.

In one embodiment, compositions according to the invention further comprise preservatives.

In one embodiment, preservatives are chosen in the group consisting of m-cresol and phenol, alone or in blends.

In one embodiment, the concentration of preservatives is comprised between 10 and 50 mM.

In one embodiment, the concentration of preservatives is comprised between 10 and 40 mM.

In one embodiment, compositions according to the invention further comprise a surfactant.

In one embodiment, the surfactant is chosen in the group consisting of propylene glycol and polysorbate.

Compositions according to the invention may further comprise additives such as tonicity agents.

In one embodiment, tonicity agents are chosen in the group consisting of glycerine, sodium chloride, mannitol and glycine.

Compositions according to the invention may further comprise all excipients compliant with pharmacopoeias and compatible with the insulins used in standard concentrations.

The invention also relates to a pharmaceutical formulation according to the invention, characterized in that it is obtained by drying and/or lyophilization.

In the case of local and systemic releases, suitable administration routes are intravenous, subcutaneous, intradermal or intramuscular.

Transdermal, oral, nasal, vaginal, ocular, oral and pulmonary routes of administration are also considered.

The invention also relates to single-dose formulations at pH comprised from 6.0 to 8.0 comprising a basal insulin which isoelectric point is comprised from 5.8 to 8.5.

The invention also relates to single-dose formulations at pH comprised from 6.0 to 8.0 comprising a basal insulin which isoelectric point is comprised from 5.8 to 8.5 and a prandial insulin.

The invention also relates to single-dose formulations at pH comprised from 7.0 and 7.8 comprising a basal insulin which isoelectric point is comprised from 5.8 to 8.5 and a prandial insulin.

The invention also relates to single-dose formulations at pH comprised from 6.0 to 8.0 comprising a basal insulin which isoelectric point is comprised from 5.8 to 8.5 and a gastrointestinal hormone, as previously defined. The invention also relates to single-dose formulations at pH comprised from 7.0 and 7.8 comprising a basal insulin which isoelectric point is comprised from 5.8 to 8.5 and a gastrointestinal hormone, as previously defined.

The invention also relates to single-dose formulations at pH comprised from 6.0 to 8.0 comprising a basal insulin which isoelectric point is comprised from 5.8 to 8.5, a prandial insulin and a gastrointestinal hormone, as previously defined.

The invention also relates to single-dose formulations at pH comprised from 7.0 and 7.8 comprising a basal insulin which isoelectric point is comprised from 5.8 to 8.5, a prandial insulin and a gastrointestinal hormone, as previously defined.

The invention also relates to single-dose formulations at pH comprised from 6.6 and 7.8 comprising a basal insulin which isoelectric point is comprised from 5.8 to 8.5.

The invention also relates to single-dose formulations at pH comprised from 6.6 and 7.8 comprising a basal insulin which isoelectric point is comprised from 5.8 to 8.5 and a prandial insulin.

The invention also relates to single-dose formulations at pH comprised from 6.6 and 7.8 comprising a basal insulin which isoelectric point is comprised from 5.8 to 8.5 and a gastrointestinal hormone, as previously defined.

The invention also relates to single-dose formulations at pH comprised from 6.6 and 7.8 comprising a basal insulin which isoelectric point is comprised from 5.8 to 8.5, a prandial insulin and a gastrointestinal hormone, as previously defined.

The invention also relates to single-dose formulations at pH comprised from 6.6 and 7.6 comprising a basal insulin which isoelectric point is comprised from 5.8 to 8.5.

The invention also relates to single-dose formulations at pH comprised from 6.6 and 7.6 comprising a basal insulin which isoelectric point is comprised from 5.8 to 8.5 and a prandial insulin.

The invention also relates to single-dose formulations at pH comprised from 6.6 and 7.6 comprising a basal insulin which isoelectric point is comprised from 5.8 to 8.5 and a gastrointestinal hormone, as previously defined.

The invention also relates to single-dose formulations at pH comprised from 6.6 and 7.6 comprising a basal insulin which isoelectric point is comprised from 5.8 to 8.5, a prandial insulin and a gastrointestinal hormone, as previously defined.

In one embodiment, the single-dose formulations further comprise a copolyamino acid as previously defined.

In one embodiment, formulations are in the form of an injectable solution.

In one embodiment, the basal insulin which isoelectric point is comprised from 5.8 to 8.5 is insulin glargine.

In one embodiment, the prandial insulin is human insulin.

In one embodiment, the insulin is a recombinante human insulin as described in the European Pharmacopeia and the US Pharmacopeia.

In one embodiment, the prandial insulin is chosen in the group comprising insulin lispro (Humalog®), insulin glulisine (Apidra®) and insulin aspart (NovoLog®).

In one embodiment, the prandial insulin is the insulin lispro.

In one embodiment, the prandial insulin is the insulin glulisine.

In one embodiment, the prandial insulin is insulin aspart.

In one embodiment, le GLP-1 RA, analogue or derive de GLP-1 RA is chosen in the group consisting of exenatide (Byetta®), liraglutide (Victoza), lixisenatide (Lyxumia®), albiglutide (Tanzeum®), dulaglutide (Trulicity®) or one of their derivatives.

In one embodiment, the gastrointestinal hormone is exenatide.

In one embodiment, the gastrointestinal hormone is liraglutide.

In one embodiment, the gastrointestinal hormone is lixisenatide.

In one embodiment, the gastrointestinal hormone is albiglutide.

In one embodiment, the gastrointestinal hormone is dulaglutide.

The solubilization at pH from 6.0 to 8.0 of the basal insulins which isoelectric point is from 5.8 to 8.5, by the copolyamino acids bearing carboxylate charges and at least one hydrophobic radical according to the invention, may be observed and controlled simply, with the naked eye, by means of a change of appearance of the solution.

The solubilization at pH from 6.6 to 7.8 of the basal insulins which isoelectric point is from 5.8 to 8.5, by the copolyamino acids bearing carboxylate charges and at least one hydrophobic radical according to the invention, may be observed and controlled simply, with the naked eye, by means of a change of appearance of the solution.

Furthermore, and just as importantly, the applicant has been able to verify that a basal insulin whose isoelectric point is comprised from 5.8 to 8.5, solubilized at a pH from 6.0 to 8.0 in the presence of a copolyamino acid bearing carboxylate charges and at least one hydrophobic radical according to the invention, preserves its slow-acting insulin action, whether alone or in combination with a prandial insulin or a gastrointestinal hormone.

The applicant was also able to verify that a prandial insulin mixed at pH from 6.0 to 8.0 in the presence of a copolyamino acid bearing carboxylate charges and at least one hydrophobic radical according to the invention and a basal insulin whose isoelectric point is comprised from 5.8 to 8.5, preserves its rapid-release insulin action.

It is advantageously possible to prepare a composition according to the invention by simply mixing an aqueous solution of basal insulin whose isoelectric point is comprised from 5.8 to 8.5 and a copolyamino acid bearing carboxylate charges and at least one hydrophobic radical according to the invention, in aqueous solution or in lyophilized form. If necessary, the pH of the preparation is adjusted to a pH from 6.0 to 8.0.

It is advantageously possible to prepare a composition according to the invention by simply mixing an aqueous solution of basal insulin whose isoelectric point is comprised from 5.8 to 8.5 and a solution of prandial insulin, and a copolyamino acid bearing carboxylate charges and at least one hydrophobic radical according to the invention, in aqueous solution or in lyophilized form. If necessary, the pH of the preparation is adjusted to a pH from 6.0 to 8.0.

It is advantageously possible to prepare a composition according to the invention by simply mixing an aqueous solution of basal insulin whose isoelectric point is comprised from 5.8 to 8.5, and a solution of GLP-1 RA, an analog or derivative of GLP-1 RA and a co-polyamino acid bearing carboxylate charges and at least one hydrophobic radical according to the invention, in aqueous solution or in lyophilized form. If necessary, the pH of the preparation is adjusted to a pH from 6.0 to 8.0.

It is advantageously possible to prepare a composition according to the invention by simply mixing an aqueous solution of basal insulin whose isoelectric point is comprised from 5.8 to 8.5 and a solution of prandial insulin, and a solution of GLP-1 RA or an analogue or derivative of GLP-1 RA, and a co-polyamino acid bearing carboxylate charges and at least one hydrophobic radical according to the invention, in aqueous solution or in lyophilized form. If necessary, the pH of the preparation is adjusted to a pH from 6.0 to 8.0.

In one embodiment, the mixture of basal insulin and co-polyamino acid is concentrated by ultrafiltration before mixing with the prandial insulin in aqueous solution or in lyophilized form.

If necessary, the composition of the mixture is adjusted with excipients such as glycerine, m-cresol, zinc chloride and polysorbate (Tween®) by addition of concentrated solutions of these excipients to the mixture. If necessary, the pH of the preparation is adjusted to a pH from 6.0 to 8.0.

During synthesis intermediate Hy compounds and for the grafting process classical protection and deprotection methods are used:
the one or several carboxylic function(s) of Hy may be protected before grafting on PLG via an acid protecting group, this protection is achieved for example by esterification using methanol, ethanol, alcool benzylic or tert-butanol. After grafting, the functions are deprotected, that is a deprotection reaction is carried out so the carboxylic function(s) is (are) free or in the form of alkaline cation chosen in the group consisting of Na+ and K+.
the one or several amine function(s) may be protected before grafting on PLG via an amine protecting group, this protection is achieved for example by acid or basic hydrolysis under heating via the phenylmethoxycarbonyl group or the 1,1-dimethylethoxycarbonyl group. Apres the greffage, After grafting, the functions are deprotected, that is a deprotection reaction is carried out so the amine function(s) is (are) free.

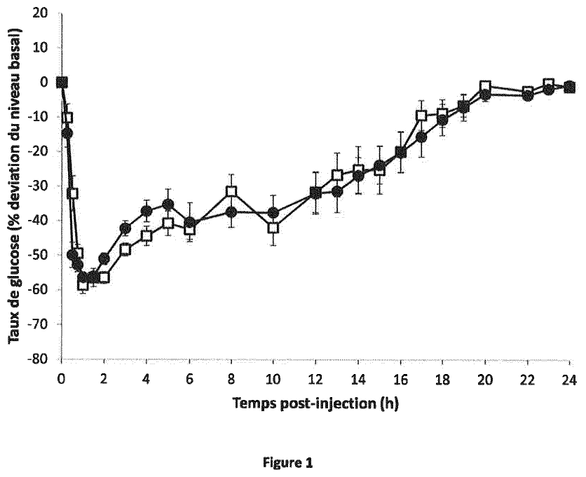

### FIG. 1

FIG. 1 depicts the glycemia average curves in percent of deviation with respect to the basal level±standard error of the average after simultaneous and separated administrations of Humalog® (100 IU/mL, 0.17 IU/kg) and Lantus® (100 IU/mL, 0.50 U/kg) (filled circles) and of the composition CB3-10 (266 U/ml, 0.67 U/kg) (empty squares); administrations have been carried out on dogs (n=10), by subcutaneous injection.

### EXAMPLES

The invention is described in more details with the following examples in a non-limited manner.

Part A—Synthesis of Intermediate Hydrophobobic Compounds Hvd that Allow Obtaining Radicals -Hy

The intermediate hydrophobobic compounds bound to spacer are represented in Table 1 by the corresponding hydrophobobic molecule before grafting on copolyamino acid.

TABLE 1
List of intermediate hydrophobic compounds bound to spacer.

| Hydrophobobic | |
|---|---|
| molecule | INTERMEDIATE HYDROPHOBIC COMPOUNDS |
| A1 | 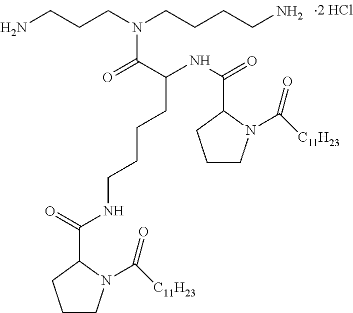 |
| A2 | 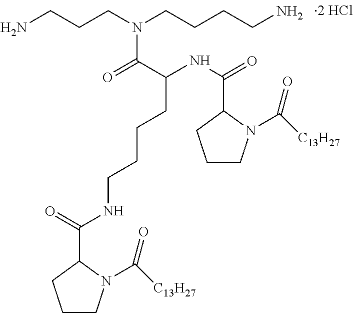 |
| A3 | 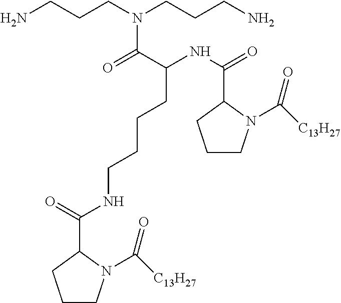 |
| A4 | 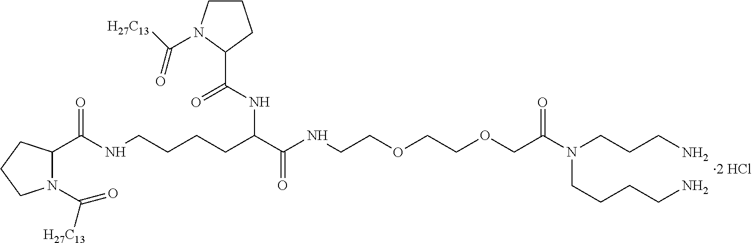 |
| A5 | 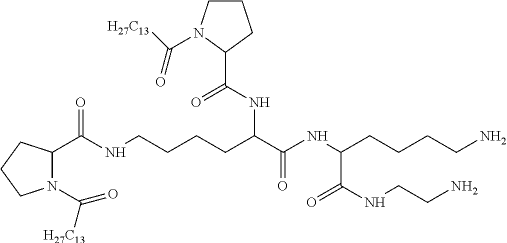 |
| A6 | 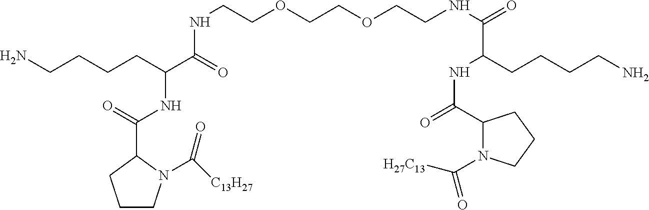 |
| A7 | 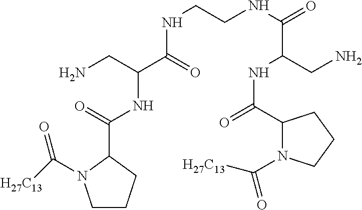 |
| A8 | 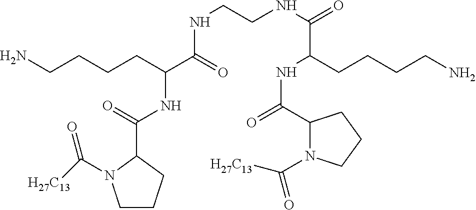 |
| A9 | 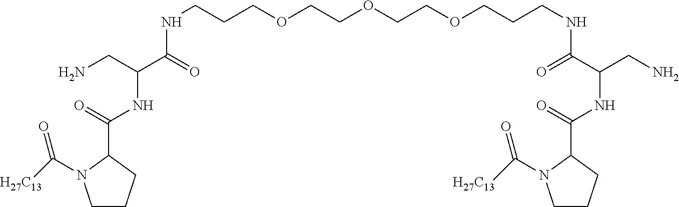 |
| A10 | 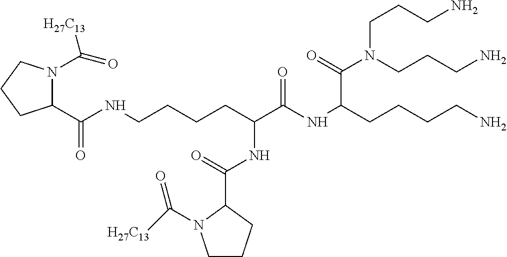 |
| A11 | 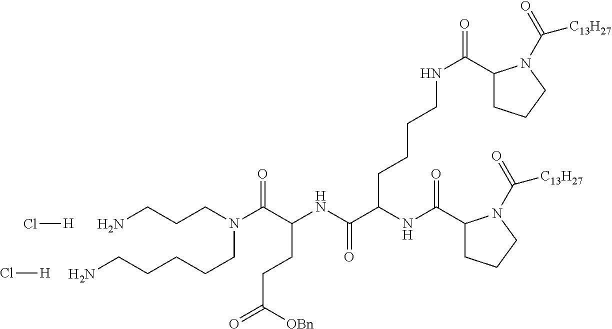 |
| A12 | 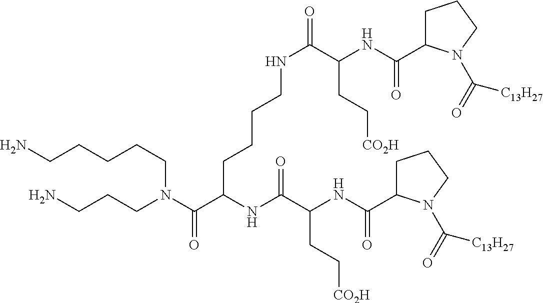 |

### Example A1—Molecule A1

### Molecule 1: Product Obtained by Coupling Between Lauric Acid and L-Proline

To a solution of lauric acid (31.63 g, 157.9 mmol) in THF (1 L) at room temperature are successively added N,N-dicyclohexylcarbodiimide (DCC) (33.24 g, 161.1 mmol) and N-hydroxysuccinimide (NHS) (18.54 g, 161.1 mmol). After stirring for 18 h at room temperature, the medium is cooled down at 0° C. over 20 min and filter on frit. L-proline (20 g, 173.72 mmol), diisopropylethylamine (DIPEA) (137.5 mL) and water (120 mL) are added to the filtrate. After stirring for 24 h at room temperature, the solvent is evapore and the residue is dissolved in water (500 mL). The aqueous phase is washed with ethyl acetate (2×500 mL), acidified until pH ˜1 with a 1 N HCl aqueous solution then extracted with dichloromethane (3×300 mL). The combined organic phases are dried over Na2SO4, filtered and concentrated under vacuum.

Yield: 34.3 g (73%)

1H NMR (CDCl₃, ppm): 0.87 (3H); 1.26 (16H); 1.70 (2H); 1.90-2.10 (3H); 2.35 (2H); 2.49 (1H); 3.48 (1H); 3.56 (1H); 4.60 (1H).

LC/MS (ESI): 298.2; (calculated ([M+H]⁺): 298.2).

### Molecule 2: Product Obtained by the Reaction Between Molecule 1 and L-Lysine

Using a similar process than the one used for preparing molecule 1 applied to molecule 1 (33.72 g, 113.36 mmol) and to L-lysine (8.70 g, 59.51 mmol), a white solid of molecule 2 is obtained.

Yield: 26.2 g (66%)

1H NMR (CDCl₃, ppm): 0.88 (6H); 1.26 (32H); 1.35-1.65 (8H); 1.85-2.35 (15H); 2.87 (1H); 3.40-3.75 (5H); 4.50-4.75 (3H); 7.87 (1H).

LC/MS (ESI): 705.6; (calculated ([M+H]⁺): 705.6).

Molecule 3: Product Obtained by the Reaction Between Spermidine and the 2-(tert-butoxycarbonyloxyimino)-2-phenylacetonitrile (Boc-ON)

To a solution of spermidine (4.07 g, 28.00 mmol) in THF (70 mL) is added triethylamine (TEA, 8.50 g, 84.01 mmol) and the reaction medium is cooled down at 0° C. A Boc-ON solution (13.66 g, 55.45 mmol) in THF (220 mL) is added dropwise over 6.5 h then the medium is stirred over 18 h at room temperature. The reaction medium is concentrated under reduced pressure, the residue is dissolved with DCM (280 mL) and the organic phase is successively washed with an 1 N aqueous soda solution (2×140 mL), water (140 mL), a saturated NaCl aqueous solution (140 mL), dried over MgSO4, filtered and concentrated under reduced pressure. A white solid of molecule 3 is obtained after solubilization of the residue in chloroform, concentration under reduced pressure and drying under vacuum.

Yield: 10.25 g (quantitative)

1H NMR (CDCl₃, ppm): 1.38-1.59 (23H); 1.61-1.70 (2H); 2.60 (2H); 2.66 (2H); 3.12 (2H); 3.20 (2H); 4.83 (1H); 5.17 (1H).

LC/MS (ESI): 346.3; (calculated ([M+H]⁺): 346.3).

### Molecule 4: Product Obtained by Coupling Between Molecule 2 and Molecule 3

To a solution of molecule 2 (5.56 g, 7.89 mmol) in chloroform (110 mL) are successively added triethylamine (1.04 g, 10.26 mmol), 1-hydroxybenzotriazole (HOBt, 1.39 g, 10.26 mmol) and N-(3-dimethylaminopropyl)-N′-ethylcarbodiimide (EDC, 1.97 g, 10.26 mmol). After 15 min, molecule 3 (3.00 g, 8.68 mmol) is added and the reaction medium is stirred over 18 h at room temperature. The organic phase is successively washed with a saturated NH4Cl aqueous solution (110 mL), a saturated NaHCO₃ aqueous solution (110 mL), a saturated NaCl aqueous solution (110 mL), dried over MgSO4, filtered and concentrated under reduced pressure. A yellowish oil of molecule 4 is obtained after purification by chromatography on silica gel (eluant: DCM, methanol).

Yield: 6.20 g (78%)

1H NMR (CD₃OD, ppm): 0.90 (6H); 1.22-2.46 (78H); 2.96-3.31 (6H); 3.31-3.76 (8H); 4.29-4.55 (2H); 4.65-4.85 (1H).

### Molecule A1

To a solution of molecule 4 (6.20 g, 6.00 mmol) in DCM (75 mL) is added a 4 N HCl in solution dioxane (15 mL) and the reaction medium is stirred over 18 h at room temperature then concentrated under reduced pressure. A white solid of molecule A1 is obtained after solubilization of the residue in DCM and concentration under reduced pressure.

Yield: 5.25 g (96%)

1H NMR (CD₃OD, ppm): 0.90 (6H); 1.21-2.46 (60H); 2.81-3.34 (6H); 3.34-3.77 (8H); 4.30-4.80 (3H).

LC/MS (ESI): 417.0; (calculated ([M+2H]²⁺): 416.9).

### Example A2—Molecule A2

### Molecule 5: Product Obtained by the Reaction Between Myristoyl Chloride and L-proline

To a solution of L-proline (300.40 g, 2.61 mol) in 2 N aqueous soda (1.63 L) at 0° C. is slowly added over 1 h myristoyl chloride (322 g, 1.30 mol) in solution in DCM (1.63 L). When completed, the reaction medium is risen up to 20° C. over 2 h then stirred for another 2 h. The mixture is cooled down at 0° C. and a 37% HCl solution (215 mL) is added over 15 min. The reaction medium is stirred for 10 min at 0° C. then over 1 h between 0° C. and 20° C. The organic phase is separated, washed with a 10% HCl solution (3×430 mL), a saturated NaCl aqueous solution (430 mL), dried over Na₂SO₄, filtered on cotton then concentrated under reduced pressure. The residue is dissolved in heptane (315 mL) then pentane (1.6 L) is added under mechanical stirring. A white solid is obtained after the filtration on frit and drying under reduced pressure.

Yield: 410.6 g (97%)

1H NMR (CDCl₃, ppm): 0.88 (3H); 1.28 (20H); 1.70 (2H); 1.90-2.10 (3H); 2.36 (2H); 2.51 (1H); 3.47 (1H); 3.56 (1H); 4.61 (1H).

LC/MS (ESI): 326.4; 651.7; (calculated ([M+H]⁺): 326.3; ([2M+H]⁺): 651.5).

### Molecule 6: Product Obtained by the Reaction Between Molecule 5 and L-Lysine

Using a similar process than the one used for preparing molecule 2 and applied to molecule 5 (20.02 g, 61.5 mmol) and to L-lysine (4.72 g, 32.29 mmol), a white solid of molecule 6 is obtained after recristallization in ethyl acetate.

Yield: 12.30 g (53%)

1H NMR (DMSO-d₆, ppm): 0.85 (6H); 1.26 (40H); 1.35-1.50 (6H); 1.50-2.10 (10H); 2.10-2.25 (4H); 3.01 (2H); 3.31-3.55 (4H); 4.10-4.40 (3H); 7.68 (0.6H); 7.97 (1H); 8.27 (0.4H); 12.50 (1H).

LC/MS (ESI): 761.8; (calculated ([M+H]⁺): 761.6).

### Molecule 7: Product Obtained by the Reaction Between Molecule 3 and Molecule 6

Using a similar process than the one used for preparing molecule 4 and applied to molecule 3 (3.00 g, 8.68 mmol) and to molecule 6 (6.00 g, 7.89 mmol), a colorless oil of molecule 7 is obtained.

Yield: 5.71 g (66%)

1H NMR (CD₃OD, ppm): 0.90 (6H); 1.23-2.48 (86H); 2.96-3.75 (14H); 4.30-4.56 (2H); 4.65-4.88 (1H).

### Molecule A2

Using a similar process than the one used for preparing molecule A1 and applied to molecule 7 (5.71 g, 5.24 mmol), a colorless oil of molecule A2 is obtained.

Yield: 5.19 g (99%)

1H NMR (CD₃OD, ppm): 0.90 (6H); 1.21-2.46 (68H); 2.81-3.32 (6H); 3.32-3.78 (8H); 4.31-4.82 (3H).

LC/MS (ESI): 445.1; (calculated ([M+2H]²⁺): 444.9).

### Example A3—Molecule A3

Molecule 8: Product Obtained by the Reaction Between Norspermidine and the tert-butylphenylcarbonate

To a solution of norspermidine (15 mL, 107.22 mmol) in DMF (70 mL) is slowly added a solution of tert-butylphenylcarbonate (49.6 mL, 268.06 mmol) in DMF (37 mL) and the mixture is stirred at room temperature over 16 h. The reaction medium is concentrated under reduced pressure, the residue is taken up in water (200 mL) then acidified until pH 1.4 with a 10% HCl aqueous solution. The aqueous phase is washed with methyl tert-butylether (MTBE, 2×500 mL), basified until pH 12 with a 10% soda aqueous solution, and the product is extracted with DCM (4×250 mL). Combined organic phases are dried over Na2SO4, filtered and concentrated under reduced pressure. A white solid of molecule 8 is obtained after cristallization in heptane.

Yield: 27.97 g (79%)

1H NMR (CDCl₃, ppm): 1.44 (18H); 1.64 (4H); 2.64 (4H); 3.20 (4H); 5.16 (2H)

LC/MS (ESI): 332.3; (calculated ([M+H]⁺): 332.3).

### Molecule 9: Product Obtained by the Reaction Between Molecule 6 and Molecule 8

Using a similar process than the one used for preparing molecule 4 and applied to molecule 6 (18.00 g, 23.65 mmol) and to molecule 8 (9.41 g, 28.38 mmol), a colorless oil of molecule 9 is obtained.

Yield: 22.83 g (90%)

1H NMR (DMSO-d₆, ppm): 0.85 (6H); 1.12-2.02 (80H); 2.02-2.30 (4H); 2.77-3.60 (14H); 4.16-4.41 (2H); 4.45-4.62 (1H); 6.59-6.94 (2H); 7.60-8.38 (2H).

LC/MS (ESI): 1075.3; (calculated ([M+H]⁺): 1074.9).

### Molecule A3

Using a similar process than the one used for preparing molecule A1 and applied to molecule 9 (22.80 g, 21.22 mmol), the obtained residue is dissolved in DCM, the organic phase is washed with a 2 N aqueous soda solution, dried over Na2SO4, filtered and concentrated under vacuum. A colorless solid of molecule A3 is obtained after solubilization of the residue in water (1.5 L) and lyophilization.

Yield: 17.76 g (96%)

1H NMR (DMSO-d6, ppm): 0.85 (6H); 1.13-2.28 (70H); 2.42-2.61 (4H); 2.83-3.61 (10H); 4.15-4.43 (2H); 4.52-4.73 (1H); 7.63-8.34 (2H).

LC/MS (ESI): 438.0, 874.9; (calculated ([M+2H]²⁺): 437.9, ([M+H]⁺): 874.7).

### Example A4: Molecule A4

Molecule 10: Product Obtained by the Reaction Between Molecule 6 and the [2-(2-aminoethoxy)ethoxy]acetic Acid

To a solution of molecule 6 (8.08 g, 10.61 mmol) in anhydrous DMF (65 mL) are added NHS (1.23 g, 10.72 mmol) then DCC (2.21 g, 10.72 mmol) and the medium is stirred at room temperature over 18 h. The mixture is filtered on frit then slowly added over a [2-(2-aminoethoxy)ethoxy]acetic acid solution (1.78 g, 10.91) in suspension in DMF (50 mL). After stirring for 24 h at room temperature, ethyl acetate (220 mL) and a 0.1 N HCl aqueous solution (100 mL) are added. The organic phase is separated, washed with a saturated NaCl aqueous solution, dried over Na2SO4, filtered and concentrated under vacuum. A colorless oil of molecule 10 is obtained after purification by flash chromatography (eluant: DCM, methanol).

Yield: 4.90 g (51%)

1H NMR (CD₃OD, ppm): 0.90 (6H); 1.22-2.49 (62H); 3.09-3.29 (2H); 3.32-3.46 (2H); 3.46-3.61 (4H); 3.61-3.72 (6H); 4.13 (2H); 4.28-4.55 (3H); 7.83-8.26 (1H).

LC/MS (ESI): 906.8; (calculated ([M+H]⁺): 906.7).

### Molecule 11: Product Obtained by the Reaction Between Molecule 3 and Molecule 10

Using a similar process than the one used for preparing molecule 4 applied to molecule 3 (2.24 g, 6.49 mmol) and to molecule 10 (4.90 g, 5.41 mmol) in solution in DMF (15 mL), a colorless oil of molecule 11 is obtained after purification by flash chromatography (eluant: DCM, methanol).

Yield: 5.30 g (79%)

1H NMR (CDCl₃, ppm): 0.88 (6H); 1.20-2.37 (86H); 2.99-3.52 (14H); 3.52-3.72 (8H); 4.14-4.26 (2H); 4.35-4.57 (3H); 4.78-5.75 (2H); 7.00-7.57 (3H).

### Molecule A4

Using a similar process than the one used for preparing molecule A1 and applied to molecule 11 (5.30 g, 4.29 mmol), a colorless oil of molecule A4 is obtained.

Yield: 4.30 g (99%)

1H NMR (CDCl₃, ppm): 0.88 (6H); 1.16-2.62 (68H); 2.95-3.93 (22H); 4.30-4.79 (5H); 7.52-8.67 (9H).

LC/MS (ESI): 517.8, 1033.8; (calculated ([M+2H]²⁺): 517.4, ([M+H]⁺): 1033.8).

### Example A5: Molecule A5

Molecule 12: Product Obtained by the Reaction Between Molecule 6 and N-epsilon-tert-butyloxycarbonyl-L-Lysine Methyl Ester (HLys(Boc)OMe)

To a solution of molecule 6 (43.00 g, 56.49 mmol) in THF (375 mL) are added at 0° C. DIPEA (8.76 g, 67.79 mmol), HOBt (865 mg, 5.65 mmol) and EDC (11.91 g, 62.14 mmol). After stirring for 15 min HLys(Boc)OMe (20.12 g, 67.79 mmol) is added and the reaction medium is stirred over 24 h at 0° C. The residue is concentrated under reduced pressure, taken up in ethyl acetate (300 mL) and the organic phase is washed with a saturated NaHCO3 aqueous solution (150 mL), a 5% HCl aqueous solution (2×150 mL) then a saturated NaCl aqueous solution (2×150 mL). After drying over Na2SO4 and filtration, the medium is concentrated under reduced pressure. A transparent solid of molecule 12 is obtained.

Yield: 55.80 g (98%)

1H NMR (CDCl₃, ppm): 0.87 (6H); 1.18-1.74 (61H); 1.74-2.07 (8H); 2.07-2.38 (8H); 2.96-3.17 (3H); 3.30-3.50 (3H); 3.54-3.67 (2H); 3.71 (3H); 4.25-4.40 (1H); 4.44-4.63 (3H); 4.74-4.98 (1H); 7.10 (1H); 7.48 (1H); 7.65 (1H).

LC/MS (ESI): 1003.8 (calculated ([M+H]⁺): 1003.8).

### Molecule 13: Product Obtained by Saponification of the Methy Ester of Molecule 12

A solution of molecule 12 (55.8 g, 55.61 mmol) in THF/methanol 1:1 (370 mL) is cooled down at 0° C., then a LiOH solution (2.0 g, 83.41 mmol) in water (185 mL) is slowly added. The reaction medium is stirred over 16 h at 0° C. then 30 min at room temperature. The residue is concentrated under reduced pressure, taken up in DCM (500 mL) and acidified with a 10% HCl aqueous solution until pH 1. DCM (500 mL) is added, the organic phase is separated and the aqueous phase is extracted with DCM (2×300 mL). Combined organic phases are washed with a saturated NaCl aqueous solution (2×300 mL), dried over Na2SO4, filtered and concentrated under reduced pressure. A white solid of molecule 13 is obtained after cristallization in acwaterne.

Yield: 46.1 g (84%)

1H NMR (Pyridine-d₅, ppm): 0.88 (6H); 1.14-2.08 (67H); 2.08-2.68 (10H); 3.14-3.97 (8H); 4.55-5.22 (4H); 7.29-7.42 (1H); 8.28-8.59 (1H); 8.91-9.39 (2H).

LC/MS (ESI): 989.8 (calculated ([M+H]⁺): 989.8).

### Molecule 14: Product Obtained by the Reaction Between Molecule 13 and the N-Boc Ethylenediamine

Using a similar process than the one used for preparing molecule 10 applied to molecule 13 (14.0 g, 14.15 mmol) in solution in DCM (94 mL) and to N-Boc ethylenediamine (2.72 g, 16.98 mmol), a white solid of molecule 14 is obtained after recristallization in acetonitrile.

Yield: 13.80 g (86%)

1H NMR (DMSO-d₆, ppm): 0.85 (6H); 1.10-2.34 (86H); 2.77-3.18 (8H); 3.27-3.68 (4H); 4.00-4.46 (4H); 6.26-6.84 (2H); 7.45-8.30 (4H).

LC/MS (ESI): 1131.8 (calculated ([M+H]⁺): 1131.9).

### Molecule A5

Using a similar process than the one used for preparing molecule A1 and applied to molecule 14 (13.80 g, 12.19 mmol), the obtained residue is dissolved in DCM, the organic phase is washed with a 2 N soda aqueous solution, dried over Na2SO4, filtered and concentrated under vacuum. A colorless solid of molecule A5 is obtained after recristallization in acetonitrile.

Yield: 9.76 g (86%)

1H NMR (DMSO-d₆, ppm): 0.85 (6H); 1.05-2.43 (72H); 2.43-2.60 (4H); 2.89-3.14 (4H); 3.28-3.64 (4H); 4.00-4.46 (4H); 7.49-8.31 (4H).

LC/MS (ESI): 466.4, 931.8 (calculated ([M+2H]²⁺): 466.4, ([M+H]⁺): 931.8).

### Example A6: Molecule A6

Molecule 15: Product Obtained by the Reaction Between Molecule 5 and the N-epsilon-tert-butyloxycarbonyl-L-Lysine (HLys(Boc)OH)

Using a similar process than the one used for preparing molecule 1 applied to molecule 5 (37.00 g, 113.67 mmol) and to the (HLys(Boc)OH) (30.80 g, 125.04 mmol), a white solid of molecule 15 is obtained after concentration of the reaction medium under reduced pressure, solubilization of the residue in water (500 mL), washing of the aqueous phase with ethyl acetate (2×250 mL) then acidification until pH 1 and filtration of the resulting precipitate.

Yield: 61.83 g (98%)

1H NMR (DMSO-d₆, ppm): 0.85 (3H); 1.13-2.05 (41H); 2.05-2.28 (2H); 2.81-2.95 (2H); 3.23-3.55 (2H); 4.07-4.15 (0.5H); 4.15-4.23 (0.5H); 4.30-4.40 (1H); 6.30-6.84 (1H); 7.99 (0.5H); 8.28 (0.5H); 12.51 (1H).

LC/MS (ESI): 554.4 (calculated ([M+H]⁺): 554.4).

Molecule 16: Product Obtained by the Reaction Between Molecule 15 and tri(ethyleneglycol)diamine

To a solution of molecule 15 (45.00 g, 81.26 mmol) in DCM (540 mL) and at 0° C. are successively added HOBt (1.24 g, 8.13 mmol), tri(ethyleneglycol)diamine (6.02 g, 40.63 mmol) then EDC (17.14 g, 89.38 mmol). The reaction mixture is stirred over 1 h at 0° C. then for 15 h at room temperature. The medium is washed with a saturated NaHCO₃ aqueous solution (2×250 mL), a 1 N HCl aqueous solution (2×250 mL), a saturated NaCl aqueous solution (250 mL), dried over Na2SO4, filtered, and concentrated under reduced pressure. A colorless solid of molecule 16 is obtained.

Yield: 47.02 g (95%)

1H NMR (DMSO-d₆, ppm): 0.85 (6H); 1.11-2.32 (86H); 2.80-2.92 (4H); 3.12-3.28 (4H); 3.28-3.59 (12H); 4.08-4.19 (1.3H); 4.19-4.34 (2H); 4.34-4.46 (0.7H); 6.32-6.81 (2H); 7.64-7.74 (1.3H); 7.74-7.83 (1.3H); 7.93-8.00 (0.7H); 8.11-8.18 (0.7H).

LC/MS (ESI): 1220.0 (calculated ([M+H]⁺): 1219.9).

### Molecule A6

Using a similar process than the one used for preparing molecule A5 and applied to molecule 16 (23.00 g, 18.86 mmol), a white solid of molecule A6 is obtained.

Yield: 16.43 g (85%)

1H NMR (DMSO-d₆, ppm): 0.85 (6H); 1.11-2.05 (68H); 2.08-2.31 (4H); 2.44-2.54 (4H); 3.10-3.58 (16H); 4.08-4.18 (1.3H); 4.22-4.32 (2H); 4.37-4.46 (0.7H); 7.66-7.87 (2.6H); 7.96-8.06 (0.7H); 8.13-8.23 (0.7H).

LC/MS (ESI): 510.5, 1020.0 (calculated ([M+2H]²⁺): 510.4, ([M+H]⁺): 1019.8).

### Example A7: Molecule A7

Molecule 17: Product Obtained by the Reaction Between Molecule 5 and N-eta-(tert-butyloxycarbonyl)-L-2.3-diaminopropionic Acid (HDap(Boc)OH)

Using a similar process than the one used for preparing molecule 1 applied to molecule 5 (100.00 g, 307.23 mmol) and to HDap(Boc)OH (65.88 g, 322.59 mmol), a white solid of molecule 17 is obtained after concentration of the organic phase under reduced pressure. The latter is directly used without any further purification.

Yield: 141.6 g (90%)

1H NMR (CDCl₃, ppm): 0.87 (3H); 1.18-1.49 (29H); 1.54-1.68 (2H); 1.82-2.41 (6H); 3.37-3.78 (4H); 4.31-4.74 (2H); 5.61 (1H); 7.63 (1H); 9.59 (1H).

LC/MS (ESI): 512.2 (calculated ([M+H]⁺): 512.4).

### Molecule 18: Product Obtained by the Reaction Between Molecule 17 and Ethylenediamine

Using a similar process than the one used for preparing molecule 16 and applied to molecule 17 (41.00 g, 80.12 mmol) and to ethylenediamine (2.41 g, 40.06 mmol) in presence of DIPEA (20.71 g, 160.25 mmol), a white solid of molecule 18 is obtained after recristallization in acetonitrile.

Yield: 40.80 g (97%)

1H NMR (DMSO-d₆, ppm): 0.85 (6H); 1.16-2.40 (74H); 2.97-3.63 (12H); 4.11-4.25 (3H); 4.25-4.41 (1H); 6.54-6.79 (2H); 7.58-8.07 (4H).

LC/MS (ESI): 1047.8 (calculated ([M+H]⁺): 1047.8).

### Molecule A7

Using a similar process than the one used for preparing molecule A5 and applied to molecule 18 (35.90 g, 34.27 mmol), a white solid of molecule A7 is obtained.

Yield: 16.43 g (85%)

1H NMR (DMSO-d₆, ppm): 0.85 (6H); 1.12-1.55 (48H); 1.67-2.31 (12H); 2.64-2.82 (4H); 2.99-3.25 (4H); 3.25-3.61 (4H); 4.02-4.12 (1.4H); 4.12-4.22 (0.6H); 4.22-4.34 (1.4H); 4.38-4.45 (0.6H); 7.70-8.20 (4H).

LC/MS (ESI): 424.4, 847.7 (calculated ([M+2H]²⁺): 424.4, ([M+H]⁺): 847.7).

### Example A8: Molecule A8

### Molecule 19: Product Obtained by the Reaction Between Molecule 15 and the Ethylenediamine

Using a similar process than the one used for preparing molecule 18 applied to molecule 15 (16.05 g, 29.0 mmol) and to ethylenediamine (0.96 g, 16.0 mmol), a white solid of molecule 19 is obtained after recristallization in acetonitrile.

Yield: 19.78 g (60%)

1H NMR (DMSO-d₆, ppm): 0.85 (6H); 1.09-2.38 (86H); 2.80-2.91 (4H); 3.00-3.60 (8H); 4.04-4.15 (1.3H); 4.15-4.23 (0.7H); 4.23-4.31 (1.3H); 4.36-4.45 (0.7H); 6.27-6.82 (2H); 7.60-8.21 (4H).

LC/MS (ESI): 1131.8, 1153.8 (calculated ([M+H]⁺): 1131.9, ([M+Na]⁺): 1153.9).

### Molecule A8

Using a similar process than the one used for preparing molecule A1 and applied to molecule 19 (19.78 g, 17.48 mmol), a white solid of molecule A8 is obtained after solubilization of the residue in water, addition of a 2 N aqueous soda solution until formation of a precipitate, filtration and drying under reduced pressure.

Yield: 9.93 g (61%)

1H NMR (CDCl₃, ppm): 0.88 (6H); 1.16-2.25 (68H); 2.25-2.39 (4H); 2.65-2.83 (4H); 3.22-3.54 (6H); 3.54-3.74 (2H); 4.23-4.37 (2H); 4.43-4.58 (2H); 7.40 (2H); 7.63 (2H).

LC/MS (ESI): 466.7, 931.8 (calculated ([M+2H]²⁺): 466.4, ([M+H]⁺): 931.8).

### Example A9: Molecule A9

Molecule 20: Product Obtained by the Reaction Between Molecule 17 and the 4,7,10-trioxa-1,13-tridecanediamine

Using a similar process than the one used for preparing molecule 18 applied to molecule 17 (32.00 g, 62.54 mmol) and to the 4,7,10-trioxa-1,13-tridecanediamine (6.89 g, 31.27 mmol), a white solid of molecule 20 is obtained.

Yield: 29.23 g (77%)

1H NMR (DMSO-d₆, ppm): 0.85 (6H); 1.17-2.38 (78H); 3.01-3.15 (4H); 3.15-3.62 (20H); 4.09-4.24 (3H); 4.28-4.40 (1H); 6.58-6.87 (2H); 7.41-8.03 (4H).

LC/MS (ESI): 1207.9 (calculated ([M+H]⁺): 1207.9).

### Molecule A9

Using a similar process than the one used for preparing molecule A5 and applied to molecule 20 (28.30 g, 23.43 mmol), a white solid of molecule A9 is obtained.

Yield: 15.76 g (67%)

1H NMR (DMSO-d₆, ppm): 0.85 (6H); 1.10-2.33 (64H); 2.60-2.86 (4H); 3.01-3.19 (4H); 3.27-3.62 (16H); 4.01-4.13 (1.4H); 4.13-4.20 (0.6H); 4.20-4.30 (1.4H); 4.36-4.46 (0.6H); 7.57-7.70 (1.4H); 7.70-7.85 (1.4H); 7.91-8.03 (0.6H); 8.03-8.19 (0.6H).

LC/MS (ESI): 504.4, 1007.9 (calculated ([M+2H]²⁺): 504.4, ([M+H]⁺): 1007.8).

### Example A10: Molecule A10

### Molecule 21: Product Obtained by the Reaction Between Molecule 8 and Molecule 13

Using a similar process than the one used for preparing molecule 4 applied to molecule 8 (7.12 g, 21.47 mmol) and to molecule 13 (17.70 g, 17.89 mmol) stirring between 0° C. and room temperature over 16 h, a white foam of molecule A10 is obtained after washing of the reaction medium in dichloromethane with a saturated NaHCO₃ aqueous solution (2×100 mL), a 10% HCl aqueous solution (2×100 mL), a saturated NaCl aqueous solution (50 mL), drying over Na2SO4, filtration and concentration under vacuum.

Yield: 20.10 g (86%)

1H NMR (DMSO-d₆, ppm): 0.85 (6H); 1.11-2.04 (95H); 2.04-2.32 (4H); 2.76-3.61 (16H); 4.09-4.62 (4H); 6.26-7.01 (3H); 7.57-8.43 (3H).

LC/MS (ESI): 1303.1, 1325.1 (calculated ([M+H]⁺): 1303.0, ([M+Na]⁺): 1325.0).

### Molecule A10

Using a similar process than the one used for preparing molecule A5 and applied to molecule 21 (20.10 g, 15.43 mmol), a pale yellow solid of molecule A10 is obtained.

Yield: 10.70 g (69%)

1H NMR (DMSO-d₆, ppm): 0.85 (6H); 1.11-2.31 (78H); 2.41-2.70 (6H); 2.87-3.68 (10H); 4.06-4.50 (3H); 4.56-4.77 (1H); 7.59-8.35 (3H).

LC/MS (ESI): 501.9, 1002.7 (calculated ([M+2H]²⁺): 501.9, ([M+H]⁺): 1002.8).

### Example A11: Molecule A11

Molecule 22: Product Obtained by the Reaction Between Molecule 6 and α-tert-butyl-γ-benzyl L-glutamate

Using a similar process than the one used for preparing molecule 12 applied to molecule 6 (5.0 g, 6.57 mmol) in solution in chloroform (34 mL) and to α-tert-butyl-γ-benzyl L-glutamate (2.167 g, 6.57 mmol), a white solid of molecule 22 is obtained after purification by flash chromatography (eluant: DCM, MeOH).

Yield: 6.16 g (90%)

1H NMR (CDCl₃, ppm): 0.88 (6H); 1.18-2.51 (75H); 2.89-3.68 (6H); 4.18-4.66 (4H); 5.11 (2H); 7.11 (1H); 7.28-7.41 (5H); 7.55 (1H); 7.70 (1H).

LC/MS (ESI): 1037.0 (calculated ([M+H]⁺): 1036.8).

### Molecule 23: Product Obtained by the Reaction Between Molecule 22 and HCl

To a solution of molecule 22 (6.16 g, 5.94 mmol) in DCM (60 mL) is added a 4 N HCl in solution dioxane (15 mL). After 48 h at room temperature, the reaction medium is concentrated under reduced pressure and the residue is purified by flash chromatography (eluant: DCM, MeOH).

Yield: 3.05 g (51%)

1H NMR (CDCl₃, ppm): 0.88 (6H); 1.14-2.58 (66H); 2.98-3.69 (6H); 4.24-4.61 (4H); 5.10 (2H); 7.27-7.40 (5H); 6.75-7.60 (3H).

LC/MS (ESI): 980.8 (calculated ([M+H]⁺): 980.7).

### Molecule 24: Product Obtained by the Reaction Between Molecule 23 and Molecule 3

Using a similar process than the one used for preparing molecule 4 applied to molecule 23 (1.5 g, 1.53 mmol) and to molecule 3 (0.582 g, 1.683 mmol) in solution in chloroform (23 mL), a colorless oil of molecule 24 is obtained after purification by flash chromatography (eluant: DCM, methanol).

Yield: 1.54 g (77%)

1H NMR (CDCl₃, ppm): 0.88 (6H); 1.18-2.53 (90H); 2.85-3.68 (14H); 4.24-4.63 (3H); 4.80-4.98 (1H); 5.06-5.21 (2H); 7.28-7.39 (5H).

### Molecule A11

Using a similar process than the one used for preparing molecule A1 and applied to molecule 24 (1.54 g, 1.18 mmol), a white solid of molecule A11 as a chlorhydrate salt is obtained.

Yield: 1.32 g (95%)

1H NMR (CD₃OD, ppm): 0.90 (6H); 1.17-2.28 (66H); 2.34-3.60 (6H); 2.84-3.07 (4H); 3.07-3.29 (2H); 3.37-3.80 (8H); 4.16-4.46 (3H); 4.84-4.98 (1H); 5.14 (2H); 7.30-7.40 (5H).

LC/MS (ESI): 554.7; 1108.0; (calculated ([M+2H]²⁺): 554.4; ([M+H]⁺): 1107.9).

### Example A12: Molecule A12

### Molecule 25: Product Obtained by the Reaction Between Fmoc-Lys(Fmoc)-OH and 2-Cl-trityl Chloride Resin

To Fmoc-Lys(Fmoc)-OH (7.32 g, 12.40 mmol) in suspension in dichloromethane (60 mL) is added DIPEA (4.32 mL, 24.80 mmol) at room temperature. After complete solubilization (10 min), the obtained solution is poured over 2-Cl-trityl chloride resin beforehand washed with dichloromethane (100-200 mesh, 1% DVB, 1.24 mmol/g) (4.00 g, 4.96 mmol), in an appropriate reactor for peptide synthesis on solid support. After stirring for 2 h at room temperature, grade HPLC methanol (0.8 mL/g resin, 3.2 mL) is added and the medium is stirred at room temperature over 15 min. The resin is filtered, successively washed with dichloromethane (3×60 mL), DMF (2×60 mL), dichloromethane (2×60 mL), isopropanol (1×60 mL) and dichloromethane (3×60 mL).

### Molecule 26: Product Obtained by Reaction Between Molecule 25 and an 80:20 DMF/Piperidine Mixture

Molecule 25, beforehand washed with DMF, is treated with an 80:20 DMF/piperidine mixture (60 mL). After stirring for 30 min at room temperature, the resin is filtered, washed successively with DMF (3×60 mL), isopropanol (1×60 mL) and dichloromethane (3×60 mL).

### Molecule 27: Product Obtained by the Reaction Between Molecule 26 and Fmoc-Glu(OtBu)-OH

To a suspension of Fmoc-Glu(OtBu)-OH (10.55 g, 24.80 mmol) and 1-[bis(dimethylamino)methylene]-1H-1,2,3-triazolo[4,5-b]pyridinium 3-oxide hexafluorophosphate (HATU, 9.43 g, 24.80 mmol) in a 1:1 DMF/dichloromethane mixture (60 mL) is added DIPEA (8.64 mL, 49.60 mmol). After complete solubilization, the obtained solution is poured over molecule 26. After stirring for 2 h at room temperature, the resin is filtered, washed successively with DMF (3×60 mL), isopropanol (1×60 mL) and dichloromethane (3×60 mL).

### Molecule 28: Product Obtained by the Reaction Between Molecule 27 and a 50:50 DMF/Morpholine Mixture

The molecule 27, beforehand washed with DMF, is treated with a 50:50 DMF/morpholine mixture (60 mL). After stirring for 1 h 15 at room temperature, the resin is filtered, washed successively with DMF (3×60 mL), isopropanol (1×60 mL) and dichloromethane (3×60 mL).

### Molecule 29: Product Obtained by the Reaction Between Molecule 28 and Molecule 5

Using a similar process than the one used for molecule 27 applied to molecule 28 and to molecule 5 (8.07 g, 24.80 mmol) in DMF (60 mL), molecule 29 is obtained.

Molecule 30: Product Obtained by the Reaction Between Molecule 29 and a 80:20 Dichloromethane/1.1.1.3.3.3-hexafluoro-2-propanol (HFIP) Mixture

Molecule 29 is treated with a 80:20 dichloromethane/1.1.1.3.3.3-hexafluoro-2-propanol (HFIP) mixture (60 mL). After stirring for 20 min at room temperature, the resin is filtered and washed with dichloromethane (2×60 mL). Solvents are evaporated under reduced pressure. Two co-evaporations are then carried out on the residue using dichloromethane (60 mL) then diisopropylether (60 mL). The product is purified by chromatography on silica gel (dichloromethane, methanol). A white solid of molecule 30 is obtained.

Yield: 2.92 g (52% over 6 steps)

1H NMR (CD₃OD, ppm): 0.90 (6H); 1.22-2.47 (88H); 3.13-3.25 (2H); 3.45-3.76 (4H); 4.24-4.55 (5H).

LC/MS (ESI+): 1131.9 (calculated ([M+H]⁺): 1131.8).

### Molecule 31: Product Obtained by the Reaction Between Molecule 30 and Molecule 3

Using a similar process than the one used for preparing molecule 4 applied to molecule 30 (3.12 g, 2.76 mmol) and to molecule 3 (1.048 g, 3.03 mmol) in solution in chloroform (40 mL), a colorless oil of molecule 31 is obtained after purification by flash chromatography (eluant: DCM, methanol).

Yield: 2.57 g (64%)

1H NMR (CD₃OD, ppm): 0.90 (6H); 1.21-2.47 (112H); 2.82-3.74 (14H); 4.16-4.53 (4H); 4.53-4.78 (1H).

### Molecule A12

Using a similar process than the one used for preparing molecule A1 and applied to molecule 31 (2.56 g, 1.75 mmol), a white solid of molecule A12 as a chlorhydrate salt is obtained. The latter is dissolved in water (40 mL) adding a 1 N NaOH solution until reaching a pH 7 then the solution is lyophilized to give a white solid of molecule A12.

Yield: 2.1 g (95%)

1H NMR (CD₃OD, ppm): 0.90 (6H); 1.19-2.32 (68H); 2.32-2.54 (8H); 2.82-3.15 (4H); 3.15-3.79 (10H); 4.23-4.76 (5H).

LC/MS (ESI): 1146.9; (calculated ([M+H]⁺): 1146.8).

### Part B—Synthesis of Hydrophobic Copolyamino Acids

The hydrophobic copolyamino acids are represented in Table 2.

TABLE 2
List of hydrophobic copolyamino acids.

| Copolyamino | |
|---|---|
| acid | Structure |
| B1 | 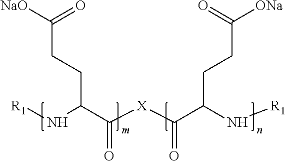 |
| | i′ = 0.022, DP (m + n) = 45 |
| | 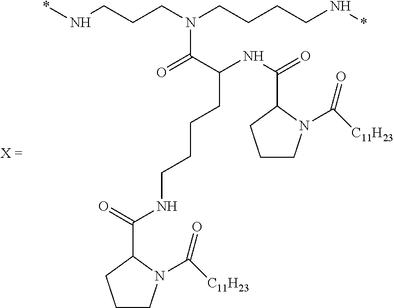 |
| | R₁ = H or pyroglutamate |
| B2 | 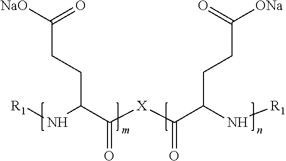 |
| | i′ = 0.045, DP (m + n) = 22 |
| | 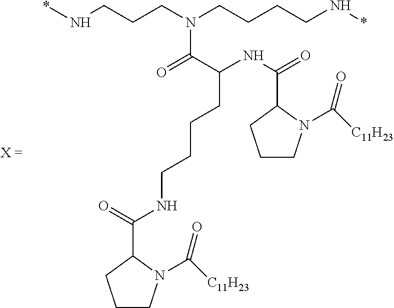 |
| | R₁ = H or pyroglutamate |
| B3 | 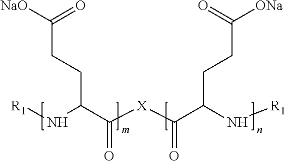 |
| | i′ = 0.05, DP (m + n) = 20 |
| | 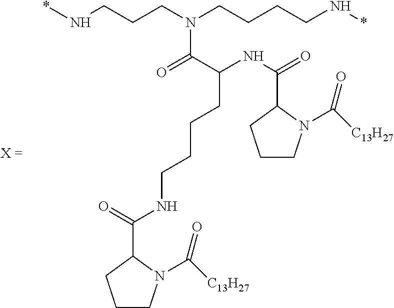 |
| | R₁ = H or pyroglutamate |
| B4 | 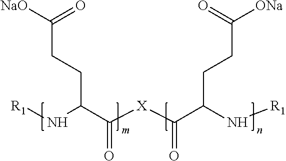 |
| | i′ = 0.022, DP (m + n) = 46 |
| | 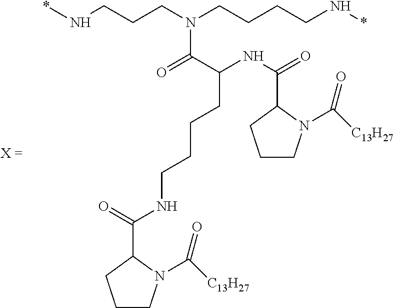 |
| | R₁ = H or pyroglutamate |
| B5 | 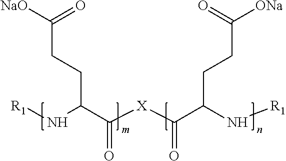 |
| | i′ = 0.015, DP (m + n) = 66 |
| | 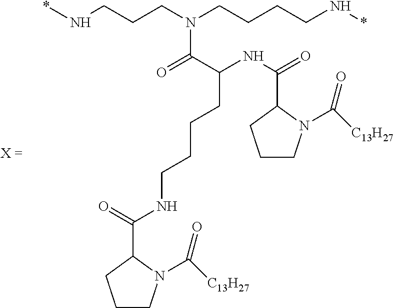 |
| | R₁ = H or pyroglutamate |
| B6 | 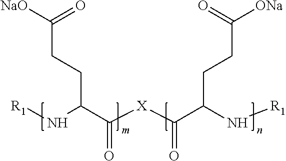 |
| | i′ = 0.056, DP (m + n) = 18 |
| | 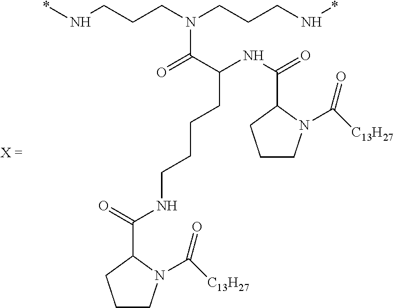 |
| | R₁ = H or pyroglutamate |
| B7 | 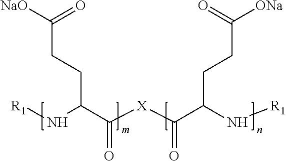 |
| | i′ = 0.043, DP (m + n) = 23 |
| | 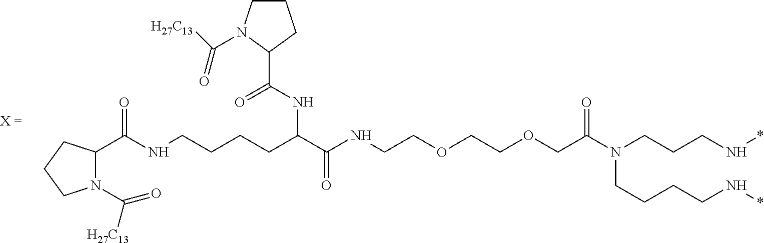 |
| | R₁ = H or pyroglutamate |
| B8 | 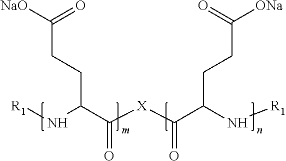 |
| | i′ = 0.05, DP (m + n) = 20 |
| | 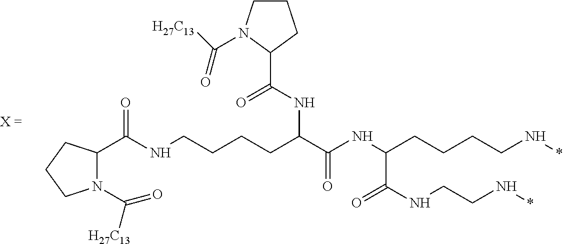 |
| | R₁ = H or pyroglutamate |
| B9 | 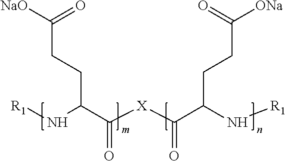 |
| | i′ = 0.05, DP (m + n) = 20 |
| | 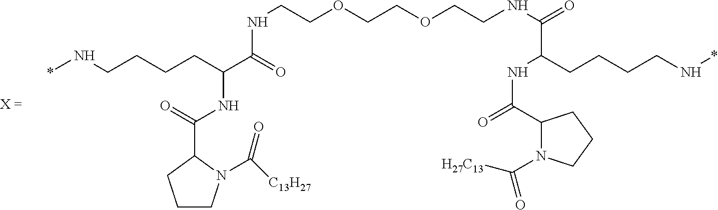 |
| | R₁ = H or pyroglutamate |
| B10 | 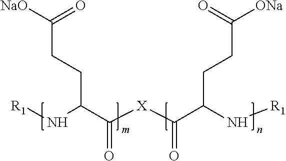 |
| | i′ = 0.05, DP (m + n) = 20 |
| | 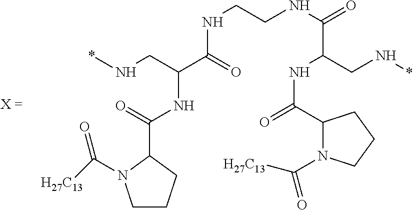 |
| | R₁ = H or pyroglutamate |
| B11 | 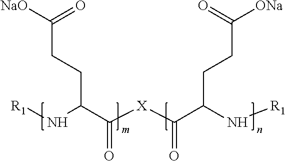 |
| | i′ = 0.042, DP (m + n) = 24 |
| | 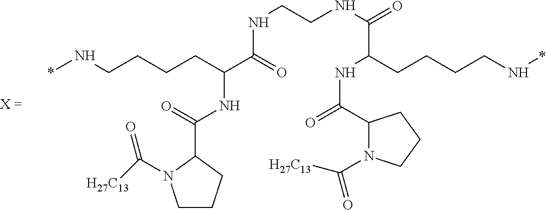 |
| | R₁ = H or pyroglutamate |
| B12 | 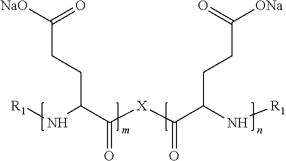 |
| | i′ = 0.05, DP (m + n) = 20 |
| | 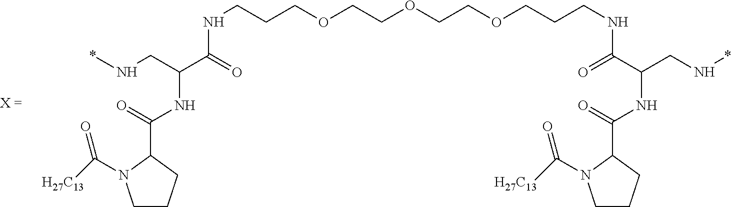 |
| | R₁ = H or pyroglutamate |
| B13 | 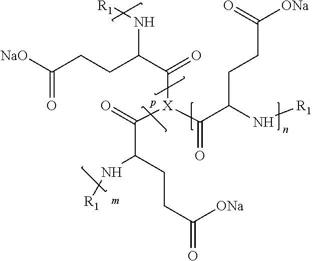 |
| | i′ = 0.037, DP (m + n + p) = 27 |
| | 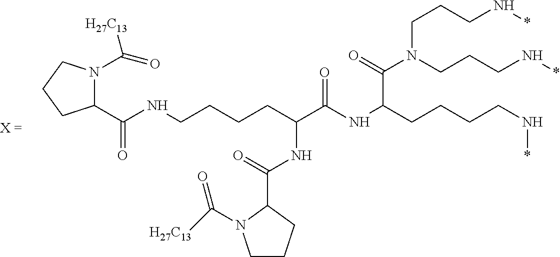 |
| | R₁ = H or pyroglutamate |
| B14 | 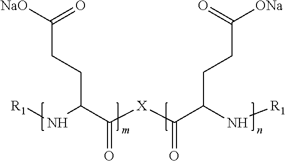 |
| | R₁ = H or pyroglutamate |
| | i′ = 0.026, DP (m + n) = 38 |
| | 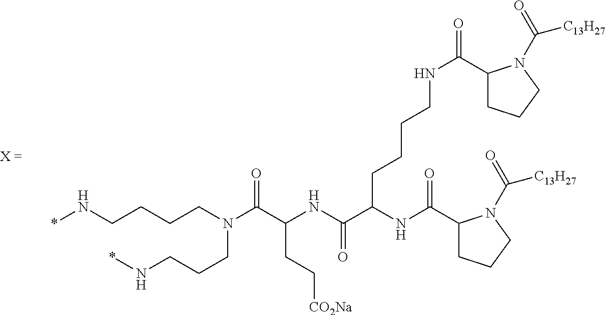 |
| B15 | 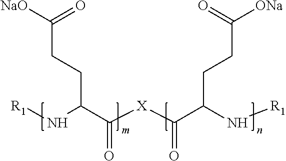 |
| | R₁ = H or pyroglutamate |
| | i′ = 0.042, DP (m + n) = 24 |
| | 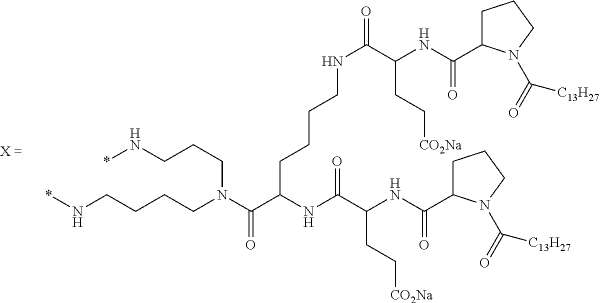 |

### Example B1

Copolyamino Acid B1—sodium poly-L-glutamate Modified with Molecule A1 and Having a Number-Average Molecular Weight (Mn) of 6850 g/mol

In an appropriate container are successively introduce the chlorhydrate salt of molecule A1 (3.70 g, 4.09 mmol), chloroform (40 mL), 4 Å molecular sieve (1.5 g), and Amberlite IRN-150 ion exchange resin (1.5 g). After stirring for 2.5 h on rolls, the medium is filtered and the resin is rinsed with chloroform. The mixture is evaporated then co-evaporated with toluene. The residue is dissolved in anhydrous DMF (5 mL) to be directly used in the next reaction.

In an oven-dried round-bottom flask, γ-benzyl-L-glutamate N-carboxyanhydride (43.04 g, 163.50 mmol) is placed under vacuum over 2 h then anhydrous DMF (80 mL) is introduced. The mixture is stirred under argon until complete solubilization, cooled down to 4° C., then the solution of molecule A1, prepared as previously described, is introduced rapidly. The mixture is stirred between 4° C. and room temperature over 2 days, then heated at 65° C. over 2 h. The reaction mixture is then cooled down at room temperature then poured dropwise in diisopropylether (3.2 L) under stirring. The white precipitate is collected by filtration, washed twice with diisopropylether (150 mL), then dried under vacuum at 30° C. to obtain a white solid. The solid is dissolved in trifluoroacetic acid (152 mL), and A 33% HBr solution in acetic acid (106 mL) is then added dropwise and at 0° C. The solution is stirred over 2 h at room temperature then poured dropwise over a 1:1 (v/v) diisopropylether/water mixture under stirring (1.8 L). After stirring for 2 h, the heterogenous mixture is left overnight. The white precipitate is collected by the filtration, washed successively with a 1:1 (v/v) diisopropylether/water mixture (150 mL), then with water (150 mL). The obtained solid is dissolved in water (750 mL), the pH is adjusted to 7.5 by addition of a 1 N aqueous soda solution, then the polymer theoritical concentration is adjusted to 20 g/L by addition of water. The mixture is filtered on 0.45 μm frit, then is purified by ultrafiltration against a 0.9% NaCl solution, then water until the conductimetry of the permeate is inferior to 50 μS/cm. The copolyamino acid solution is then concentrated to about theorical 30 g/L and the pH is adjusted to 7.0. The aqueous solution is filtered on 0.2 μm frit and kept at 4° C.

Dry extract: 25.8 mg/g
DP (estimated by 1H NMR)=45 that is a ratio j/(m+n)=0.022, said ratio j/(m+n) being called i′.
The calculated number-average molecular weight of copolyamino acid B1 is 7552 g/mol.
Aqueous HPLC-SEC (calibrant PEG): Mn=6850 g/mol.

### Example B2

Copolyamino Acid B2—Sodium poly-L-glutamate Modified with Molecule A1 and Having a Number-Average Molecular Weight (Mn) of 3330 g/mol

Using a similar process than the one used for preparing copolyamino acid B1 applied to the chlorhydrate salt of molecule A1 (4.76 g, 5.26 mmol) and to γ-benzyl-L-glutamate N-carboxyanhydride (27.68 g, 105.1 mmol), a sodium poly-L-glutamate modified with molecule A1 is obtained.

Dry extract: 25.6 mg/g
DP (estimated by 1H NMR)=22 and i′=0.045
The calculated number-average molecular weight of copolyamino acid B2 is 4076 g/mol.
Aqueous HPLC-SEC (calibrant PEG): Mn=3330 g/mol.

### Example B3

Copolyamino Acid B3—Sodium poly-L-glutamate Modified with Molecule A2 and Having a Number-Average Molecular Weight (Mn) of 3360 g/Mol

Using a similar process than the one used for preparing copolyamino acid B1 applied to the chlorhydrate salt of molecule A2 (4.90 g, 5.10 mmol) and to γ-benzyl-L-glutamate N-carboxyanhydride (26.83 g, 101.9 mmol), a sodium co-poly-L-glutamate modified with molecule A2 is obtained.

Dry extract: 29.7 mg/g
DP (estimated by 1H NMR)=20 and i′=0.05
The calculated number-average molecular weight of copolyamino acid B3 is 3830 g/mol.
Aqueous HPLC-SEC (calibrant PEG): Mn=3360 g/mol.

### Example B4

Copolyamino Acid B4—Sodium poly-L-glutamate Modified with Molecule A2 and Having a Number-Average Molecular Weight (Mn) of 7050 g/Mol

Using a similar process than the one used for preparing copolyamino acid B1 applied to the chlorhydrate salt of molecule A2 (5.90 g, 6.14 mmol) and to γ-benzyl-L-glutamate N-carboxyanhydride (64.63 g, 245.50 mmol), a sodium co-poly-L-glutamate modified with molecule A2 is obtained.

Dry extract: 24.1 mg/g
DP (estimated by 1H NMR)=46 and i′=0.022
The calculated number-average molecular weight of copolyamino acid B4 is 7759 g/mol.
Aqueous HPLC-SEC (calibrant PEG): Mn=7050 g/mol.

### Example B5

Copolyamino Acid B5—Sodium poly-L-glutamate Modified with Molecule A2 and Having a Number-Average Molecular Weight (Mn) of 10440 g/Mol

Using a similar process than the one used for preparing copolyamino acid B1 applied to the chlorhydrate salt of molecule A2 (3.09 g, 3.21 mmol) and to γ-benzyl-L-glutamate N-carboxyanhydride (52.09 g, 197.9 mmol), a sodium co-poly-L-glutamate modified with molecule A2 is obtained.

Dry extract: 27.2 mg/g
DP (estimated by 1H NMR)=66 and i′=0.015
The calculated number-average molecular weight of copolyamino acid B5 is from 10781 g/mol.
Aqueous HPLC-SEC (calibrant PEG): Mn=10440 g/mol.

### Example B6

Copolyamino Acid B6—Sodium poly-L-glutamate Modified with Molecule A3 and Having a Number-Average Molecular Weight (Mn) of 3020 g/Mol

Using a similar process than the one used for preparing copolyamino acid B1 applied to molecule A3 as a free amine (4.15 g, 4.75 mmol) and to γ-benzyl-L-glutamate N-carboxyanhydride (25.0 g, 95.0 mmol), a sodium co-poly-L-glutamate modified with molecule A3 is obtained.

Dry extract: 20.4 mg/g
DP (estimated by 1H NMR)=18 and i′=0.056
The calculated number-average molecular weight of copolyamino acid B6 is 3514 g/mol.
Aqueous HPLC-SEC (calibrant PEG): Mn=3020 g/mol.

### Example B7

Copolyamino Acid B7—Sodium poly-L-glutamate Modified with Molecule A4 and Having a Number-Average Molecular Weight (Mn) of 3360 g/Mol

Using a similar process than the one used for preparing copolyamino acid B1 applied to molecule A4 as a free amine (2.06 g, 1.99 mmol) and to γ-benzyl-L-glutamate N-carboxyanhydride (10.5 g, 39.9 mmol), a sodium co-poly-L-glutamate modified with molecule A4 is obtained.

Dry extract: 17.6 mg/g
DP (estimated by 1H NMR)=23 and i′=0.043
The calculated number-average molecular weight of copolyamino acid B7 is 4429 g/mol.
Aqueous HPLC-SEC (calibrant PEG): Mn=3360 g/mol.

### Example B8

Copolyamino Acid B8—Sodium poly-L-glutamate Modified with Molecule A5 and Having a Number-Average Molecular Weight (Mn) of 3360 g/Mol

Using a similar process than the one used for preparing copolyamino acid B1 applied to molecule A5 as a free amine (3.89 g, 4.18 mmol) and to γ-benzyl-L-glutamate N-carboxyanhydride (22.00 g, 84.00 mmol), a sodium co-poly-L-glutamate modified with molecule A5 is obtained.

Dry extract: 17.9 mg/g
DP (estimated by 1H NMR)=20 and i′=0.050
The calculated number-average molecular weight of copolyamino acid B8 is 3873 g/mol.
Aqueous HPLC-SEC (calibrant PEG): Mn=3360 g/mol.

### Example B9

Copolyamino Acid B9—Sodium poly-L-glutamate Modified with Molecule A6 and Having a Number-Average Molecular Weight (Mn) of 3340 g/Mol

Using a similar process than the one used for preparing copolyamino acid B1 applied to molecule A6 as a free amine (5.81 g, 5.70 mmol) and to γ-benzyl-L-glutamate N-carboxyanhydride (30.00 g, 114 mmol), a sodium co-poly-L-glutamate modified with molecule A6 is obtained.

Dry extract: 16.4 mg/g
DP (estimated by 1H NMR)=20 donc i=0.05
The calculated number-average molecular weight of copolyamino acid B9 is 3961 g/mol.
Aqueous HPLC-SEC (calibrant PEG): Mn=3340 g/mol.

### Example B10

Copolyamino Acid B10—Sodium poly-L-glutamate Modified with Molecule A7 and Having a Number-Average Molecular Weight (Mn) of 2920 g/Mol

Using a similar process than the one used for preparing copolyamino acid B1 applied to molecule A7 as a free amine (3.54 g, 4.18 mmol) and to γ-benzyl-L-glutamate N-carboxyanhydride (22.00 g, 84.00 mmol), a sodium co-poly-L-glutamate modified with molecule A7 is obtained.

Dry extract: 18.4 mg/g
DP (estimated by 1H NMR)=20 and i′=0.05
The calculated number-average molecular weight of copolyamino acid B10 is 3789 g/mol.
Aqueous HPLC-SEC (calibrant PEG): Mn=2920 g/mol.

### Example B11

Copolyamino Acid B11—Sodium poly-L-glutamate Modified with Molecule A8 and Having a Number-Average Molecular Weight (Mn) of 3750 g/Mol

Using a similar process than the one used for preparing copolyamino acid B1 applied to molecule A8 as a free amine (2.51 g, 2.69 mmol) and to γ-benzyl-L-glutamate N-carboxyanhydride (14.19 g, 53.90 mmol), a sodium co-poly-L-glutamate modified with molecule A8 is obtained.

Dry extract: 20.9 mg/g
DP (estimated by 1H NMR)=24 and i′=0.042
The calculated number-average molecular weight of copolyamino acid B11 is 4478 g/mol.
Aqueous HPLC-SEC (calibrant PEG): Mn=3750 g/mol.

### Example B12

Copolyamino Acid B12—Sodium poly-L-glutamate Modified with Molecule A9 and Having a Number-Average Molecular Weight (Mn) of 3660 g/Mol

Using a similar process than the one used for preparing copolyamino acid B1 applied to molecule A9 as a free amine (4.21 g, 4.18 mmol) and to γ-benzyl-L-glutamate N-carboxyanhydride (22.00 g, 84.00 mmol), a sodium co-poly-L-glutamate modified with molecule A9 is obtained.

Dry extract: 18.6 mg/g
DP (estimated by 1H NMR)=20 and i′=0.050
The calculated number-average molecular weight of copolyamino acid B12 is 3949 g/mol.
Aqueous HPLC-SEC (calibrant PEG): Mn=3660 g/mol.

### Example B13

Copolyamino Acid B13—Sodium poly-L-glutamate Modified with Molecule A10 and Having a Number-Average Molecular Weight (Mn) of 4170 g/Mol

Using a similar process than the one used for preparing copolyamino acid B1 applied to molecule A10 as a free amine (3.81 g, 3.80 mmol) and to γ-benzyl-L-glutamate N-carboxyanhydride (30.00 g, 114.00 mmol), a sodium co-poly-L-glutamate modified with molecule A10 is obtained.

Dry extract: 22.3 mg/g
DP (estimated by 1H NMR)=27 and i′=0.037
The calculated number-average molecular weight of copolyamino acid B13 is 4962 g/mol.
Aqueous HPLC-SEC (calibrant PEG): Mn=4170 g/mol.

### Example B14

Copolyamino Acid B14—Sodium poly-L-glutamate Modified with Molecule all and Having a Number-Average Molecular Weight (Mn) of 6500 g/Mol

Using a similar process than the one used for preparing copolyamino acid B1 applied to chlorhydrate molecule A11 (1.21 g, 1.09 mmol) and to γ-benzyl-L-glutamate N-carboxyanhydride (10.8 g, 41.0 mmol), a sodium co-poly-L-glutamate modified with molecule A11 is obtained.

Dry extract: 22.2 mg/g
DP (estimated by 1H NMR)=38 and i′=0.026
The calculated number-average molecular weight of copolyamino acid B14 is 6701 g/mol.
Aqueous HPLC-SEC (calibrant PEG): Mn=6500 g/mol.

### Example B15

Copolyamino Acid B15—Sodium poly-L-glutamate Modified with Molecule A12 and Having a Number-Average Molecular Weight (Mn) of 4300 g/Mol

Using a similar process than the one used for preparing copolyamino acid B1 applied to molecule A12 as a free amine (2.00 g, 1.58 mmol) and to γ-benzyl-L-glutamate N-carboxyanhydride (8.34 g, 31.7 mmol), a sodium co-poly-L-glutamate modified with molecule A12 is obtained.

Dry extract: 15.1 mg/g
DP (estimated by 1H NMR)=24 and i′=0.042
The calculated number-average molecular weight of copolyamino acid B15 is 4737 g/mol.
Aqueous HPLC-SEC (calibrant PEG): Mn=4300 g/mol.

### Part C—Commercial Compositions

### Example C1: Fast-Acting Insulin Analog Solution (Humalog®) at 100 U/mL

This solution is a commercial solution of insulin lispro, marketed by ELI

LILLY under the name Humalog®. This product is a fast-acting insulin analog. The excipients in Humalog® are meta-cresol (3.15 mg/mL), glycerol (16 mg/mL), disodium phosphate (1.88 mg/mL), zinc oxide (to have 0.0197 mg of zinc ion/mL), sodium hydroxide and hydrochloric acid to adjust the pH (pH 7-7.8) and water.

### Example C2: Solution of Rapid Insulin Analog (NovoLog®) at 100 U/mL

This solution is a commercial solution of insulin aspart marketed by the company NOVO NORDISK under the name of NovoLog® in the United States of America and Novorapid in Europe. This product is a rapid insulin analog. The excipients of Novolog® are glycerol (16 mg), phenol (1.50 mg/mL), meta-cresol (1.72 mg/mL), zinc (19.6 μg/mL), disodium phosphate dihydrate (1.25 mg/mL), sodium chloride (0.5 mg/mL), sodium hydroxide and hydrochloric acid for the adjustment of the pH (pH 7.2-7.6), and water.

### Example C3: Solution of Rapid Insulin Analog (Apidra®) at 100 U/mL

This solution is a commercial solution of insulin glulisine marketed by the company SANOFI under the name of Apidra®. This product is a rapid insulin analog. The excipients of Apidra® are meta-cresol (3.15 mg/mL), tromethamine (6 mg/mL), sodium chloride (5 mg/mL), polysorbate 20 (0.01 mg/mL), sodium hydroxide and hydrochloric acid for the adjustment of the pH (pH 7.3), and water.

### Example C4: Solution of Slow-Acting Insulin Analog (Lantus®) at 100 U/mL

This solution is a commercial solution of insulin glargine marketed by the company SANOFI under the name of Lantus®. This product is a slow-acting insulin analog. The excipients in Lantus® are zinc chloride (30 μg/mL), meta-cresol (2.7 mg/mL), glycerol (85%) (20 mg/mL), sodium hydroxide and hydrochloric acid for the adjustment of the pH (pH 4) and water.

### Example C5: Solution of Human Insulin (ActRapid) at 100 IU/mL

This solution is a commercial solution of human insulin from NOVO NORDISK sold under the name of ActRapid®. This product is a human insulin. The excipients of ActRapid® are zinc chloride, glycerol, meta-cresol, sodium hydroxide and hydrochloric acid for the adjustment of the pH (pH 6.9-7.8), and water.

### Example C6: Solution of Human Insulin (Umuline Rapide) at 100 IU/mL

This solution is a commercial solution of human insulin from ELI LILLY sold under the name of Umuline Rapide®. This product is a human insulin. The excipients of Umuline Rapide® are glycerol, meta-cresol, sodium hydroxide and hydrochloric acid for the adjustment of the pH (pH 7.0-7.8), and water.

### Partie CA—Compositions Comprising Insulin Alaraine

Preparation method CA1: Preparation of a diluted composition of co-polyamino acid/insulin glargine 50 U/mL at pH 7.1, according to a method using insulin glargine in liquid form (in solution) and a co-polyamino acid in liquid form (in solution).

To a stock solution of co-polyamino acid at pH 7.1 are added concentrated solutions of m-cresol and glycerol in a manner so as to obtain a solution of co-polyamino acid of concentration C_{co-polyamine acid stock/excipients} (mg/mL). The quantity of excipients added is adjusted in a manner so as to obtain a concentration of m-cresol of 35 mM and of glycerol of 184 mM in the composition of co-polyamino acid/insulin glargine 50 U/mL at pH 7.1.

In a sterile jar, a volume V_{insulin glargine} of a commercial solution of insulin glargine marketed under the name of Lantus® at a concentration of 100 U/mL is added to a volume V_{co-polyamino acid stock/excipients} of a solution of co-polyamino acid at concentration C_{co-polyamino acid stock/excipients} (mg/mL) in a manner so as to obtain a diluted composition of co-polyamino acid C_{diluted co-polyamino acid} (mg/mL)/insulin glargine 50 U/mL at pH 7.1. Turbidity appears. The pH is adjusted to pH 7.1 by addition of concentrated NaOH, and the solution is placed under static conditions in an oven at 40° C. for 2 h until complete solubilization. This visually clear solution is placed at +4° C.

Preparation method CA2: Preparation of a concentrated composition of co-polyamino acid/insulin glargine at pH 7.1 with the aid of a co-polyamino acid, according to a method for concentrating a diluted composition.

A composition of co-polyamino acid/insulin glargine 50 U/mL at pH 7.1 described in Example CA1 is concentrated by ultrafiltration through a 3 kDa membrane made of regenerated cellulose (Amicon® Ultra-15 marketed by the company Millipore). After this ultrafiltration step, the retentate is clear, and the concentration of insulin glargine in the composition is determined by reverse phase chromatography (RP-HPLC). The concentration of insulin glargine in the composition is then adjusted to the desired value by dilution in a solution of excipients m-cresol/glycerol in a manner so as to obtain a final concentration of m-cresol of 35 mM and an osmolarity of 300 mOsm/kg. The pH is measured and adjusted to pH 7.1 by addition of concentrated NaOH and HCl. This solution at pH 7.1, visually clear, has a concentration of insulin glargine C_{insulin glargine} (U/mL) and a concentration of co-polyamino acid C_{co-polyamino acid} (mg/mL)=C_{diluted co-polyamino acid} (mg/mL)×C_{insulin glargine} (U/mL)/50 (U/mL).

According to this preparation method CA2, compositions of co-polyamino acid/insulin glargine were prepared, for example, with concentrations of insulin glargine of 200 U/mL and 400 U/mL at pH 7.1.

### Example CA3: Preparation of Compositions of Co-Polyamino Acid/Insulin Glargine 200 U/mL at pH 7.1

Compositions of co-polyamino acid/insulin glargine 200 U/mL are prepared according to the method described in Example CA2 in a manner so as to obtain a concentration of insulin glargine C_{insulin glargine}=200 U/mL and a concentration of co-polyamino acid C_{co-polyamino acid} (mg/mL).

These compositions are presented in Table 3.

TABLE 3
Compositions of insulin glargine (200
U/mL) in presence of copolyamino acid.

| | | Concentration in | Insulin |
|---|---|---|---|
| | Copolyamino | copolyamino acid | glargine |
| Composition | acid | (in mg/ml) | (U/mL) |
| CA3-1 | B3 | 5 | 200 |
| CA3-2 | B4 | 7 | 200 |
| CA3-4 | B5 | 10 | 200 |
| CA3-5 | B6 | 5 | 200 |
| CA3-6 | B7 | 5 | 200 |
| CA3-8 | B11 | 6 | 200 |
| CA3-9 | B14 | 7 | 200 |

### Partie CB—Compositions Comprising Insulin Glargine and Insulin Lispro

### Preparation Method CB1: Preparation of a Diluted Composition of Co-Polyamino Acid/Insulin Glargine 43 (U/mL)/Insulin Lispro 13.5 (U/mL)

To a volume V_{co-polyamino acid/diluted insulin glargine} of the diluted composition of co-polyamino acid/insulin glargine 50 U/mL at pH 7.1 described in Example CA1 is added a volume V_{insulin lispro} of a commercial solution of insulin lispro Humalog® at 100 U/mL and water in a manner so as to obtain a composition of co-polyamino acid/insulin glargine 43 (U/mL)/insulin lispro 13.5 (U/mL).

### Preparation Method CB2: Preparation of a Concentrated Composition of Co-Polyamino Acid/Insulin Glargine/Insulin Lispro at pH 7.1

A composition of co-polyamino acid/insulin glargine 43 (U/mL)/insulin lispro 13.5 (U/mL) described in Example CB1 is concentrated by ultrafiltration through a 3 kDa membrane made of regenerated cellulose (Amicon® Ultra-15 marketed by the company MILLIPORE). After completion of this ultrafiltration step, the retentate is clear, and the concentration of insulin glargine in the composition is determined by reverse phase chromatography (RP-HPLC). The concentrations of insulin glargine and insulin lispro in the composition are then adjusted to the desired value by dilution in a solution of excipients m-cresol/glycerol in a manner so as to obtain a final concentration of m-cresol of 35 mM and an osmolarity of 300 mOsm/kg. The pH is measured and adjusted if necessary to pH 7.1 by addition of concentrated NaOH and HCl. This solution at pH 7.1, visually clear, has a concentration of insulin glargine C_{insulin glargine} (U/mL), a concentration of insulin lispro C_{insulin lispro}=C_{insulin glargine}×0.33, and a concentration of co-polyamino acid C_{co-acid} (mg/mL)=C_{diluted co-polyamino acid} (mg/mL)×Cᵢₙₛᵤₗᵢₙ (U/mL)/50 (U/mL).

### Example CB3: Preparation of Compositions of Co-Polyamino Acid/Insulin Glargine 200 U/mL/Insulin Lispro 66 U/mL at pH 7,1

Compositions of copolyamino acid/insulin glargine 200 U/mL/insulin lispro 66 U/mL are prepared according to the method described in Example CB2 in a manner so as to obtain a concentration of insulin glargine C_{insulin glargine}=200 U/mL, a concentration of insulin lispro C_{insulin lispro}=66 U/mL and a concentration of copolyamino acid C_{copolyamino acid} (mg/mL).

These compositions are presented in Table 4.

TABLE 4
Compositions of insulin glargine (200 U/mL) and of insulin
lispro (66 U/mL) in presence of copolyamino acid.

| | | Concentration in | Insulin | Insulin |
|---|---|---|---|---|
| | Copolyamino | copolyamino acid | glargine | lispro |
| Composition | acid | (in mg/ml) | U/ml | U/ml |
| CB3-1 | B1 | 17 | 200 | 66 |
| CB3-2 | B4 | 5 | 200 | 66 |
| CB3-3 | B3 | 7 | 200 | 66 |
| CB3-4 | | 8 | 200 | 66 |
| CB3-6 | B5 | 10 | 200 | 66 |
| CB3-7 | B6 | 5 | 200 | 66 |
| CB3-8 | B7 | 5 | 200 | 66 |
| CB3-10 | B11 | 6 | 200 | 66 |
| CB3-11 | B12 | 8 | 200 | 66 |
| CB3-12 | B14 | 7 | 200 | 66 |

The previously obtained compositions (CA3 to CB3) are injectable physically stable compositions.

### Part D—Results

### Physical Stability of the Above Prepared Compositions

### Example D1: Accelerated Stability at 25° C. Under Dynamic Conditions

3 3-mL vials filled with 1 mL of composition copolyamino acid/insulin glargine or copolyamino acid/insulin glargine/prandial insulin are placed vertically in an orbital stirrer. The stirrer is placed in an oven at 25° C., and the vials are subjected to stirring at 50 or 250 rpm. The vials are inspected visually daily/weekly in order to detect the appearance of visible particles or turbidity. This inspection is carried out according to the recommendations of the European Pharmacopoeia (EP 2.9.20): the vials are subjected to illumination of at least 2000 lux and are observed on a white background and on a black background. The number of days of stability corresponds to the duration after which at least 2 vials present visible particles or are turbid.

The results of accelerated stability (obtained with different compositions) are in Table 5 and Table 6.

TABLE 5
results of the stabilities of the compositions of co-
polyamino acid/insulin glargine (200 U/mL) at 25°
C. under dynamic conditions (with stirring at 250 rpm).

| | Copolyamino | concentration | Stability |
|---|---|---|---|
| Composition | acid | (mg/mL) | in days |
| CA3 | — | — | * |
| CA3-1 | B4 | 5 | >24 |
| CA3-2 | B3 | 7 | >24 |
| CA3-5 | B6 | 5 | >24 |
| CA3-6 | B7 | 5 | >24 |
| CA3-8 | B11 | 6 | >24 |

(* A precipitate appears when the pH of the insulin glargine solution is adjusted at pH 7).

TABLE 6
results of the stabilities of the compositions of co-polyamino
acid/insulin glargine (200 U/mL)/insulin lispro (66 U/mL) at
25° C. under dynamic conditions (with stirring at 250 rpm).

| | Copolyamino | Concentration | Stability |
|---|---|---|---|
| Composition | acid | (mg/mL) | in days |
| CB3 | — | — | * |
| CB3-1 | B1 | 17 | >10 |
| CB3-2 | B4 | 5 | >20 |
| CB3-3 | B3 | 7 | >20 |
| CB3-4 | | 8 | >20 |
| CB3-6 | B5 | 10 | >20 |
| CB3-7 | B6 | 5 | >20 |
| CB3-8 | B7 | 5 | >20 |
| CB3--12 | B9 | 6 | >20 |
| CB3-10 | B11 | 6 | >20 |
| CB3-11 | B12 | 8 | >20 |

(* A precipitate appears when the pH of the insulin glargine solution is adjusted at pH 7).

Compositions according to the invention with insulin glargine and with insulin glargine and insulin lispro present a good stability under dynamic conditions.

### Example D2: Accelerated Stability at 30° C. Under Static Conditions

5 3 mL vials filled with 1 mL of a composition are vertically placed in an oven kept at 30° C. The vials are inspected visually daily in order to detect the appearance of visible particles or turbidity. This inspection is carried out according to the recommendations of the European Pharmacopoeia (EP 2.9.20): the vials are subjected to illumination of at least 2000 lux and are observed on a white background and on a black background. The number of weeks of stability corresponds to the duration after which at least half of the vials present visible particles or are turbid.

These results are in agreement with the US pharmacopeia (USP <790>).

The results of accelerated stability (obtained with different compositions) are in Table 8a and 8b hereafter.

TABLE 8a
results of the stabilites of compositions copolyamino acid/insulin
glargine (200 U/mL) at 30° C. under static conditions.

| | | Stability at |
|---|---|---|
| | Copolyamino | 30° C. in static |
| Compositions | acid | (in weeks) |
| CA3 | — | * |
| CA3-2 | B4 | >16 |
| CA3-1 | B3 | >19 |
| CA3-9 | B14 | >19 |
| CA3-4 | B5 | >19 |
| CA3-5 | B6 | >19 |
| CA3-6 | B7 | >18 |
| CA3-8 | B11 | >19 |

(* A precipitate appears when the pH of the insulin glargine solution is adjusted at pH 7).

TABLE 8b
results of the stabilites of compositions copolyamino
acid/insulin glargine (200 U/mL)/insulin lispro
(66 UI/mL) at 30° C. under static conditions.

| | | Stability at |
|---|---|---|
| | Copolyamino | 30° C. in static |
| Composition | acid | (in weeks) |
| CB3 | — | * |
| CB3-1 | B1 | >16 |
| CB3-2 | B4 | >16 |
| CB3-4 | B3 (8) | >16 |
| CB3-12 | B14 | >16 |
| CB3-6 | B5 | >16 |
| CB3-7 | B6 | >16 |
| CB3-8 | B7 | >16 |
| CB3-10 | B11 | >16 |
| CB3-11 | B12 | >16 |

(* A precipitate appears when the pH of the insulin glargine solution is adjusted at pH 7).

Compositions according to the invention with insulin glargine and with insulin glargine and insulin lispro present a good stability under static conditions at 30° C.

### Example D3: Accelerated Stability at 40° C. Under Static Conditions

The accelerated stability at 40° C. under static conditions of compositions was also tested using the same method as described in Example D2.

These results are in agreement with the US pharmacopeia (USP <790>).

The results of accelerated stability (obtained with different compositions) are in Table 8c hereafter.

TABLE 8c
results of the stabilites of compositions copolyamino
acid/insulin glargine (200 U/mL)/insulin lispro
(66 UI/mL) at 40° C. under static conditions.

| | | Stabilite at 40° C. |
|---|---|---|
| | Copolyamino | under static conditions |
| Composition | acid | (in weeks) |
| CB3 | — | * |
| CB3-2 | B3 | >3 |
| CB3-4 | B3 (8) | >3 |

(* A precipitate appears when the pH of the insulin glargine solution is adjusted at pH 7).

Compositions according to the invention with insulin glargine and insulin lispro present a good stability under static conditions at 40° C.

### Example D3: Precipitation of Insulin Glargine in Compositions Copolyamino Acid/Insulin Glargine at 200 U/mL

1 mL of copolyamino acid/insulin glargine solution prepared in Example CA3 is added in a 2 mL of a PBS solution (Phosphate Buffer Saline, buffer phosphate saline) containing 20 mg/mL of BSA ((Bovine serum Albumine, serum albumine bovine)). The mixture PBS/BSA simulates the composition in the subcutaneous medium. A precipitate appears.

A centrifugation at 4000 rpm is carried out in order to separate the precipitate from the supernatant. Next, the insulin glargine is assayed in the supernatant by RP-HPLC. The result is that insulin glargine is present in majority proportion in a precipitated form.

### Results are in Table 7.

TABLE 7
Compositions copolyamino acid/insulin glargine (200 U/mL);
solubilization/precipitation of insulin glargine.

| | | | Concentra- | | |
|---|---|---|---|---|---|
| | | | tion in | Solubiliza- | Precipita- |
| | Copoly- | | copoly- | tion of | tion of |
| Composi- | amino | Insulin | amino acid | insulin | insulin |
| tion | acid | glargine | (in mg/ml) | glargine | glargine |
| | — | 200 | — | NO | NA |
| CA3-1 | B4 | 200 | 5 | YES | YES |
| CA3-2 | B3 | 200 | 7 | YES | YES |
| CA3-4 | B5 | 200 | 10 | YES | YES |
| CA3-5 | B6 | 200 | 5 | YES | YES |
| CA3-9 | B14 | 200 | 7 | YES | YES |

Copolyamino acids allow for the preparation of a solution of insulin glargine at neutral pH and for the precipitation of the latter when said solution is added in a medium simulating the subcutaneous medium.

### Example D4: Precipitation of Insulin Glargine in Compositions Copolyamino Acid/Insulin Glargine 200 U/mL/Insulin Lispro 66 U/mL at pH 7.1

1 mL of solution of co-polyamino acid/insulin glargine/insulin lispro prepared in Example CB3 is added to 2 mL of a PBS solution containing 20 mg/mL of BSA (bovine serum albumin). The PBS/BSA mixture simulates the composition of the subcutaneous environment. A precipitate appears.

A centrifugation at 4000 rpm is carried out in order to separate the precipitate from the supernatant. Next, the insulin glargine is assayed in the supernatant by RP-HPLC. The result is that insulin glargine is found in majority proportion in a precipitated form. The results are presented in Table 8.

TABLE 8
Compositions copolyamino acid/insulin glargine (200 U/mL)/insulin lispro
(66 U/mL); solubilization and precipitation of insulin glargine.

| | | | | Concentration en | Solubilization | Precipitation |
|---|---|---|---|---|---|---|
| | Copolyamino | Insulin | | copolyamino acid | of insulin | of insulin |
| Compositions | acid | glargine | Lispro | (in mg/ml) | glargine | glargine |
| CB3-1 | B1 | 200 | 66 | 17 | YES | YES |
| CB3-2 | B4 | 200 | 66 | 5 | YES | YES |
| CB3-3 | B3 | 200 | 66 | 7 | YES | YES |
| CB3-6 | B5 | 200 | 66 | 10 | YES | YES |
| CB3-7 | B6 | 200 | 66 | 5 | YES | YES |
| CB3-12 | BB14 | 200 | 66 | 7 | YES | YES |
| CB3-8 | B7 | 200 | 66 | 5 | YES | YES |
| CB3-10 | B11 | 200 | 66 | 6 | YES | YES |
| CB3-11 | B12 | 200 | 66 | 8 | YES | YES |
| CB3-9 | B13 | 200 | 66 | 5 | YES | YES |

Copolyamino acids allow for the preparation of a solution of insulin glargine in presence of insulin lispro at neutral pH and for the precipitation of the latter when said solution is added in a medium simulating the subcutaneous medium.

### Example D5: Pharmacodynamy in Dogs

Studies in dogs were carried out for the purpose of evaluating the pharmacodynamics of insulin after administration of the composition of copolyamino acid B11 and insulins (composition CB3-10).

The hypoglycemic effects of this composition were compared to those of simultaneous but separate injections of insulin glargine (Lantus®) (pH 4) and a prandial insulin lispro (Humalog®) in the proportions of 75% of insulin glargine/25% of insulin lispro (dose/dose).

Ten animals that had fasted for approximately 18 hours received injections in the neck above the interscapular region at the dose of 0.67 U/kg. In the hour preceding the insulin injection 3 blood samples were drawn in order to determine the basal level of glucose. The glycemia is determined over 24 h by means of a glucometer.

The mean pharmacokinetic curves of glucose expressed in deviation percentage of the basal level are represented in FIG. 1.

The pharmacodynamic results obtained with the separate and simultaneous administrations of Humalog® (example C1) and Lantus® (example C4) in comparison to those obtained with the composition described in Example CB3-10 are presented in FIG. 1. The hypoglycemic activity of the composition described in Example CB3-10 is biphasic. The first rapid phase is defined by a pronounced decrease of glycemia for approximately 60 minutes, which is characteristic of the rapid effect of insulin lispro. This first phase is also visible with the double Lantus®/Humalog® injection, indicating that the composition according to the invention does not modify the rapid character of Humalog®. After approximately 60 minutes, the glycemia rises up to 3 hours, before a second slower phase characterized by a less pronounced hypoglycemic activity lasting up to 18-20 hours post injection. This basal second phase is characteristic of the basal effect of insulin glargine, also visible with the double injection, indicating that the effect is indeed maintained with the composition according to the invention described in Example CB3-10.

1-32. (canceled)

33. A physically stable composition in the form of an injectable aqueous solution, which pH is comprised from 6.0 to 8.0, comprising at least-:
a) a basal insulin which isoelectric point (pI) is comprised from 5.8 and 8.5 and
b) a co-polyamino acid according to formula I
Q[Hy]ⱼ[PLG]ₖ Formula I
wherein—:
j≥1; k≥2
the co-polyamino acid according to formula I bearing carboxylate charges and consisting of at least two chains of PLG glutamic or aspartic units bound together with a linear or branched radical or spacer Q[-*]ᵢ (i≥3 with i=j+k) at least trivalent consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl groups the radical Q[-*]ᵢ bearing at least a monovalent hydrophobic radical -Hy according to formula X;
the radical or spacer Q[-*]ᵢ being bound to at least two chains of PLG glutamic or aspartic units by an amide function and,
the radical or spacer Q[-*]ᵢ being bound to at least a hydrophobic radical -Hy according to formula X by an amide function.

34. The composition according to claim 33, wherein the radical or spacer Q[-*]ᵢ (i≥3) is represented by a radical according to formula II:
Q[-**]ᵢ=([Q′]_{q})[-*]ᵢ Formula II
wherein 1≤q≤5
radicals Q′ being identical or different and chosen in the group consisting of radicals according to the following formula III to VI, to form Q[-*]ᵢ (i≥3) 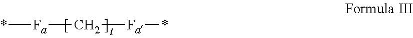
wherein 1≤t≤8 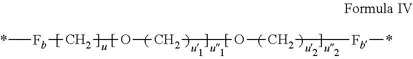
wherein:
at least one of u₁″ or u₂″ is different from 0,
if u₁″≠0 then u₁′≠0 and if u₂″≠0 then u₂′≠0,
u₁′ and u₂′ are identical or different and,
2≤u≤4,
0≤u₁′≤4,
0≤u₁″≤4,
0≤u₂′≤4
0≤u₂″≤4 and, 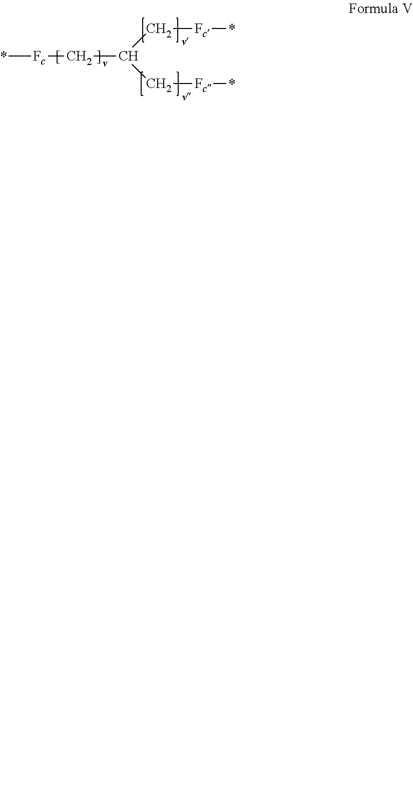
wherein:
v, v′ and v″ identical or different,
v+v′+v″≤15 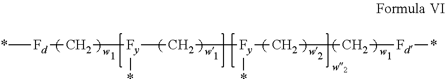
wherein:
w₁′ is different from 0,
0≤w₂″≤1,
w₁≤6 and w₁′≤6 and/or w₂≤6 and w₂′≤6,
with Fx=Fa, Fb, Fc, Fd, Fa′, Fb′, Fc′, Fc″ and Fd′ identical or different represents functions —NH— or —CO— and Fy represents a trivalent nitrogen atom —N═,
two radicals Q′ being bound between them by a covalent bond between a carboxyl function, Fx=—CO—, and an amine function Fx=—NH— or Fy=—N═, thus forming an amide bond,

35. The composition according to claim 33, wherein the hydrophobic radical -Hy is chosen among radicals according to formula X as defined hereafter: 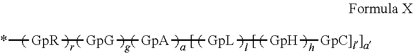
wherein:
GpR is chosen among radicals according to formula VII, VII′ or VII″—: 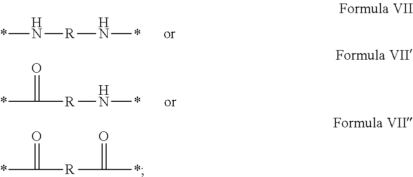
GpG and GpH identical or different are chosen among radicals according to formula XI or XI′: 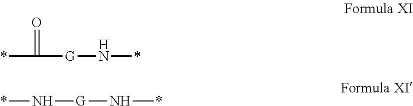
GpA is chosen among radicals according to formula VIII 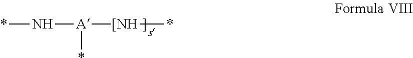
wherein A′ is chosen among radicals according to formula VIII′, VIII″ or VIII′″ 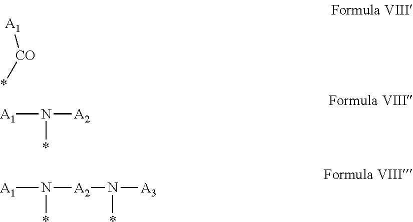
GpL is chosen among radicals according to formula XII 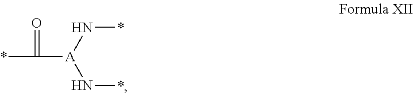
GpC is a radical according to formula IX: 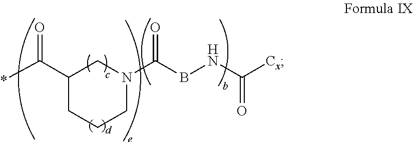
the * indicate the binding sites of the different groups bound by amide functions; a is an integer equal to 0 or to 1 and a′=1 if a=0 and a′=1, 2 or 3 if a=1;
a′ is an integer equal to 1, to 2 or to 3
b is an integer equal to 0 or to 1;
c is an integer equal to 0 or to 1, and if c is equal to 0 then d is equal to 1 or to 2;
d is an integer equal to 0, to 1 or to 2;
e is an integer equal to 0 or to 1;
g is an integer equal to 0, to 1, to 2, to 3 to 4 to 5 or to 6;
h is an integer equal to 0, to 1, to 2, to 3 to 4 to 5 or to 6;
l is an integer equal to 0 or 1 and l′=1 if l=0 and l′=2 if l=1;
r is an integer equal to 0 or to 1, and
s′ is an integer equal to 0 or 1;
A, A₁, A₂ and A₃ identical or different are linear or branched alkyl radicals comprising from 1 to 6 carbon atoms;
B is a linear or branched alkyl radical, optionally comprising an aromatic ring, comprising from 1 to 9 carbon atoms;
Cₓ is a monovalent linear or branched alkyl radical, in which x indicates the number of carbon atoms and:
when the hydrophobic radical -Hy carries 1 -GpC, then 9≤x≤25,
when the hydrophobic radical -Hy carries 2 -GpC, then 9≤x≤15,
when the hydrophobic radical -Hy carries 3 -GpC, then 7≤x≤13,
when the hydrophobic radical -Hy carries 4 -GpC, then 7≤x≤11,
when the hydrophobic radical -Hy carries at least 5 -GpC then, 6≤x≤11,
G is a branched alkyl radical from 1 to 8 carbon atoms the alkyl radical bearing one or several free carboxylic acid function(s),
H is a branched alkyl radical from 1 to 8 carbon atoms the alkyl radical bearing one or several free carboxylic acid function(s),
R is a radical chosen in the group consisting of a divalent linear or branched alkyl radical, comprising from 1 to 12 carbon atoms, a divalent linear or branched alkyl radical, comprising from 1 to 12 carbon atoms bearing one or several functions —CONH₂ or a non-substituted ether or polyether radical comprising from 4 to 14 carbon atoms and from 1 to 5 oxygen atoms:
Hydrophobic radical(s) -Hy according to formula X being bound to Q:
via a covalent bond between a carbonyl of the hydrophobic radical -Hy and nitrogen atom carried by Q thus forming an amide function from the reaction of an amine function carried by the precursor of Q and an acid function carried by the precursor -Hy′ of the hydrophobic radical -Hy, and
via a covalent bond between a nitrogen atom of the hydrophobic radical -Hy and a carbonyl carried by Q, thus forming an amide function from the reaction of an amine function of the precursor -Hy′ of the hydrophobic radical -Hy and an acid function carried by the precursor of the radical Q,
the ratio M between the number of hydrophobic radicals and the number of glutamic or aspartic units being comprised from 0<M≤0.5;
when several hydrophobic radicals are carried by a co-polyamino acid then they are identical or different,
the degree of polymerization DP of glutamic or aspartic units for PLG chains is between 5 and 250;
the free carboxylic acid functions being in the form of alkaline cation salt chosen in the group consisting of Na⁺ and K⁺.

36. The composition according to claim 33, wherein the co-polyamino acid bearing carboxylate charges and at least a hydrophobic radical -Hy is chosen among co-polyamino acids according to the following formula XXXa: 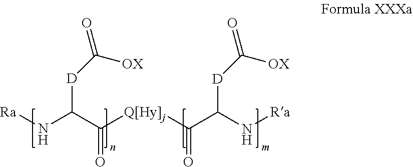
wherein,
D represents, independently, either a —CH₂— group (aspartic unit) or a —CH₂—CH₂— group (glutamic unit),
X represents a cationic entity chosen in the group comprising alkali cations,
Rₐ and Rₐ′, identical or different, are a radical chosen in the group consisting of a H, a C2 to C10 linear acyl group, a C3 to C10 branched acyl group, a benzyl, a terminal «amino acid» unit and a pyroglutamate,
Q, Hy and j as defined in claim 33,
n+m represents the degree of polymerisation DP of the copolyamino acid, namely the mean number of monomeric units per co-polyamino acid chain and 5≤n+m≤250.

37. The composition according to claim 33, wherein the co-polyamino acid bearing carboxylate charges and at least a hydrophobic radical -Hy is chosen among co-polyamino acids according to the following formula XXXa′: 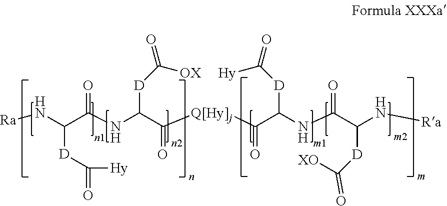
D represents, independently, either a —CH₂— group (aspartic unit) or a —CH₂—CH₂— group (glutamic unit),
X represents a cationic entity chosen in the group comprising alkali cations,
Q, Hy and j as defined in claim 33,
Rₐ and Rₐ′, identical or different, are a radical chosen in the group consisting of a H, a C2 to C10 linear acyl group, a C3 to C10 branched acyl group, a benzyl, a terminal «amino acid» unit and a pyroglutamate,
n₁+m₁ represents the number of glutamic or aspartic units of PLG chains co-polyamino acid bearing a radical -Hy,
n₂+m₂ represents the number of glutamic or aspartic units of PLG chains of the co-polyamino acid not bearing a radical -Hy,
n₁+n₂=n and m₁+m₂=m,
n+m represents the degree of polymerisation DP of the co-polyamino acid, namely mean number of monomeric units per co-polyamino acid chain and 5≤n+m≤250.

38. The composition according to claim 33, wherein the co-polyamino acid bearing carboxylate charges and at least a hydrophobic radical -Hy is chosen among co-polyamino acids according to the following formula XXXa″: 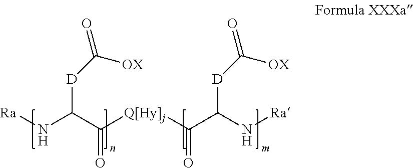
D represents, independently, either a —CH₂— group (aspartic unit) or a —CH₂—CH₂— group (glutamic unit),
X represents a cationic entity chosen in the group comprising alkali cations,
Q, Hy and j as defined in claim 33,
Rₐ and Rₐ′, identical or different, are at least a hydrophobic radical -Hy and a radical chosen in the group consisting of -Hy, a H, a C2 to C10 linear acyl group, a C3 to C10 branched acyl group, a benzyl, a terminal «amino acid» unit and a pyroglutamate,
n+m represents the degree of polymerisation DP of the co-polyaminoacid, namely the mean number of monomeric units per co-polyamino acid chain and 5≤n+m≤250.

39. The composition according to claim 33, wherein the co-polyamino acid bearing carboxylate charges and at least a hydrophobic radical -Hy is chosen among co-polyamino acids according to the following formula XXXb: 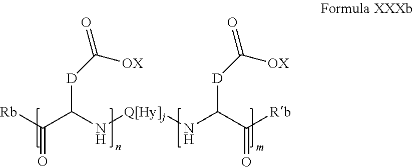
wherein,
D represents, independently, either a —CH₂— group (aspartic unit) or a —CH₂—CH₂— group (glutamic unit),
X represents a cationic entity chosen in the group comprising alkali cations,
R_{b} and R_{b}′, identical or different, are a —NR′R″ radical, R′ and R″ identical or different being chosen in the group consisting of H, C2 to C10 linear or branched or cyclic alkyls, the benzyl and the R′ and R″ alkyls may form together one or several saturated, unsaturated and/or aromatic carbon rings and/or may comprise heteroatoms, chosen in the group consisting of 0, N and S;
Q, Hy and j as defined in claim 33,
n+m represents the degree of polymerisation DP of the co-polyamino acid, namely the mean number of monomeric units per co-polyamino acid chain and 5≤n+m≤250.

40. The composition according to claim 33, wherein the co-polyamino acid bearing carboxylate charges and at least a hydrophobic radical -Hy is chosen among co-polyamino acids according to the following formula XXXb′: 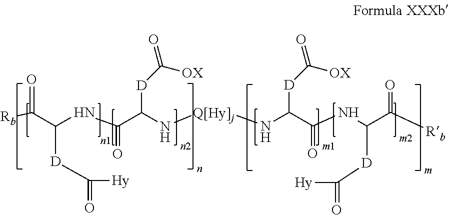
D represents, independently, either a —CH₂— group (aspartic unit) or a —CH₂—CH₂— group (glutamic unit),
X represents a cationic entity chosen in the group comprising alkali cations,
Q, Hy and j as defined in claim 33,
R_{b} and R_{b}′, identical or different, are a —NR′R″ radical, R′ and R″ identical or different being chosen in the group consisting of H, C2 to C10 linear or branched or cyclic alkyls, the benzyl and the R′ and R″ alkyl may form together one or several saturated, unsaturated and/or aromatic carbon rings and/or may comprise heteroatoms, chosen in the group consisting of 0, N and S;
n1+m1 represents the number of glutamic or aspartic units of PLG chains of the co-polyamino acid bearing a radical -Hy,
n2+m2 represents the number of glutamic or aspartic units of PLG chains of the co-polyamino acid not bearing a radical -Hy,
n1+n2=n and m1+m2=m,
n+m represents the degree of polymerisation DP of co-polyamino acid, namely the mean number of monomeric units in a co-polyamino acid chain and 5≤n+m≤250.

41. The composition according to claim 33, wherein the co-polyamino acid bearing carboxylate charges and at least a hydrophobic radical -Hy is chosen among co-polyamino acids according to the following formula XXXb″: 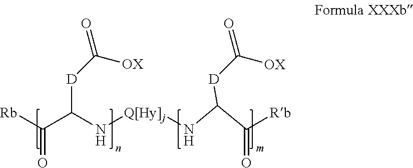
D represents, independently, either a —CH₂— group (aspartic unit) or a —CH₂—CH₂— group (glutamic unit),
X represents a cationic entity chosen in the group comprising alkali cations,
R_{b} and R_{b}′, identical or different, are at least a hydrophobic radical -Hy and a radical chosen in the group consisting of a hydrophobic radical -Hy and a —NR′R″ radical, R′ and R″ identical or different being chosen in the group consisting of H, C2 to C10 linear or branched or cyclic alkyls, the benzyl and the R′ and R″ alkyl may form together one or several saturated, unsaturated and/or aromatic carbon rings and/or may comprise heteroatoms, chosen in the group consisting of O, N and S;
Q, Hy and j as defined in claim 33,
n+m represents the degree of polymerisation DP of co-polyamino acid, namely the mean number of monomeric units per co-polyamino acid chain and 5≤n+m≤250.

42. The composition according to claim 33, wherein the basal insulin which isoelectric point is comprised from 5.8 to 8.5 is insulin glargine.

43. The composition according to claim 33, wherein it comprises between 40 and 500 U/mL of basal insulin which isoelectric point is comprised from 5.8 to 8.5.

44. The composition according to claim 33, wherein the concentration of co-polyamino acid bearing carboxylate charges and hydrophobic radicals is at most of 60 mg/mL.

45. The composition according to claim 33, wherein the concentration of co-polyamino acid bearing carboxylate charges and hydrophobic radicals is at most of 40 mg/mL.

46. The composition according to claim 33, wherein the concentration of co-polyamino acid bearing carboxylate charges and hydrophobic radicals is at most of 20 mg/mL.

47. The composition according to claim 33, wherein the concentration of co-polyamino acid bearing carboxylate charges and hydrophobic radicals is at most of 10 mg/mL.

48. The composition according to claim 33, wherein it further comprises a prandial insulin.

49. The composition according to claim 48, wherein the prandial insulin is human insulin.

50. The composition according to claim 48, wherein in total it comprises between 40 and 500 U/mL of insulin with a combination of prandial insulin and basal insulin which isoelectric point is comprised from 5.8 to 8.5.

51. The composition according to claim 48, wherein the proportions between the basal insulin which isoelectric point is comprised from 5.8 to 8.5 and the prandial insulin are in percentage from 25/75, 30/70, 40/60, 50/50, 60/40, 70/30, 80/20 or 90/10.

52. The composition according to claim 33, wherein it further comprises a gastrointestinal hormone.

53. The composition according to claim 52, wherein the gastrointestinal hormone is chosen in the group consisting of exenatide, liraglutide, lixisenatide, albiglutide and dulaglutide, their analogues or derivatives and their pharmaceutically acceptable salts.

54. The composition according to claim 52, wherein the gastrointestinal hormone is the dulaglutide their analogues or derivatives and their pharmaceutically acceptable salts.

55. The composition according to claim 52, wherein the gastrointestinal hormone is exenatide their analogues or derivatives and their pharmaceutically acceptable salts.

56. The composition according to claim 52, wherein the gastrointestinal hormone is liraglutide their analogues or derivatives and their pharmaceutically acceptable salts.

57. The composition according to claim 52, wherein the gastrointestinal hormone is lixisenatide their analogues or derivatives and their pharmaceutically acceptable salts.

58. The composition according to claim 52, wherein the concentration of gastrointestinal hormone is comprised within a range from 0.01 to 10 mg/mL.

59. The composition according to claim 53, wherein it comprises between 40 U/mL and 500 U/mL of basal insulin which isoelectric point is comprised from 5.8 to 8.5 and from 0.05 to 0.5 mg/mL of exenatide.

60. The composition according to claim 53, wherein it comprises between 40 U/mL and 500 U/mL of basal insulin which isoelectric point is comprised from 5.8 to 8.5 and from 1 to 10 mg/mL of liraglutide.

61. The composition according to claim 53, wherein it comprises between 40 U/mL and 500 U/mL of basal insulin which isoelectric point is comprised from 5.8 to 8.5 and de 0.01 to 1 mg/mL of lixisenatide.

62. A single-dose formulation comprising a composition according to claim 33.

63. A co-polyamino acid according to formula I
Q[Hy]ⱼ[PLG]ₖ Formula I
wherein:
j≥1; k≥2
the co-polyamino acid according to formula I bearing carboxylate charges and consisting of at least two chains of PLG glutamic or aspartic units bound together with a linear or branched radical or spacer Q[-]ᵢ (i≥3 with i=j+k) at least trivalent consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl functions the radical Q[-*]ᵢ bearing at least a monovalent hydrophobic radical -Hy;
the radical or spacer Q[-*]ᵢ being linked to at least two chains of PLG glutamic or aspartic units by an amide function and,
the radical or spacer Q[-*]ᵢ being linked to at least a hydrophobic radical -Hy according to formula X below defined by an amide function,
the amide functions binding the radical or spacer Q[-*]ᵢ to at least two chains of glutamic or aspartic units resulting from a reaction between an amine function and an acid function respectively carried either by the precursor Q′ of the radical or spacer Q[-*]ᵢ or by a glutamic or aspartic unit,
the amide function binding the radical or spacer Q[-*]ᵢ to at least a hydrophobic radical -Hy according to formula X resulting form the reaction between an amine function and an acid function respectively carried either by the precursor Q′ of the radical or spacer Q[-*]ᵢ or by the precursor Hy′ of the hydrophobic radical -Hy,
radical -Hy being defined in claim 35,
radical or spacer Q[-*]ᵢ (i≥3) by the radical according to formula II:
Q[-*]ᵢ=([Q′]_{q})[-*]ᵢ Formula II
wherein 1≤q≤5
radicals Q′ being identical or different and chosen in the group consisting of radicals according to the following formula III to VI, to form Q[-*]ᵢ (i≥3) 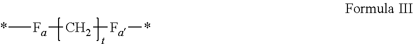
wherein 1≤t≤8 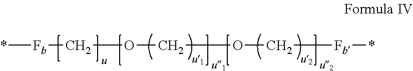
wherein:
at least one of u₁″ or u₂″ is different from 0,
if u₁″≠0 then u₁′≠0 and if u₂″≠0 then u₂′≠0,
u₁′ and u₂′ are identical or different and,
2≤u≤4,
0≤uᵢ′≤4,
0≤u₁″≤4,
0≤u₂′≤4,
0≤u₂″≤4 and, 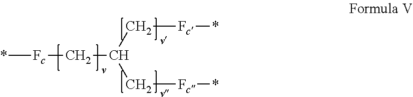
wherein:
v, v′ and v″ identical or different,
v+v′+v″≤15 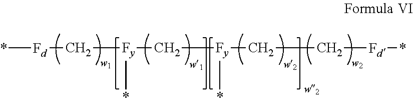
wherein:
w₁′ is different from 0,
0≤w₂″≤1,
w₁≤6 and w₁′≤6 and/or w₂≤6 and w₂′≤6,
with Fx=Fa, Fb, Fc, Fd, Fa′, Fb′, Fc′, Fc″ and Fd′ identical or different represents functions —NH— or —CO— and Fy represents the trivalent nitrogen atom —N═,
two radicals Q′ being bound between them by a covalent bond between the carboxyl function, Fx=—CO—, and the amine function Fx=—NH— or Fy=—N═, thus forming the amide bond.

64. A co-polyamino acid according to formula Ia precursor of co-polyamino acid according to formula I as defined in claim 63
Q″[PLG]ₖ Formula Ia
wherein:
k≥2
the co-polyamino acid according to formula Ia bearing carboxylate charges and consisting of at least two chains of PLG glutamic or aspartic units bound together with a linear or branched radical or spacer Q″[-*]ₖ consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl functions,
the radical or spacer Q″[-*]k being bound to at least two chains of PLG glutamic or aspartic units by an amide function and, bearing after binding to the at least two chains of PLG glutamic or aspartic units at least j amine or acid reactives functions, free,
the amide functions binding the radical or spacer Q″[-*]ₖ to at least two chains of glutamic or aspartic units resulting from a reaction between an amine function and an acid function respectively carried either by the precursor Q′ of the radical or spacer Q″[-]ₖ or by glutamic or aspartic unit,
the radical or spacer Q″[-*]k being chosen among radicals according to formula
Q″[-*]ₖ=([Q′]_{q}[-*]ₖ, wherein 1≤q≤5
radicals Q′ being identical or different and chosen in the group consisting of radicals according to formula III to VI defined above, to form Q[-*]ₖ (k≥3), where functions Fx=Fa, Fb, Fc, Fd, Fa′, Fb′, Fc′, Fc″ et Fd′ identical or different representing —NH— or —CO— functions and Fy representing a trivalent nitrogen atom —N═,
two radicals Q′ being linked together with a covalent bound between a carbonyl function, Fx=—CO—, and an amine function Fx=—NH— or Fy=—N═, forming thereby an amide bond,
and when the at least reactive function is not linked to a radical Q′ or to at least two chains of glutamic or aspartic units they constitute carboxylic acid or amine free functions.

65. A precursor of co-polyamino acid according to formula I as defined in claim 63, the precursor being according formula Ib:
Q′″[HY]ⱼ Formula Ib
wherein:
j≥1
the compound according to formula Ib consisting of precursor of radical or spacer Q′″[-*]ⱼ linear or branched consisting of an alkyl chain comprising one or several heteroatoms chosen in the group consisting of nitrogen and oxygen atoms and/or bearing one or several heteroatoms consisting of nitrogen and oxygen atoms and/or radicals bearing one or several heteroatoms consisting of nitrogen and oxygen atoms and/or carboxyl functions, the radical Q′″[-*]ⱼ bearing at least a monovalent hydrophobic radical -Hy bound by amides bonds, and
precursor of radical or spacer Q′″[-*]ⱼ bearing units at least k amine or acid reactives functions, free,
the amides functions binding the radical or spacer Q′″[-*]ⱼ to at least hydrophobic radical -Hy resulting from a reaction between an amine function and an acid function respectively carried either by the precursor Q′ of the radical or spacer Q′″[-*]ⱼ or by the precursor Hy′ of the hydrophobic radical -Hy,
the radical or spacer Q′″[-*]ⱼ being chosen among radicals according formula Q[-*]ⱼ=([Q′]_{q}[-*]ⱼ, wherein 1≤q≤5
radicals Q′ being identical or different and chosen in the group consisting of radicals according to formula III to VI defined above, to form Q[-*]ⱼ (j≥3), where functions Fx=Fa, Fb, Fc, Fd, Fa′, Fb′, Fc′, Fc″ et Fd′ identical or different representing —NH— or —CO— functions and Fy representing a trivalent nitrogen atom —N═,
two radicals Q′ being linked together with a covalent bond and a carbonyl function, Fx=—CO—, and an amine function Fx=—NH— or Fy=—N═, forming thereby an amide bond,
and when the at least two reactive functions are not linked to a radical Q′ or to at least a hydrophobic radical -Hy they constitute carboxylic acid or amine free functions.
